(19) United States
(12) Patent Application Publication
(10) Pub. No.: US 2010/0137449 A1
(43) Pub. Date: Jun. 3, 2010

(54) SUBSTITUTED 1,3-CYCLOPENTADIONE MULTI-TARGET PROTEIN KINASE MODULATORS OF CANCER, ANGIOGENESIS AND THE INFLAMMATORY PATHWAYS ASSOCIATED THEREWITH

(75) Inventors: Matthew L. Tripp, Gig Harbor, WA (US); John G. Babish, Brooktondale, NY (US); Jeffrey S. Bland, Fox Island, WA (US); Veera Konda, Gig Harbor, WA (US); Anu Desai, Gig Harbor, WA (US); Gary Darland, Gig Harbor, WA (US); Brian J. Carroll, Gig Harbor, WA (US); James S. Traub, Boulder, CO (US); Linda M. Pacioretty, Brooktondale, NY (US); Dennis Emma, Gig Harbor, WA (US)

Correspondence Address: McDermott Will & Emery, 600 13th Street, NW, Washington, DC, 20005-3096, US

(73) Assignee: METAPROTEOMICS, LLC, San Clemente, CA (US)

(21) Appl. No.: 12/331,887

(22) Filed: Dec. 10, 2008

Related U.S. Application Data
(60) Provisional application No. 61/012,506, filed on Dec. 10, 2007.

(51) Int. Cl. A61K 31/122; A61P 35/00; A61P 9/10

(52) U.S. Cl. 514/685; 514/689; 514/690

(57) ABSTRACT

Compounds and methods for multi-targeted protein kinase modulation for angiogenesis, cancer treatment or the inflammatory pathways associated with those conditions are disclosed. The compounds and methods disclosed are based on substituted 1,3-cyclopentadione compounds.

### CROSS-REFERENCE TO RELATED APPLICATIONS

This patent application claims priority to U.S. provisional application Ser. No. 60/012,506, filed on Dec. 10, 2007, The contents of the priority application are incorporated herein by reference in their entirety as though fully set forth herein.

### BACKGROUND OF THE INVENTION

1. Field of the Invention

The present invention relates generally to methods and compositions that can be used to treat or inhibit cancers, angiogenesis, and modulate their associated inflammatory pathways susceptible to protein kinase modulation. More specifically, the invention relates to methods and compositions that utilize substituted 1,3-cyclopentadione compounds.

2. Description of the Related Art

Signal transduction provides an overarching regulatory mechanism important to maintaining normal homeostasis or, if dysregulated, acting as a causative or contributing mechanism associated with numerous disease pathologies and conditions. At the cellular level, signal transduction refers to the movement of a signal or signaling moiety within the cell or from outside of the cell to the cell interior. The signal, upon reaching its receptor target, may initiate ligand-receptor interactions requisite to many cellular events, some of which may further act as a subsequent signal. Such interactions serve not only as a series cascade, but also are part of an intricate interacting network or web of signal events capable of providing fine-tuned control of homeostatic processes. This network however can become dysregulated, thereby resulting in an alteration in cellular activity and changes in the program of genes expressed within the responding cell. See, for example, FIG. 1, which displays a simplified version of the interacting kinases regulating regulating the NF-κB signal transduction pathway.

Signal transducing receptors are generally divided into three classes. The first class of receptors are receptors that penetrate the plasma membrane and have some intrinsic enzymatic activity. Representative receptors that have intrinsic enzymatic activities include those that are tyrosine kinases (e.g. PDGF, insulin, EGF and FGF receptors), tyrosine phosphatases (e.g. CD45 [cluster determinant-45] protein of T cells and macrophages), guanylate cyclases (e.g. natriuretic peptide receptors) and serine/threonine kinases (e.g. activin and TGF-β receptors). Receptors with intrinsic tyrosine kinase activity are capable of autophosphorylation as well as phosphorylation of other substrates.

Receptors of the second class are those that are coupled, inside the cell, to GTP-binding and hydrolyzing proteins (termed G-proteins), Receptors of this class that interact with G-proteins have a structure that is characterized by 7 transmembrane spanning domains. These receptors are termed serpentine receptors. Examples of this class are the adrenergic receptors, odorant receptors, and certain hormone receptors (e.g. glucagon, angiotensin, vasopressin and bradykinin).

The third class of receptors may be described as receptors that are found intracellularly and, upon ligand binding, migrate to the nucleus where the ligand-receptor complex directly affects gene transcription,

The proteins that function as receptor tyrosine kinases (RTK) contain four major domains, those being: a) a transmembrane domain, b) an extracellular ligand binding domain, c) an intracellular regulatory domain, and d) an intracellular tyrosine kinase domain. The amino acid sequences of RTKs are highly conserved with those of cAMP-dependent protein kinase (within the ATP and substrate binding regions). RTK proteins are classified into families based upon structural features in their extracellular portions, which include the cysteine rich domains, immunoglobulin-like domains, cadherin domains, leucine-rich domains, Kringle domains, acidic domains, fibronectin type III repeats, discoidin I-like domains, and EGF-like domains. Based upon the presence of these various extracellular domains the RTKs have been sub-divided into at least 14 different families,

Many receptors that have intrinsic tyrosine kinase activity upon phosphorylation interact with other proteins of the signaling cascade. These other proteins contain a domain of amino acid sequences that are homologous to a domain first identified in the c-Src proto-oncogene. These domains are termed SH2 domains.

The interactions of SH2 domain containing proteins with RTKs or receptor associated tyrosine kinases leads to tyrosine phosphorylation of the SH2 containing proteins. The resultant phosphorylation produces an alteration (either positively or negatively) in that activity. Several SH2 containing proteins that have intrinsic enzymatic activity include phospholipase C-γ (PLC-γ), the proto-oncogene c-Ras associated GTPase activating protein (rasGAP), phosphatidylinositol-3-kinase (PI3K), protein tyrosine phosphatase-1C (PTP1C), as well as members of the Src family of protein tyrosine kinases (PTKs).

Non-receptor protein tyrosine kinases (PTK) by and large couple to cellular receptors that lack enzymatic activity themselves. An example of receptor-signaling through protein interaction involves the insulin receptor (IR). This receptor has intrinsic tyrosine kinase activity but does not directly interact, following autophosphorylation, with enzymatically active proteins containing SH2 domains (e.g. PI3K or PLC-γ). Instead, the principal IR substrate is a protein termed IRS-1.

The receptors for the TGF-β superfamily represent the prototypical receptor serine/threonine kinase (RSTK). Multifunctional proteins of the TGF-β superfamily include the activins, inhibins and the bone morphogenetic proteins (BMPs). These proteins can induce and/or inhibit cellular proliferation or differentiation and regulate migration and adhesion of various cell types. One major effect of TGF-β is a regulation of progression through the cell cycle. Additionally, one nuclear protein involved in the responses of cells to TGF-β is c-Myc, which directly affects the expression of genes harboring Myc-binding elements. PKA, PKC, and MAP kinases represent three major classes of non-receptor serine/threonine kinases.

The relationship between kinase activity and disease states is currently being investigated in many laboratories. Such relationships may be either causative of the disease itself or intimately related to the expression and progression of disease associated symptomology. Rheumatoid arthritis, an autoimmune disease, provides one example where the relationship between kinases and the disease are currently being investigated.

Rheumatoid arthritis (RA) is the most prevalent and best studied of the autoimmune diseases and afflicts about 1% of the population worldwide, and for unknown reasons, like other autoimmune diseases, is increasing. RA is characterized by chronic synovial inflammation resulting in progressive bone and cartilage destruction of the joints. Cytokines, chemokines, and prostaglandins are key mediators of inflammation and can be found in abundance both in the joint and blood of patients with active disease. For example, PGE₂ is abundantly present in the synovial fluid of RA patients. Increased PGE₂ levels are mediated by the induction of cyclooxygenase-2 (COX-2) and inducible nitric oxide synthase (iNOS) at inflamed sites. [See, for example van der Kraan P M and van den Berg W B. Anabolic and destructive mediators in osteoarthritis. Curr Opin Clin Nutr Metab Care,3:205-211, 2000; Choy E H S and Panayi G S Cytokine pathways and joint inflammation in rheumatoid arthritis. N Eng J Med, 344:907-916, 2001; and Wong B R, et al. Targeting Syk as a treatment for allergic and autoimmune disorders. Expert Opin Investig Drugs 13:743-762, 2004]

The etiology and pathogenesis of RA in humans is still poorly understood, but is viewed to progress in three phases. The initiation phase occurs where dendritic cells present self antigens to autoreactive T cells. The T cells activate autoreactive B cells via cytokines resulting in the production of autoantibodies, which in turn form immune complexes in joints. In the effector phase, the immune complexes bind Fcf receptors on macrophages and mast cells, resulting in release of cytokines and chemokines causing inflammation and pain. In the final phase, cytokines and chemokines activate and recruit synovial fibroblasts, osteoclasts and polymorphonuclear neutrophils that release proteases, acids, and ROS such as O₂⁻, resulting in irreversible cartilage and bone destruction.

In the collagen-induced RA animal model, the participation of T and B cells is required to initiate the disease. B cell activation signals through spleen tyrosine kinase (Syk) and phosphoinositide 3-kinase (PI3K) following antigen receptor triggering [Ward S G, Finan P. Isoform-specific phosphoinositide 3-kinase inhibitors as therapeutic agents. Curr Opin Pharmacol. August; 3(4):426-34, (2003)]. After the engagement of antigen receptors on B cells, Syk is phosphorylated on three tyrosines. Syk is a 72-kDa protein-tyrosine kinase that plays a central role in coupling immune recognition receptors to multiple downstream signaling pathways. This function is a property of both its catalytic activity and its ability to participate in interactions with effector proteins containing SH2 domains. Phosphorylation of Tyr-317, -342, and -346 create docking sites for multiple SH2 domain containing proteins, [Hutchcroft, J. E., Harrison, M. L. & Geahlen, R. L. (1992). Association of the 72-kDa protein-tyrosine kinase Ptk72 with the B-cell antigen receptor, J. Biol. Chem, 267: 8613-8619, (1992) and Yamada, T., Taniguchi, T., Yang, C., Yasue, S., Saito, H. & Yamamura, H. Association with B-cell antigen cell antigen receptor with protein-tyrosine kinase-P72(Syk) and activation by engagement of membrane IgM. Eur. J. Biochem. 213: 455-459,(1993)].

Syk has been shown to be required for the activation of PI3K in response to a variety of signals including engagement of the B cell antigen receptor (BCR) and macrophage or neutrophil Fc receptors. [See Crowley, M. T., et al,. J. Exp. Med, 186: 1027-1039, (1997); Raeder, E. M., et at, J. Immunol. 163,6785-6793, (1999); and Jiang, K., et al., Blood 101, 236-244, (2003)]. In B cells, the BCR-stimulated activation of PI3K can be accomplished through the phosphorylation of adaptor proteins such as BCAP, CD19, or Gab1, which creates binding sites for the p85 regulatory subunit of PI3K. Signals transmitted by many IgG receptors require the activities of both Syk and PI3K and their recruitment to the site of the clustered receptor. In neutrophils and monocytes, a direct association of PI3K with phosphorylated immunoreceptor tyrosine based activation motif sequences on FcgRIIA was proposed as a mechanism for the recruitment of PI3K to the receptor. And recently a direct molecular interaction between Syk and PI3K has been reported [Moon K D, et al , Molecular Basis for a Direct Interaction between the Syk Protein-tyrosine Kinase and Phosphoinositide 3-Kinase. J. Biol. Chem. 280, No, 2, Issue of January 14, pp. 1543-1551, (2005)].

The precise mechanisms for the chemopreventive effects of NSAIDs are not yet known, however the ability of these drugs to induce inhibition of cell proliferation, inhibition of angiogenesis, and induction of apoptosis is well known [7 Shiff, S. J., and Rigas, B. (1997) Gastroenterology 113, 1992-1998 and Elder, D. J. E., and Paraskeva, C. (1999) Apoptosis 4, 365-372].

The most characterized target for NSAIDs is cyclooxygenase (COX), which catalyzes the synthesis of prostaglandins from arachidonic acid. There are two known COX isoforms, COX-1 and COX-2, COX-1 is a constitutively expressed enzyme found in most tissues and remains unaltered in colorectal cancer, while COX-2 expression can be up-regulated by a variety of cytokines, hormones, phorbol esters, and oncogenes in colorectal adenomas and adenocarcinomas [Eberhart, C. E., Coffey, R. J., Radhika, A., Giardiello, F. M., Ferrenbach, S., and DuBois, R. N. (1994) Gastroenterology 107, 1183-1188].

The molecular basis of the chemopreventive effects of NSAIDs for colon cancer has been attributed at least in part to inhibition of COX-2 by induction of the susceptibility of cancer cells to apoptosis [Rigas, B., and Shiff, S. J. (2000) Med. Hypotheses 54, 210-215]. A null mutation of COX-2 in a murine model of familial adenomatous polyposis, restored apoptosis and reduced the size and the number of colorectal adenomas [Oshima, M., Dinchuk, J. E., Kargman, S. L., Oshima, H., Hancock, B., Kwong, E., Trzaskos, J. M., Evans, J. F., and Taketo, M. M. (1996) Cell 87, 803-809]. Similar regression of adenomas has been observed by treatment of Min mouse with the NSAID sulindac [Labayle, D., Fischer, D., Vielh, P., Drouhin, F., Pariente, A., Bories, C., Duhamel, O., Trousset, M., and Attali, P. (1991) Gastroenterology 101,635-639].

However, observations relating to the proapoptotic effect of NSAIDs lead to contradictory conclusions and demonstrate that they act via COX-dependent and COX-independent mechanisms [Rigas, B., and Shiff, S. J, (2000) Med. Hypotheses 54, 210-215]. For example, the addition of exogenous prostaglandins to a colon cancer cell line that lacks COX activity cannot reverse the proapoptotic effect of sulindac sulfide, a metabolite derived from sulindac [Hanif, R., Pittas, A., Feng, Y., Koutsos, M. I., Qiao, L., Staiano-Coico, L., Shiff, S. I., and Rigas, B. (1996) Biochem. Pharmacol 52, 237-245].

Also, sulindac sulfone, another sulindac metabolite that does not inhibit COXs, affects tumor growth in animal models [Piazza, G. A., Alberts, D. S., Flixson, L. J., Paranka, N. S., Li, H., Finn, T., Bogert, C., Guillen, J. M., Brendel, K., Gross, P. H., Sperl, G., Ritchie, J., Burt, R. W., Ellsworth, L., Ahnen, D. J., and Pamukcu, R. (1997) CancerRes. 57, 2909-2915] and induces apoptosis in cultured cancer cells expressing or not expressing COXs.

Hence, a wide body of evidence now exists demonstrating that molecular targets of NSAIDs in addition to COX-1 and COX-2 exist and provide a link between the chemoprotective effect of NSAIDs on cancer cells and their level of COX expression. Recent studies have identified a series of new molecular targets for NSAIDS mainly involved in signaling pathways including the extracellular signal-regulated kinase 1/2 signaling [Rice, P. L., Goldberg, R. J., Ray, E. C., Driggers, L. J., and Ahnen, D. J. (2001) Cancer Res. 61, 1541-1547), NF-_B (21. Kopp, E., and Ghosh, S. (1994) Science 265, 956-959), p7056 kinase (Law, B. K., Waltner-Law, M. E., Entingh, A. J., Chytil, A., Aakre, M. E, Norgaard, P., and Moses, H. L. (2000) J. Biol. Chem 275, 38261-38267), p21ras signaling (Herrmann, C., Block, C., Geisen, C., Haas, K., Weber, C., Winde, G., Moroy, T., and Muller, O. (1998) Oncogene 17, 1769-1776), and Akt/PKB kinase (Hsu, A. L., Ching, T. T., Wang, D. S., Song, X., Rangnekar, V. M., and Chen, C. S. (2000) J Biol Chem. 275, 11397-11403]

Much research has shown that inhibitors of COX-2 activity result in decreased production of PGE₂ and are effective in pain relief for patients with chronic arthritic conditions such as RA. However, concern has been raised over the adverse effects of agents that inhibit COX enzyme activity since both COX-1 and COX-2 are involved in important maintenance functions in tissues such as the gastrointestinal and cardiovascular systems. Therefore, designing a safe, long term treatment approach for pain relief in these patients is necessary. Since inducers of COX-2 and iNOS synthesis signal through the Syk, PI3K, p38, ERK1/2, and NF-kB dependent pathways, inhibitors of these pathways may be therapeutic in autoimmune conditions and in particular in the inflamed and degenerating joints of RA patients.

Other kinases currently being investigated for their association with disease symptomology include Aurora, FGFR, MSK, Rse, and Syk.

Aurora—important regulators of cell division, are a family of serine/threonine kinases including Aurora A, B and C. Aurora A and B kinases have been identified to have direct but distinct roles in mitosis. Over-expression of these three isoforms have been linked to a diverse range of human tumor types, including leukemia, colorectal, breast, prostate, pancreatic, melanoma and cervical cancers.

Fibroblast growth factor receptor (FGFR) is a receptor tyrosine kinase. Mutations in this receptor can result in constitutive activation through receptor dimerization, kinase activation, and increased affinity for FGF. FGFR has been implicated in achondroplasia, angiogenesis, and congenital diseases.

MSK (mitogen- and stress-activated protein kinase) 1 and MSK2 are kinases activated downstream of either the ERK (extracellular-signal-regulated kinase) 1/2 or p38 MAPK (mitogen-activated protein kinase) pathways in viva and are required for the phosphorylation of CREB (cAMP response element-binding protein) and histone H3.

Rse is mostly highly expressed in the brain. Rse, also known as Brt, BYK, Dtk, Etk3, Sky, Tif, or sea-related receptor tyrosine kinase, is a receptor tyrosine kinase whose primary role is to protect neurons from apoptosis. Rse, Axl, and Mer belong to a newly identified family of cell adhesion molecule-related receptor tyrosine kinases, GAS6 is a ligand for the tyrosine kinase receptors Rse, Axl, and Mer. GAS6 functions as a physiologic anti-inflammatory agent produced by resting EC and depleted when pro-inflammatory stimuli turn on the pro-adhesive machinery of EC.

Glycogen synthase kinase-3 (GSK-3), present in two isoforms, has been identified as an enzyme involved in the control of glycogen metabolism, and may act as a regulator of cell proliferation and cell death. Unlike many serine-threonine protein kinases, GSK-3 is constitutively active and becomes inhibited in response to insulin or growth factors. Its role in the insulin stimulation of muscle glycogen synthesis makes it an attractive target for therapeutic intervention in diabetes and metabolic syndrome,

GSK-3 dysregulation has been shown to be a focal point in the development of insulin resistance. Inhibition of GSK3 improves insulin sensitivity not only by an increase of glucose disposal rate but also by inhibition of gluconeogenic genes such as phosphoenolpyruvate carboxykinase and glucose-6-phosphatase in hepatocytes. Furthermore, selective GSK3 inhibitors potentiate insulin-dependent activation of glucose transport and utilization in muscle in vitro and in vivo. GSK3 also directly phosphorylates serine/threonine residues of insulin receptor substrate-1, which leads to impairment of insulin signaling. GSK3 plays an important role in the insulin signaling pathway and it phosphorylates and inhibits glycogen synthase in the absence of insulin [Parker, P. J., Caudwell, F. B., and Cohen, P. (1983) Eur. J Biochem 130:227-234]. Increasing evidence supports a negative role of GSK-3 in the regulation of skeletal muscle glucose transport activity. For example, acute treatment of insulin-resistant rodents with selective GSK-3 inhibitors improves whole-body insulin sensitivity and insulin action on muscle glucose transport. Chronic treatment of insulin-resistant, pre-diabetic obese Zucker rats with a specific GSK-3 inhibitor enhances oral glucose tolerance and whole-body insulin sensitivity, and is associated with an amelioration of dyslipidemia and an improvement in IRS-1-dependent insulin signaling in skeletal muscle. These results provide evidence that selective targeting of GSK-3 in muscle may be an effective intervention for the treatment of obesity-associated insulin resistance.

Syk is a non-receptor tyrosine kinase related to ZAP-70 that is involved in signaling from the B-cell receptor and the IgE receptor, Syk binds to ITAM motifs within these receptors, and initiates signaling through the Ras, PI3K, and PLCg signaling pathways, Syk plays a critical role in intracellular signaling and thus is an important target for inflammatory diseases and respiratory disorders.

Angiogenesis is the process of vascularization of a tissue involving the development of new capillary blood vessels. The regulation and control of angiogenesis is important to numerous disease states associated with such ocular disorders as macular degeneration or diabetic retinopathy. Additionally, angiogenesis is a key component for successful metastatic cancer dissemination and survival.

A number of protein kinases have been implicated in the angiogenic process. For example, recent work has identified the PI3K-Akt-PTEN signaling node as an intercept point for the control of angiogenesis in brain tumors [Castellino R C and Durden D L., Mechanisms of Disease: the PI3K-Akt-PTEN signaling node-an intercept point for the control of angiogenesis in brain tumors. Nat Clin Pract Neural. 3(12):682-93, 2007] See also [Blackburn J S, et al., RNA interference inhibition of matrix metalloproteinase-1 prevents melanoma metastasis by reducing tumor collagenase activity and angiogenesis, Cancer Res. 67(22):10849-58 2007]. Additionally, for example, Lee and colleagues have demonstrated the relation of AKT angiogenesis in a human gastric colon cancer model [Lee, B L., et al., A hypoxia-independent up regulation of hypoxia-inducible factor-1 by Akt contributes to angiogenesis in human gastric cancer. Carcinogenesis. 2007 Nov. 4.

Therefore, it would be useful to identify methods and compositions that would modulate the expression or activity of single or multiple selected kinases. The realization of the complexity of the relationship and interaction among and between the various protein kinases and kinase pathways reinforces the pressing need for developing pharmaceutical agents capable of acting as protein kinase modulators, regulators or inhibitors that have beneficial activity on multiple kinases or multiple kinase pathways. A single agent approach that specifically targets one kinase or one kinase pathway may be inadequate to treat very complex diseases, conditions and disorders, such as, for example, diabetes and metabolic syndrome. Modulating the activity of multiple kinases may additionally generate synergistic therapeutic effects not obtainable through single kinase modulation.

Such modulation and use may require continual use for chronic conditions or intermittent use, as needed for example in inflammation, either as a condition unto itself or as an integral component of many diseases and conditions. Additionally, compositions that act as modulators of kinase can affect a wide variety of disorders in a mammalian body. I

Currently, there is a trend favoring the development of multi-targeted treatment modalities for disease conditions thereby providing the potential for enhanced responsiveness with a concommitant potential to reduce the potential toxicities associated with aggressive treatment agains a single target. See [Arbiser, J L., Why targeted therapy hasn't worked in advanced cancer., J. Clin Invest., 117(10): 2762-65, 2007, and Ma, W W and Hildalgo, M., Exploiting novel molecular targets in gastrointestinal cancers. World J Gastroenterol. 13(44): 5845-56,2007] The instant invention describes substituted 1,3-cyclopentadione compounds that may be used to regulate the activity of multiple kinases, thereby providing a means to treat numerous disease related symptoms with a concomitant increase in the quality of life.

### SUMMARY OF THE INVENTION

The present invention relates generally to methods and compositions that can be used to treat or inhibit angiogenesis, cancers and their associated inflammatory pathways susceptible to protein kinase modulation. More specifically, the invention relates to methods and compositions that utilize substituted 1,3-cyclopentadione compounds.

A first embodiment of the invention describes methods to treat a cancer responsive to protein kinase modulation in a mammal in need. The method comprises administering to the mammal a therapeutically effective amount of a substituted 1,3-cyclopentadione compound.

A second embodiment of the invention describes compositions to treat a cancer responsive to protein kinase modulation in a mammal in need where the composition comprises a therapeutically effective amount of a substituted 1,3-cyclopentadione compound where the therapeutically effective amount modulates a cancer associated protein kinase.

A third embodiment of the invention describes methods to treat angiogenic conditions responsive to protein kinase modulation in a mammal in need. The method comprises administering to the mammal a therapeutically effective amount of a substituted 1,3-cyclopentadione compound.

A further embodiment of the invention describes compositions to treat angiogenic conditions responsive to protein kinase modulation in a mammal in need where the composition comprises a therapeutically effective amount of a substituted 1,3-cyclopentadione compound where the therapeutically effective amount modulates an angiogenic associated protein kinase.

Another embodiment describes methods to modulate inflammation associated with cancer or angiogenesis. The method comprises administering to the mammal a therapeutically effective amount of a substituted 1,3-cyclopentadione compound

Compositions for treating inflammation associated with angiogenesis or cancer are described in another embodiment of the invention. Here the compositions comprise a therapeutically effective amount of a substituted 1,3-cyclopentadione compound where the therapeutically effective amount modulates inflammation associated protein kinases.

In one embodiment, the invention describes a composition to treat a cancer or angiogenic conditions responsive to protein kinase modulation in a mammal in need thereof, said composition includes a therapeutically effective amount of a cis-n-tetrahydro-isoalpha acid (TH5) as the only substituted 1,3-cyclopentadione compound in the composition; wherein said therapeutically effective amount modulates a cancer associated protein kinase or an angiogenesis associated protein kinase.

In one embodiment, the invention describes a composition to treat a cancer or angiogenic conditions responsive to protein kinase modulation in a mammal in need thereof, said composition consisting essentially of therapeutically effective amounts of one or more (n) analogs of substituted 1,3-cyclopentadione compound and optionally one or more (ad) analogs of substituted 1,3-cyclopentadione compound in the composition; wherein said therapeutically effective amount modulates a cancer associated protein kinase or an angiogenesis associated protein kinase.

In one embodiment, the invention describes a composition to treat a cancer or angiogenic conditions responsive to protein kinase modulation in a mammal in need thereof, said composition consisting essentially of therapeutically effective amount of one or more (co) analogs of substituted 1,3-cyclopentadione compound in the composition; wherein said therapeutically effective amount modulates a cancer associated protein kinase or an angiogenesis associated protein kinase.

In one embodiment, the invention describes a composition to treat a cancer or angiogenic conditions responsive to protein kinase modulation in a mammal in need thereof, said composition incldues a therapeutically effective amount of only one analog of a substituted 1,3-cyclopentadione compound; wherein said therapeutically effective amount modulates a cancer associated protein kinase or an angiogenesis associated protein kinase.

In one embodiment, the invention describes a composition to treat a cancer or angiogenic conditions responsive to protein kinase modulation in a mammal in need thereof, said composition includes one or more of the substituted 1,3-cyclopentadione compounds selected from the group consisting of rho (6S) cis n iso-alpha acid, rho (6S) cis n iso-alpha acid, rho (6R) cis n iso-alpha acid, rho (6R) trans n iso-alpha acid, rho (6S) trans n iso-alpha acid, rho (6R) cis rho n iso-alpha acid, rho (6S) cis n iso-alpha acid, (6S) trans rho n iso-alpha acid, rho (6R) trans n iso-alpha acid, rho (6S) cis co iso-alpha acid, rho (6R) cis co iso-alpha acid, rho (6R) trans co iso-alpha acid, rho (6S) trans co iso-alpha acid, rho (6R) cis co iso-alpha acid, rho (6S) cis co iso-alpha acid, rho (6S) trans co iso-alpha acid, rho (6R) trans co iso-alpha acid, rho (6S) cis ad iso-alpha acid, rho (6R) cis ad iso-alpha acid, rho (6R) trans ad iso-alpha acid, rho (6S) trans ad iso-alpha acid, rho (6R) cis ad iso-alpha acid, rho (6S) cis ad iso-alpha acid, rho (6S) trans ad iso-alpha acid, rho (6R) trans ad iso-alpha acid, tetrahydro cis n iso-alpha acid, tetrahydro trans n iso-alpha acid, tetrahydro cis n iso-alpha acid, tetrahydro trans n iso-alpha acid, tetrahydro cis co iso-alpha acid, tetrahydro trans co iso-alpha acid, tetrahydro cis co iso-alpha acid, tetrahydro trans co iso-alpha acid, tetrahydro cis ad iso-alpha acid, tetrahydro trans ad iso-alpha acid, tetrahydro cis ad iso-alpha acid, tetrahydro trans ad iso-alpha acid, hexahydro (6S) cis n iso-alpha acid, hexahydro (6R) cis n iso-alpha acid, hexahydro (6R) trans n iso-alpha acid, hexahydro (6S) trans n iso-alpha acid, hexahydro (6R) cis n iso-alpha acid, hexahydro (6S) cis n iso-alpha acid, hexahydro (6S) trans n iso-alpha acid, hexahydro (6R) trans n iso-alpha acid, hexahydro (6S) cis co iso-alpha acid, hexahydro (6R) cis co iso-alpha acid, hexahydro (6R) trans co iso-alpha acid, hexahydro (6S) trans co iso-alpha acid, hexahydro (6R) cis co iso-alpha acid, hexahydro (6S) cis co iso-alpha acid, hexahydro (6S) trans co iso-alpha acid, hexahydro (6R) trans co iso-alpha acid, hexahydro (6S) cis ad iso-alpha acid, hexahydro (6R) cis ad iso-alpha acid, hexahydro (6R) trans ad iso-alpha acid, hexahydro (6S) trans ad iso-alpha acid, hexahydro (6R) cis ad iso-alpha acid, hexahydro (6S) cis ad iso-alpha acid, hexahydro (6S) trans ad iso-alpha acid, hexahydro (6R) trans ad iso-alpha acid, lupolone, colupulone, adlupulone, prelupulone, postlupulone, and xanthohumol.

### BRIEF DESCRIPTION OF THE DRAWINGS

FIG. 1 graphically depicts a portion of the kinase network regulating NF-κB in relation to cancer, angiogenesis and inflammation.

FIG. 2 depicts the chemical structure of individual members forming Meta-THc.

FIG. 3 depicts a representative chromatogram of a Meta-THc composition. The top panel identifies the chromatagraphic peaks comprising the Meta-THc components of the mixture whereas the subsequent panels describe the chromatagraphic profile of the isolation fractions comprising the peaks.

FIG. 4 depicts the inhibitory effects of Meta-THc on PI3K and assorted kinases associated with cancer, angiogenesis, and inflammation.

FIG. 5 provides a graphic representation of the inhibition of PGE₂ and nitric oxide production in LPS activated RAW 264.7 cells by Meta-THc.

FIG. 6 provides a graphic representation of the inhibition of COX-2 protein expression in RAW 2643 cells by Meta-THc.

FIG. 7 provides a graphic representation demonstrating that Meta-THc did not inhibit PGE₂ production by preformed COX-2 LPS activated RAW 2643 cells.

FIG. 8 provides a representative Western blot analysis showing inhibition by Meta-THc of NF-κB binding in LPS activated RAW 264.7 cell nuclear extract,

FIG. 9 graphically depicts the inhibition by Meta-THc of TNFα and IL1-β induced MMP-13 expression in the SW1353 human chondrosarcoma cell line

FIG. 10 graphically displays the inhibitory effects of Meta-THc analogs on PGE₂ and nitric oxide production in LPS activated RAW 264.7 cells.

FIG. 11 provides a graphic representation depicting the inhibitory effect of Meta-THc analogs on MAPK1 kinase.

FIG. 12 is a graphic representation depicting the inhibitory effect of Meta-THc analogs on a panel of inflammation associated kinases.

FIG. 13 provides a graphic representation depicting the inhibitory effect of Meta-THc analogs on GSK kinase.

FIG. 14 is a graphic representation of the effect of Meta-THc analogs on the angiogenesis associated kinase Arg Tyrosine kinase.

FIG. 15 depicts the effects of Meta-THc analogs on a panel of kinases involved in colon cancer progression.

FIG. 16 graphically depicts the effects of Meta-THc on the arthritic index in a murine model of rheumatoid arthritis.

FIG. 17 graphically depicts the effects of Meta-THc analogs on the growth of HT-29, Caco-2 and SW480 colon cancer cell lines.

FIG. 18 graphically displays the detection of Meta-THc in the serum over time following ingestion of 940 mg of Meta-THc in humans.

FIG. 19 displays the profile of Meta-THc detectable in the serum versus control,

FIG. 20 depicts the metabolism of Meta-THc by CYP2C9*1.

FIG. 21 depicts chemical structures of beta acids: lupulone, colupulone, adlupulone, prelupulone and postlupuline.

FIG. 22 depicts the chemical structure of xanthohumol.

FIG. 23 shows the gini coefficients for different THs (tetrahydroisoalpha acids).

FIG. 24 shows a comparison between the Gini coefficients of TH1-7 and other kinase drugs on over 200 human protein kinases.

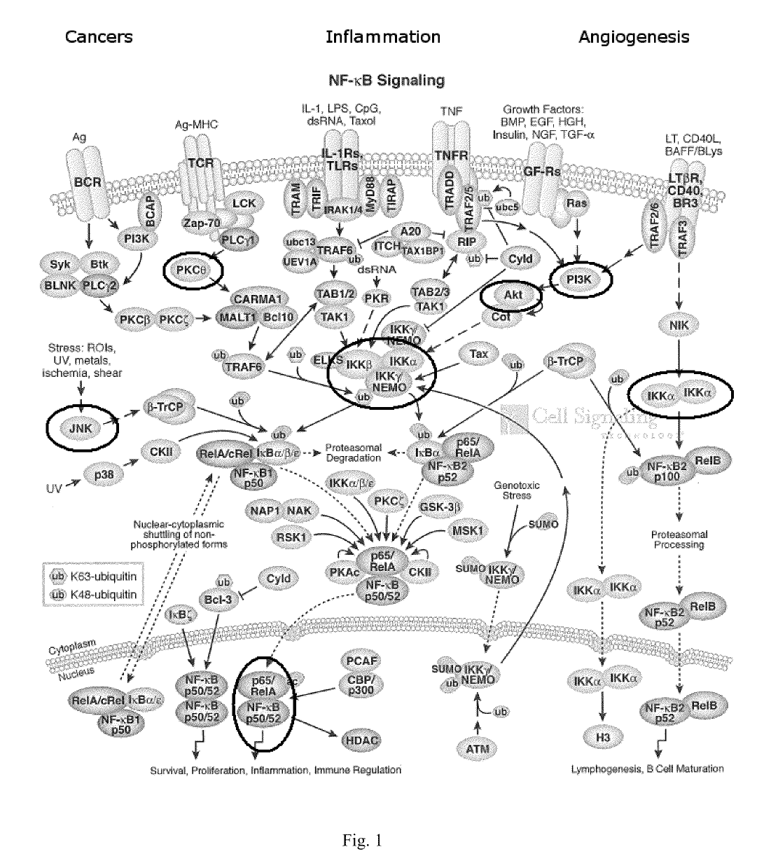

### DETAILED DESCRIPTION OF THE INVENTION

The present invention relates generally to methods and compositions that are used to treat or inhibit angiogenesis, cancers and their associated inflammatory pathways susceptible to protein kinase modulation. More specifically, the invention relates to methods and compositions that utilize substituted 1,3-cyclopentadione compounds.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006)

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables that are inherently discrete, and can be 0.0, 0.1, 0.01, 0.001, or any other real value for variables that are inherently continuous.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

As used herein, “disease associated kinase” means those individual protein kinases or groups or families of kinases that are either directly causative of the disease or whose activation is associated with pathways that serve to exacerbate the symptoms of the disease in question.

The phrase “protein kinase modulation is beneficial to the health of the subject” refers to those instances wherein the kinase modulation (either up or down regulation) results in reducing, preventing, and/or reversing the symptoms of the disease or augments the activity of a secondary treatment modality.

The phrase “a cancer responsive to protein kinase modulation” refers to those instances where administration of the compounds of the invention either a) directly modulates a kinase in the cancer cell where that modulation results in an effect beneficial to the health of the subject (e.g., apoptosis or growth inhibition of the target cancer cell; b) modulates a secondary kinase wherein that modulation cascades or feeds into the modulation of a kinase that produces an effect beneficial to the health of the subject; or c) the target kinases modulated render the cancer cell more susceptible to secondary treatment modalities (e.g., chemotherapy or radiation therapy).

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or compounds, but may also include additional features or compounds.

As used herein, the term “substituted 1,3-cyclopentadione compound” refers to a compound selected from the group consisting of dihydro-(Rho) isoalpha acids; tetra-hydroisoalpha acids; hexa-hydroisoalpha acids; beta acids; xanthohumol; their individual analogs; and mixtures thereof. A substituted 1,3-cyclopentadione compound can be chemically synthesized de novo or extracted or derived from a natural source (e.g., hop or hop compounds).

As used herein, the terms “derivatives” or a matter “derived” refer to a chemical substance related structurally to another substance and theoretically obtainable from it, i.e. a substance that can be made from another substance. Derivatives can include compounds obtained via a chemical reaction.

As used herein, “dihydro-isoalpha acid” or “Rho-isoalpha acid” refers to analogs of Rho-isoalpha acid—including cis and trans forms of the isohumulone (n-), isocohumulone (co-) and isadhumulone (ad-) analogs—as depicted in Table 1 or a mixture thereof. Rho-isoalpha acid, for example, refers to a mixture of one or more of dihydro-isohumulone, dihydro-isocohumulone, dihydro-adhumulone.

As used herein, “tetrahydro-isoalpha acid” or “Meta-THc” refers to analogs of tetrahydro-isoalpha acid—including cis and trans forms of the isohumulone (n-), isocohumulone (co-) and isadhumulone (ad-) analogs—as depicted in Table 2 or a mixture thereof. Tetrahydro-isoalpha acid or Meta-THc, for example, refers to a mixture of one or more of tetrahydro-adhumulone, tetrahydro-isocohumulone, tetrahydro-isohumulone.

As used herein, “hexahydro-isoalpha acid” refers to analogs of hexahydro-isoalpha acid—including cis and trans forms of the isohumulone (n-), isocohumulone (co-) and isadhumulone (ad-) analogs—as depicted in Table 3 or a mixture thereof. Hexahydro-isoalpha acid, for example, refers to a mixture of one or more of hexahydro-isohumulone, hexahydro-isocohumulone, hexahydro-adhumulone.

As used herein “beta acid” refers to any mixture of one or more of lupulone, colupulone, adlupulone, prelupulone, postlupuline or analogs thereof.

As used herein, “tetrahydro-isohumulone” shall further refer to the cis and trans forms of (+)-(4R,5S)-3,4-dihydroxy-2-(3-methylbutanoyl)-5-(3-methylbutyl)-4-(4-methylpentanoyl)cyclopent-2-en-1-one, (−)-(4S,5S)-3,4-dihydroxy-2-(3-methylbutanoyl)-5-(3-methylbutyl)-4-(4-methylpentanoyl)cyclopent-2-en-1-one respectively, or (n-) compounds shown in Table 2.

“Tetrahydro-isocohumulone”, as used herein refers to the cis and trans forms of (+)-(4R,5S)-3,4-dihydroxy-5-(3-methylbutyl)-4-(4-methylpentanoyl)-2-(3-methylpropanoyl)cyclopent-2-en-1-one, (−)-(4S,5S)-3,4-dihydroxy-5-(3-methylbutyl)-4-(4-methylpentanoyl)-2-(3-methylpropanoyl)cyclopent-2-en-1-one respectively, or (co-) compounds shown in Table 2.

“Tetrahydro-adhumulone” shall be used herein to refer to the cis and trans forms of (+)-(4R,5S)-3,4-dihydroxy-2-(2-methylbutanoyl)-5-(3-methylbutyl)-4-(4-methylpentanoyl)cyclopent-2-en-1-one and (+)-(4R,5S)-3,4-dihydroxy-5-(3-methylbutyl)-4-(4-methylpentanoyl)-2-petanoylcyclopent-2-en-1-one respectively, or (ad-) compounds shown in Table 2.

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

Compounds according to the invention may be present as salts. In particular, pharmaceutically acceptable salts of the compounds are contemplated. A “pharmaceutically acceptable salt” of the invention is a combination of a compound of the invention and either an acid or a base that forms a salt (such as, for example, the magnesium salt, denoted herein as “Mg” or “Mag”) with the compound and is tolerated by a subject under therapeutic conditions. In general, a pharmaceutically acceptable salt of a compound of the invention will have a therapeutic index (the ratio of the lowest toxic dose to the lowest therapeutically effective dose) of 1 or greater. The person skilled in the art will recognize that the lowest therapeutically effective dose will vary from subject to subject and from indication to indication, and will thus adjust accordingly.

The compounds according to the invention are optionally formulated in a pharmaceutically acceptable vehicle with any of the well known pharmaceutically acceptable carriers, including diluents and excipients [see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995]. While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well as any other pharmacologically active ingredient useful for the treatment of the symptom/condition being treated.

TABLE 1
Rho dihydro-isoalpha acids

| Chemical Name | Synonym | Structure |
|---|---|---|
| (4S,5S)-3,4-dihydroxy-4-[(1S)-hydroxy-4- methylpent-3-en-1-yl]-2-(3- methylbutanoyl)-5-(3-methylbut-2-en-1- yl)cyclopent-2-en-1-one | rho (6R) cis n iso-alpha acid | 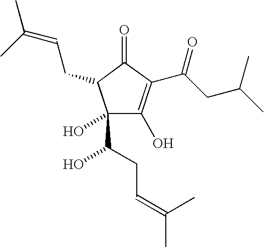 |
| (4S,5S)-3,4-dihydroxy-4-[(1R)-hydroxy-4- methylpent-3-en-1-yl]-2-(3- methylbutanoyl)-5-(3-methylbut-2-en-1- yl)cyclopent-2-en-1-one | rho (6R) cis n iso-alpha acid | 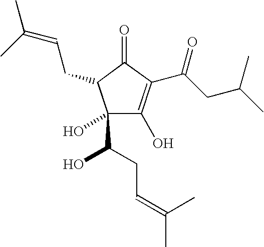 |
| (4R,5S)-3,4-dihydroxy-4-[(1R)-hydroxy-4- methylpent-3-en-1-yl]-2-(3- methylbutanoyl)-5-(3-methylbut-2-en-1- yl)cyclopent-2-en-1-one | rho (6S) trans n iso-alpha acid | 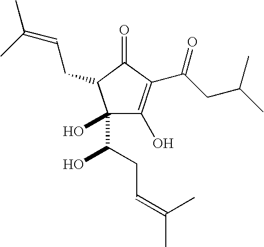 |
| (4R,5S)-3,4-dihydroxy-4-[(1S)-hydroxy-4- methylpent-3-en-1-yl]-2-(3- methylbutanoyl)-5-(3-methylbut-2-en-1- yl)cyclopent-2-en-1-one | rho (6R) trans n iso-alpha acid | 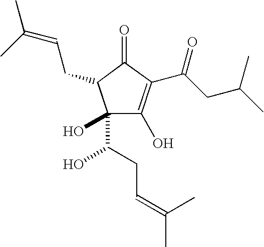 |
| (4R,5R)-3,4-dihydroxy-4-[(1R)-hydroxy-4- methylpent-3-en-1-yl]-2-(3- methylbutanoyl)-5-(3-methylbut-2-en-1- yl)cyclopent-2-en-1-one | rho (6R) cis rho n iso-alpha acid | 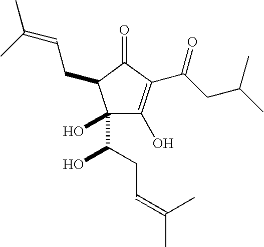 |
| (4R,5R)-3,4-dihydroxy-4-[(1S)-hydroxy-4- methylpent-3-en-1-yl]-2-(3- methylbutanoyl)-5-(3-methylbut-2-en-1- yl)cyclopent-2-en-1-one | rho (6S) cis n iso-alpha acid | 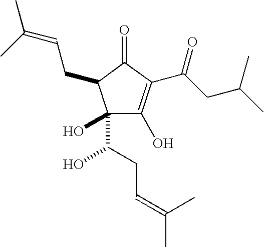 |
| (4S,5R)-3,4-dihydroxy-4-[(1S)-hydroxy-4- methylpent-3-en-1-yl]-2-(3- methylbutanoyl)-5-(3-methylbut-2-en-1- yl)cyclopent-2-en-1-one | (6S) trans rho n iso-alpha acid | 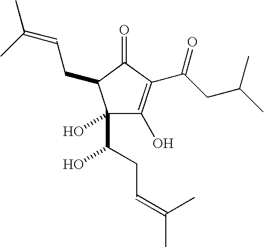 |
| (4S,5R)-3,4-dihydroxy-4-[(1R)-hydroxy-4- methylpent-3-en-1-yl]-2-(3- methylbutanoyl)-5-(3-methylbut-2-en-1- yl)cyclopent-2-en-1-one | rho (6R) trans n iso-alpha acid | 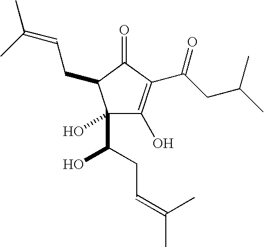 |
| (4S,5S)-3,4-dihydroxy-4-[(1S)-hydroxy-4- methylpent-3-en-1-yl]-5-(3-methylbut-2- en-1-yl)-2-(2-methylpropanoyl)cyclopent- 2-en-1-one | rho (6S) cis co iso-alpha acid | 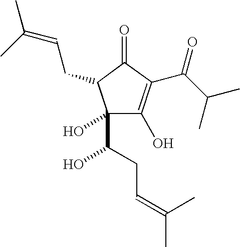 |
| (4S,5S)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | rho (6R) cis co iso-alpha acid | 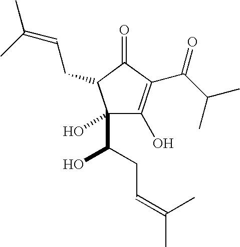 |
| (4R,5S)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | rho (6R) trans co iso-alpha acid | 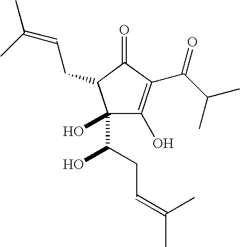 |
| (4R,5S)-3,4-dihydroxy-4-[(1S)-hydroxy-4- methylpent-3-en-1-yl]-5-(3-methylbut-2- en-1-yl)-2-(2-methylpropanoyl)cyclopent- 2-en-1-one | rho (6S) trans co iso-alpha acid | 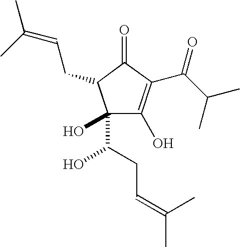 |
| (4R,5R)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | rho (6R) cis co iso-alpha acid | 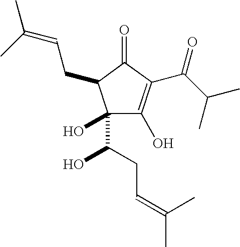 |
| (4R,5R)-3,4-dihydroxy-4-[(1S)-hydroxy-4- methylpent-3-en-1-yl]-5-(3-methylbut-2- en-1-yl)-2-(2-methylpropanoyl)cyclopent- 2-en-1-one | rho (6S) cis co iso-alpha acid | 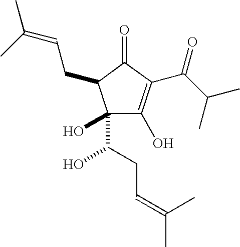 |
| (4S,5R)-3,4-dihydroxy-4-[(1S)-hydroxy-4- methylpent-3-en-1-yl]-2-(2- methylpropanoyl)-5-(3-methylbut-2-en-1- yl)cyclopent-2-en-1-one | rho (6S) trans co iso-alpha acid | 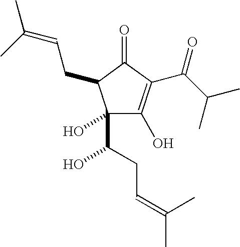 |
| (4S,5R)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | rho (6R) trans co iso-alpha acid | 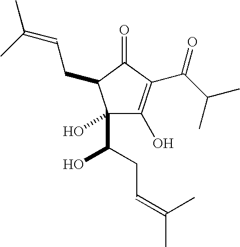 |
| (4S,5S)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | rho (6S) cis ad iso-alpha acid | 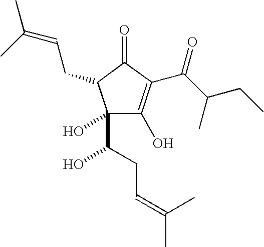 |
| (4S,5S)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | rho (6R) cis ad iso-alpha acid | 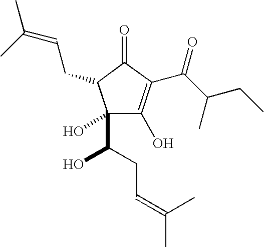 |
| (4R,5S)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | rho (6R) trans ad iso-alpha acid | 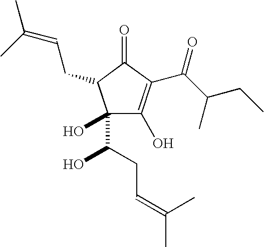 |
| (4R,5S)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | rho (6S) trans ad iso-alpha acid | 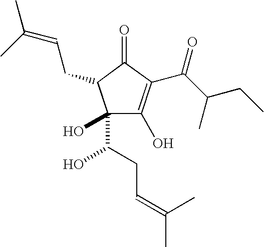 |
| (4R,5R)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | rho (6R) cis ad iso-alpha acid | 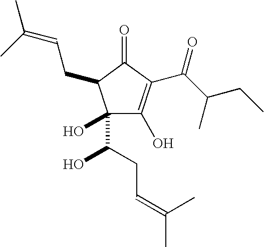 |
| (4R,5R)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | rho (6S) cis ad iso-alpha acid | 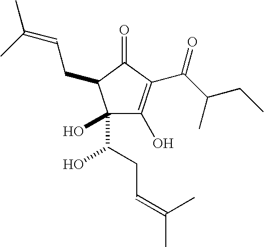 |
| (4S,5R)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | rho (6S) trans ad iso-alpha acid | 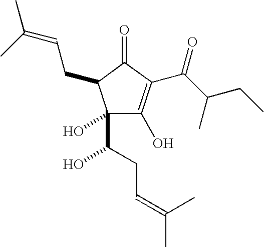 |
| (4S,5R)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | rho (6R) trans ad iso-alpha acid | 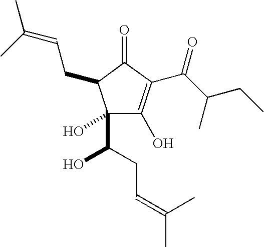 |

TABLE 2
Tetrahydro-isoalpha acids

| Chemical Name | Synonym | Structure |
|---|---|---|
| (4R,5S)-3,4-dihydroxy-2-(3- methylbutanoyl)-5-(3-methylbutyl)-4-(4- methylpentanoyl)cyclopent-2-en-1-one | tetrahydro cis n iso-alpha acid | 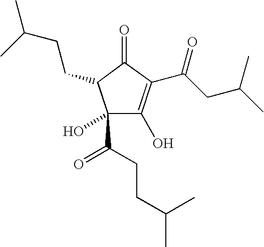 |
| (4S,5S)-3,4-dihydroxy-2-(3- methylbutanoyl)-5-(3-methylbutyl)-4-(4- methylpentanoyl)cyclopent-2-en-1-one | tetrahydro trans n iso-alpha acid | 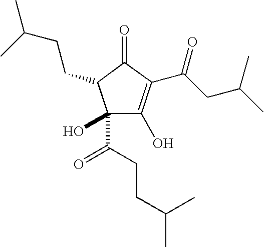 |
| (4S,5R)-3,4-dihydroxy-2-(3- methylbutanoyl)-5-(3-methylbutyl)-4-(4- methylpentanoyl)cyclopent-2-en-1-one | tetrahydro cis n iso-alpha acid | 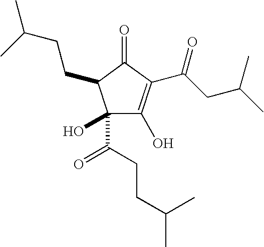 |
| (4R,5R)-3,4-dihydroxy-2-(3- methylbutanoyl)-5-(3-methylbutyl)-4-(4- methylpentanoyl)cyclopent-2-en-1-one | tetrahydro trans n iso-alpha acid | 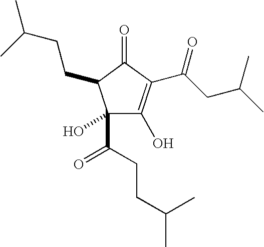 |
| (4R,5S)-3,4-dihydroxy-5-(3-methylbutyl)-4- (4-methylpentanoyl)-2-(3- methylpropanoyl)cyclopent-2-en-1-one | tetrahydro cis co iso-alpha acid | 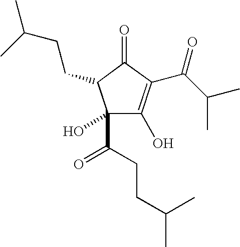 |
| (4S,5S)-3,4-dihydroxy-5-(3-methylbutyl)-4- (4-methylpentanoyl)-2-(3- methylpropanoyl)cyclopent-2-en-1-one | tetrahydro trans co iso-alpha acid | 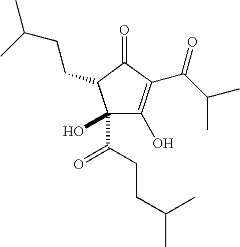 |
| (4S,5R)-3,4-dihydroxy-5-(3-methylbutyl)-4- (4-methylpentanoyl)-2-(3- methylpropanoyl)cyclopent-2-en-1-one | tetrahydro cis co iso-alpha acid | 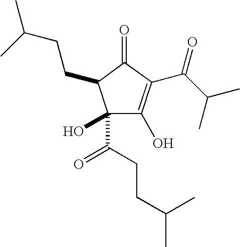 |
| (4R,5R)-3,4-dihydroxy-5-(3-methylbutyl)-4- (4-methylpentanoyl)-2-(3- methylpropanoyl)cyclopent-2-en-1-one | tetrahydro trans co iso-alpha acid | 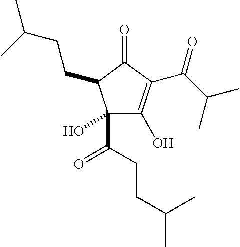 |
| (4R,5S)-3,4-dihydroxy-2-(2- methylbutanoyl)-5-(3-methylbutyl)-4-(4- methylpentanoyl)cyclopent-2-en-1-one | tetrahydro cis ad iso-alpha acid | 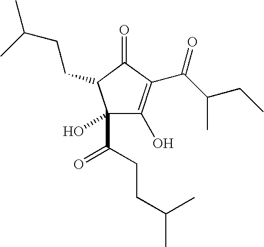 |
| (4S,5S)-3,4-dihydroxy-2-(2- methylbutanoyl)-5-(3-methylbutyl)-4-(4- methylpentanoyl)cyclopent-2-en-1-one | tetrahydro trans ad iso-alpha acid | 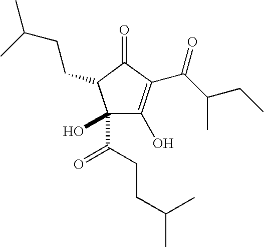 |
| (4S,SR)-3,4-dihydroxy-2-(2- methylbutanoyl)-5-(3-methylbutyl)-4-(4- methylpentanoyl)cyclopent-2-en-1-one | tetrahydro cis ad iso-alpha acid | 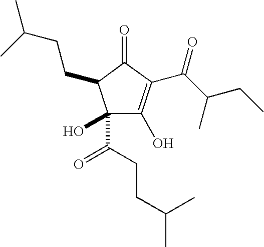 |
| (4R,5R)-3,4-dihydroxy-2-(2- methylbutanoyl)-5-(3-methylbutyl)-4-(4- methylpentanoyl)cyclopent-2-en-1-one | tetrahydro trans ad iso-alpha acid | 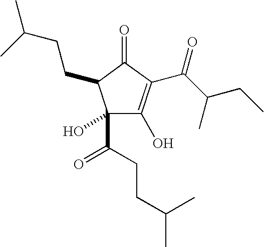 |

TABLE 3
Hexahydro-isoalpha acids

| Chemical Name | Synonym | Structure |
|---|---|---|
| (4S,5S)-3,4-dihydroxy-4-[(1S)-1-hydroxy- 4-methylpentyl]-2-(3-methylbutanoyl)-5- (3-methylbutyl)cyclopent-2-en-1-one | hexahydro (6S) cis n iso-alpha acid | 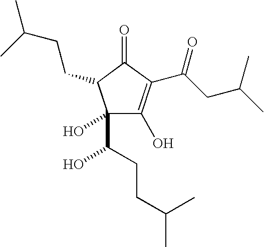 |
| (4S,5S)-3,4-dihydroxy-4-[(1R)-1-hydroxy- 4-methylpentyl]-2-(3-methylbutanoyl)-5- (3-methylbutyl)cyclopent-2-en-1-one | hexahydro (6R) cis n iso-alpha acid | 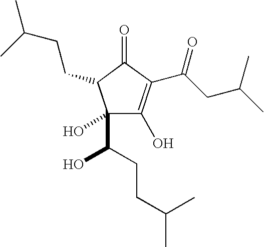 |
| (4R,5S)-3,4-dihydroxy-4-[(1R)-1-hydroxy- 4-methylpentyl]-2-(3-methylbutanoyl)-5- (3-methylbutyl)cyclopent-2-en-1-one | hexahydro (6R) trans n iso-alpha acid | 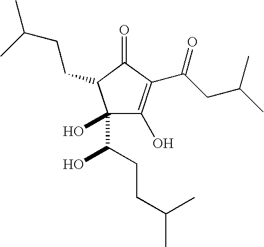 |
| (4R,5S)-3,4-dihydroxy-4-[(1S)-1-hydroxy- 4-methylpentyl]-2-(3-methylbutanoyl)-5- (3-methylbutyl)cyclopent-2-en-1-one | hexahydro (6S) trans n iso-alpha acid | 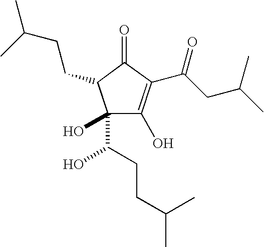 |
| (4R,5R)-3,4-dihydroxy-4-[(1R)-1-hydroxy- 4-methylpentyl]-2-(3-methylbutanoyl)-5- (3-methylbutyl)cyclopent-2-en-1-one | hexahydro (6R) cis n iso-alpha acid | 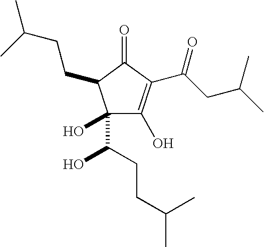 |
| (4R,5R)-3,4-dihydroxy-4-[(1S)-1-hydroxy- 4-methylpentyl]-2-(3-methylbutanoyl)-5- (3-methylbutyl)cyclopent-2-en-1-one | hexahydro (6S) cis n iso-alpha acid | 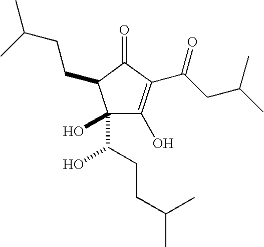 |
| (4S,5R)-3,4-dihydroxy-4-[(1S)-1-hydroxy- 4-methylpentyl]-2-(3-methylbutanoyl)-5- (3-methylbutyl)cyclopent-2-en-1-one | hexahydro (6S) trans n iso-alpha acid | 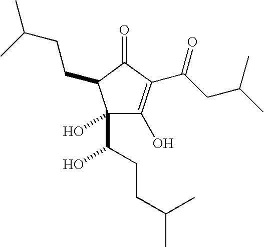 |
| (4S,5R)-3,4-dihydroxy-4-[(1R)-1-hydroxy- 4-methylpentyl]-2-(3-methylbutanoyl)-5- (3-methylbutyl)cyclopent-2-en-1-one | hexahydro (6R) trans n iso-alpha acid | 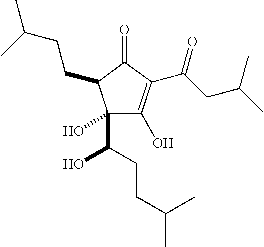 |
| (4S,5S)-3,4-dihydroxy-4-[(1S)-hydroxy-4- methylpent-3-en-1-yl]-5-(3-methylbut-2- en-1-yl)-2-(2-methylpropanoyl)cyclopent- 2-en-1-one | hexahydro (6S) cis co iso-alpha acid | 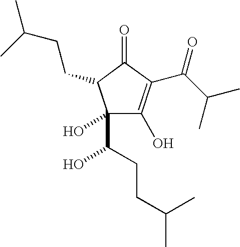 |
| (4S,5S)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | hexahydro (6R) cis co iso-alpha acid | 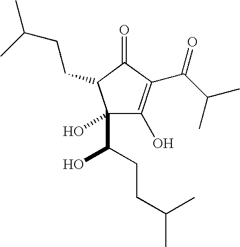 |
| (4R,5S)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | hexahydro (6R) trans co iso- alpha acid | 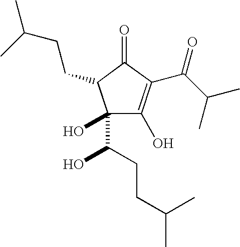 |
| (4R,5S)-3,4-dihydroxy-4-[(1S)-hydroxy-4- methylpent-3-en-1-yl]-5-(3-methylbut-2- en-1-yl)-2-(2-methylpropanoyl)cyclopent- 2-en-1-one | hexahydro (6S) trans co iso- alpha acid | 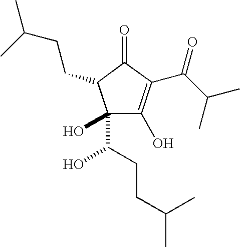 |
| (4R,5R)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | hexahydro (6R) cis co iso-alpha acid | 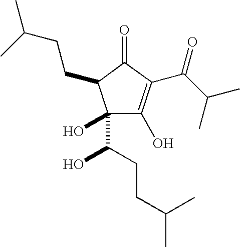 |
| (4R,5R)-3,4-dihydroxy-4-[(1S)-hydroxy-4- methylpent-3-en-1-yl]-5-(3-methylbut-2- en-1-yl)-2-(2-methylpropanoyl)cyclopent- 2-en-1-one | hexahydro (6S) cis co iso-alpha acid | 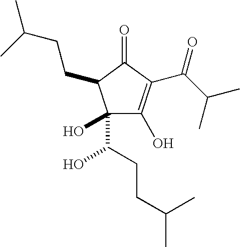 |
| (4S,5R)-3,4-dihydroxy-4-[(1S)-hydroxy-4- methylpent-3-en-1-yl]-2-(2- methylpropanoyl)-5-(3-methylbut-2-en-1- yl)cyclopent-2-en-1-one | hexahydro (6S) trans co iso- alpha acid | 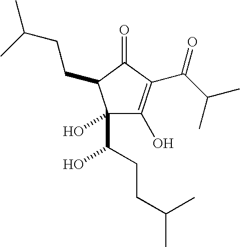 |
| (4S,5R)-3,4-dihydroxy-4-[(1R)-hydroxy-4- methylpent-3-en-1-yl]-5-(3-methylbut-2- en-1-yl)-2-(2-methylpropanoyl)cyclopent- 2-en-1-one | hexahydro (6R) trans co iso- alpha acid | 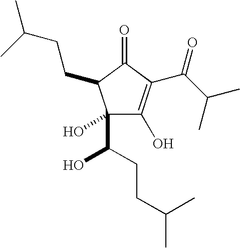 |
| (4S,5S)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | hexahydro (6S) cis ad iso-alpha acid | 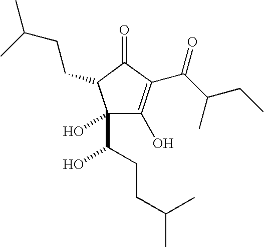 |
| (4S,5S)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | hexahydro (6R) cis ad iso-alpha acid | 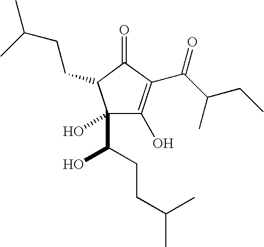 |
| (4R,5S)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | hexahydro (6R) trans ad iso- alpha acid | 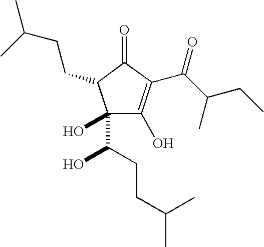 |
| (4R,5S)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | hexahydro (6S) trans ad iso- alpha acid | 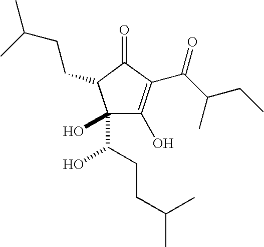 |
| (4R,5R)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | hexahydro (6R) cis ad iso-alpha acid | 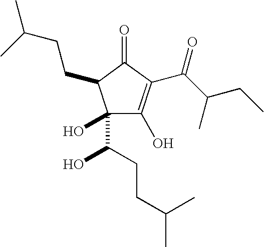 |
| (4R,5R)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | hexahydro (6S) cis ad iso-alpha acid | 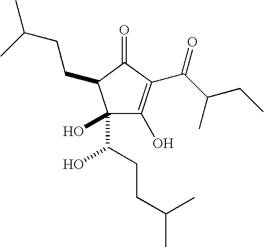 |
| (4S,5R)-3,4-dihydroxy-4-[(1S)-hydroxy-4- methylpent-3-en-1-yl]-2-(2- methylbutanoyl)-5-(3-methylbut-2-en-1- yl)cyclopent-2-en-1-one | hexahydro (6S) trans ad iso- alpha acid | 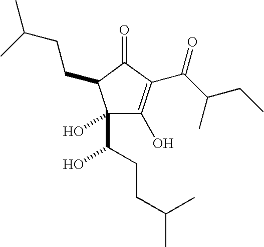 |
| (4S,5R)-3,4-dihydroxy-4-[(1R)-hydroxy-4- methylpent-3-en-1-yl]-2-(2- methylbutanoyl)-5-(3-methylbut-2-en-1- yl)cyclopent-2-en-1-one | hexahydro (6R) trans ad iso- alpha acid | 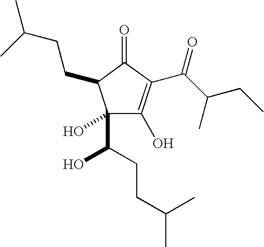 |

The term “modulate” or “modulation” is used herein to mean the up or down regulation of expression or activity of the enzyme by a compound, ingredient, etc., to which it refers.

As used herein, the term “protein kinase” represent transferase class enzymes that are able to transfer a phosphate group from a donor molecule to an amino acid residue of a protein. See Kostich, M., et. al., Human Members of the Eukaryotic Protein Kinase Family, Genome Biology 3(9):research0043.1-0043.12, 2002 herein incorporated by reference in its entirety, for a detailed discussion of protein kinases and family/group nomenclature.

Representative, non-limiting examples of kinases include Abl, Abl(T315I), ALK, ALK4, AMPK, Arg, Arg, ARK5, ASK1, Aurora-A, Axl, Blk, Bmx, BRK, BrSK1, BrSK2, BTK, CaMKI, CaMKII, CaMKIV, CDK1/cyclinB, CDK2/cyclinA, CDK2/cyclinE, CDK3/cyclinE, CDK5/p25, CDK5/p35, CDK6/cyclinD3, CDK7/cyclinH/MAT1, CDK9/cyclin T1, CHK1, CHK2, CK1(y), CK1δ, CK2, CK2α2, cKit(D816V), cKit, c-RAF, CSK, cSRC, DAPK1, DAPK2, DDR2, DMPK, DRAK1, DYRK2, EGFR, EGFR(L858R), EGFR(L861Q), EphA1, EphA2, EphA3, EphA4, EphA5, EphA7, EphA8, EphB1, EphB2, EphB3, EphB4, ErbB4, Fer, Fes, FGFR1, FGFR2, FGFR3, FGFR4, Fgr, Flt1, Flt3(D835Y), Flt3, Flt4, Fms, Fyn, GSK3β, GSK3α, Hck, HIPK1, HIPK2, HIPK3, IGF-1R, IKKβ, IKKα, IR, IRAK1, IRAK4, IRR, ITK, JAK2, JAK3, JNK1α1, JNK2α2, JNK3, KDR, Lek, LIMK1, LKB1, LOK, Lyn, Lyn, MAPK1, MAPK2, MAPK2, MAPKAP-K2, MAPKAP-K3, MARK1, MEK1, MELK, Met, MINK, MKK4, MKK6, MKK7β, MLCK, MLK1, Mnk2, MRCKβ, MRCKα, MSK1, MSK2, MSSK1, MST1, MST2, MST3, MuSK, NEK2, NEK3, NEK6, NEK7, NLK , p70S6K, PAK2, PAK3, PAK4, PAK6, PAR-1Bα, PDGFRβ, PDGFRα, PDK1, PI3K beta, PI3K delta, PI3K gamma, Pim-1, Pim-2, PKA(b), PKA, PKBβ, PKBα, PKBγ, PKCμ, PKCβI, PKCβII, PKCα, PKCγ, PKCδ, PKCε, PKCζ, PKCη, PKCθ, PKCι, PKD2, PKG1β, PKG1α, Plk3, PRAK, PRK2, PrKX, PTK5, Pyk2, Ret, RIPK2, ROCK-I, ROCK-II, ROCK-II, Ron, Ros, Rse, Rsk1, Rsk1, Rsk2, Rsk3, SAPK2a, SAPK2a(T106M), SAPK2b, SAPK3, SAPK4, SGK, SGK2, SGK3, SIK, Snk, SRPK1, SRPK2, STK33, Syk, TAK1, TBK1, Tie2, TrkA, TrkB, TSSK1, TSSK2, WNK2, WNK3, Yes, ZAP-70, ZIPK. In some embodiments, the kinases may be ALK, Aurora-A, Axl, CDK9/cyclin T1, DAPK1, DAPK2, Fer, FGFR4, GSK3β, GSK3α, Hck, JNK2α2, MSK2, p70S6K, PAK3, PI3K delta, PI3K gamma, PKA, PKBβ, PKBα, Rse, Rsk2, Syk, TrkA, and TSSK1. In yet other embodiments the kinase is selected from the group consisting of ABL, AKT, AURORA, CDK, DBF2/20, EGFR, EPH/ELK/ECK, ERK/MAPKFGFR, GSK3, IKKB, INSR, MK DOM 1/2, MARK/PRKAA, MEK/STE7, MEK/STE11, MLK, mTOR, PAK/STE20, PDGFR, PI3K, PKC, POLO, SRC, TEC/ATK, and ZAP/SYK.

The methods and compositions of the present invention are intended for use with any mammal that may experience the benefits of the methods of the invention, Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

As used herein “cancer” refers to any of various benign or malignant neoplasms characterized by the proliferation of anaplastic cells that, if malignant, tend to invade surrounding tissue and metastasize to new body sites. Representative, non-limiting examples of cancers considered within the scope of this invention include brain, breast, colon, kidney, leukemia, liver, lung, and prostate cancers. Non-limiting examples of cancer associated protein kinases considered within the scope of this invention include ABL, AKT, AMPK, Aurora, BRK, CDK, CHK, EGFR, ERB, EGFR, IGFR, KIT, MAPK, mTOR, PDGFR, PI3K, PKC, and SRC.

The term “angiogenesis” refers to the growth of new blood vessels—an important natural process occurring in the body. In many serious diseases states, the body loses control over angiogenesis, a condition sometime known as pathological angiogenesis. Angiogenesis-dependent diseases result when new blood vessels grow excessively. Examples of angiogenesis-related disorders include chronic inflammation (e.g., rheutatoid arthritis or Crohn's disease), diabetes (e.g., diabetic retinopathy), macular degeneration, psoriasis, endometriosis, and ocular disorders and cancer. “Ocular disorders” (e.g., corneal or retinal neovascularization), refers to those disturbances in the structure or function of the eye resulting from developmental abnormality, disease, injury, age or toxin. Non-limiting examples of ocular disorders considered within the scope of the present invention include retinopathy, macular degeneration or diabetic retinopathy. Ocular disorder associated kinases include, without limitation, AMPK, Aurora, EPN, ERB, ERK, FMS, IGFR, MEK, PDGFR, PI3K, PKC, SRC, and VEGFR.

Any condition or disorder that is associated with or that results from pathological angiogenesis, or that is facilitated by neovascularization (e.g., a tumor that is dependent upon neovascularization), is amenable to treatment with a substituted 1,3-cyclopentadione compound.

Conditions and disorders amenable to treatment include, but are not limited to, cancer; proliferative retinopathies such as diabetic retinopathy, age-related maculopathy, retrolental fibroplasia; excessive fibrovascular proliferation as seen with chronic arthritis; psoriasis; and vascular malformations such as hemangiomas, and the like.

The compositions and methods of the present invention are useful in the treatment of both primary and metastatic solid tumors, including carcinomas, sarcomas, leukemias, and lymphomas. Of interest is the treatment of tumors occurring at a site of angiogenesis. Thus, the methods are useful in the treatment of any neoplasm, including, but not limited to, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The instant methods are also useful for treating solid tumors arising from hematopoietic malignancies such as leukemias (i e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, the instant methods are useful for reducing metastases from the tumors described above either when used alone or in combination with radiotherapy, other chemotherapeutic and/or anti-angiogenesis agents.

As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “pharmaceutically acceptable” is used in the sense of being compatible with the other ingredients of the compositions and not deleterious to the recipient thereof,

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

It will be appreciated by those of skill in the art that the number of administrations of the compounds according to the invention will vary from patient to patient based on the particular medical status of that patient at any given time including other clinical factors such as age, weight and condition of the mammal and the route of administration chosen,

As used herein, “symptom” denotes any sensation or change in bodily function that is experienced by a patient and is associated with a particular disease, i.e., anything that accompanies “X” and is regarded as an indication of “X”'s existence. It is recognized and understood that symptoms will vary from disease to disease or condition to condition. By way of non-limiting examples, symptoms associated with autoimmune disorders include fatigue, dizziness, malaise, increase in size of an organ or tissue (for example, thyroid enlargement in Grave's Disease), or destruction of an organ or tissue resulting in decreased functioning of an organ or tissue (for example, the islet cells of the pancreas are destroyed in diabetes).

“Inflammation” or “inflammatory condition” as used herein refers to a local response to cellular injury that is marked by capillary dilatation, leukocytic infiltration, redness, heat, pain, swelling, and often loss of function and that serves as a mechanism initiating the elimination of noxious agents and of damaged tissue. Representative symptoms of inflammation or an inflammatory condition include, if confined to a joint, redness, swollen joint that's warm to touch, joint pain and stiffness, and loss of joint function. Systemic inflammatory responses can produce “flu-like” symptoms, such as, for instance, fever, chills, fatigue/loss of energy, headaches, loss of appetite, and muscle stiffness.

A first aspect of the invention discloses methods to treat a cancer responsive to protein kinase modulation in a mammal in need, where the method comprises administering to the mammal a therapeutically effective amount of a substituted 1,3-cyclopentadione compound. In some embodiments of this invention, the substituted 1,3-cyclopentadione compound is selected from the group consisting of dihydro-(Rho) isoalpha acids; tetra-hydroisoalpha acids; hexa-hydroisoalpha acids; beta acids; their individual analogs; and mixtures thereof. In other embodiments of this aspect, the substituted 1,3-cyclopentadione compound is selected from the group consisting of tetrahydro-isohumulone, tetrahydro-isocohumulone, and tetrahydro-adhumulone.

In other embodiments of this aspect, the protein kinase modulated is selected from the group consisting of Abl(T315I), Aurora-A, Bmx, BTK, CaMKI, CaMKIδ, CDK2/cyclinA, CDK3/cyclinE, CDK9/cyclin T1, CK1(y), CK1γ1, CK1γ2, CK1γ3, CK1δ, cSRC, DAPK1, DAPK2, DRAK1, EphA2, EphA8, Fer, FGFR2, FGFR3, Fgr, Flt4, PI3K, Pim-1, Pim-2, PKA, PKA(b), PKBβ, PKBα, PKBγ, PRAK, PrKX, Ron, Rsk1, Rsk2, SGK2, Syk, Tie2, TrkA, and TrkB.

In still other embodiments, the cancer responsive to kinase modulation is selected from the group consisting of bladder, breast, cervical, colon, lung, lymphoma, melanoma, prostate, thyroid, and uterine cancer. Other cancer types treatable by the methods of the present invention are described above.

A second aspect of the invention describes methods to treat angiogenic conditions responsive to protein kinase modulation in a mammal in need. The method comprises administering to the mammal a therapeutically effective amount of a substituted 1,3-cyclopentadione compound. In some embodiments of this invention, the substituted 1,3-cyclopentadione compound is selected from the group consisting of dihydro-(Rho) isoalpha acids; tetra-hydroisoalpha acids; hexa-hydroisoalpha acids; beta acids; their individual analogs; and mixtures thereof. In other embodiments of this aspect, the substituted 1,3-cyclopentadione compound is selected from the group consisting of tetrahydro-isohumulone, tetrahydro-isocohumulone, and tetrahydro-adhumulone.

In one embodiment of this aspect, the protein kinases modulated are those associated with the regulation of angiogenesis including, without limitation, ATK, MAPK, PRAK, PI3K, PKC, GSK, FGFR, BTK, PDK, SYK, MSK and IKKb,

In another embodiment of this second aspect, the method generally involves administering to a mammal a substituted 1,3-cyclopentadione compound in an amount effective to reduce angiogenesis. An effective amount for reduction of angiogenesis, in vivo, is any amount that reduces angiogenesis between at least about 5% to 100% as compared to an untreated (e.g., a placebo-treated) control.

Whether angiogenesis is reduced can be determined using any known method. Methods of determining an effect of an agent on angiogenesis are known in the art and include, but are not limited to, inhibition of neovascularization into implants impregnated with an angiogenic factor; inhibition of blood vessel growth in the cornea or anterior eye chamber; inhibition of endothelial cell proliferation, migration or tube formation in vitro; the chick chorioallantoic membrane assay; the hamster cheek pouch assay; the polyvinyl alcohol sponge disk assay. Such assays are well known in the art and have been described in numerous publications, including, e.g., Auerbach et al. (Pharmacol. Ther. 51(1):1-11(1991)), and references cited therein.

In another embodiment that relates to both first and second aspects of the present invention, the invention further provides methods for treating a condition or disorder associated with or resulting from pathological angiogenesis. In the context of cancer therapy, a reduction in angiogenesis according to the methods of the invention effects a reduction in tumor size; and a reduction in tumor metastasis. Whether a reduction in tumor size is achieved can be determined, e.g., by measuring the size of the tumor, using standard imaging techniques. Whether metastasis is reduced can be determined using any known method. Methods to assess the effect of an agent on tumor size are well known, and include imaging techniques such as computerized tomography and magnetic resonance imaging. In accordance to this embodiment, an effective amount of a substituted 1,3-cyclopentadione compound is administered to a mammal in need thereof, which causes a reduction of the tumor size, in vivo, by at least about 5% or more, when compared to an untreated (e.g., a placebo-treated) control.

A third aspect of the invention describes methods to modulate inflammation associated with cancer or angiogenesis The method comprises administering to the mammal a therapeutically effective amount of a substituted 1,3-cyclopentadione compound. In one embodiment, an effective amount of a substituted 1,3-cyclopentadione compound is administered to a mammal in need thereof, which results in reduction of inflammation or inflammation associated symptoms such as pain, by at least about 10% or more, when compared to an untreated (e.g., a placebo-treated) control. Whether a reduction in inflammation is achieved can be determined, e.g., by clinical observation or by measuring the modulation or inhibition of PGE2, nitric oxide or various DNA or protein markers of inflammation.

A fourth aspect of the invention describes compositions to treat or inhibit angiogenesis, cancers and/or their associated inflammatory pathways responsive or susceptible to protein kinase modulation, in a mammal in need thereof. The compositions comprise a therapeutically effective amount of a substituted 1,3-cyclopentadione compound; wherein the therapeutically effective amount modulates an angiogenic associated protein kinase, a cancer associated protein kinase and/or an inflammation associated protein kinase. In some embodiments of this aspect of the invention, the substituted 1,3-cyclopentadione compound is selected from the group consisting of dihydro-(Rho) isoalpha acids; tetra-hydroisoalpha acids; hexa-hydroisoalpha acids; beta acids; their individual analogs; and mixtures thereof In other embodiments of this aspect, the substituted 1,3-cyclopentadione compound is selected from the group consisting of tetrahydro-isohumulone, tetrahydro-isocohumulone, and tetrahydro-adhumulone.

Compositions used in the methods of this aspect may further comprise one or more members selected from the group consisting of antioxidants, vitamins, minerals, proteins, fats, and carbohydrates, or a pharmaceutically acceptable excipient selected from the group consisting of coatings, isotonic and absorption delaying agents, binders, adhesives, lubricants, disintergrants, coloring agents, flavoring agents, sweetening agents, absorbants, detergents, and emulsifying agents.

In other embodiment of this fourth aspect, the compositions further comprise a pharmaceutically acceptable excipient selected from the group consisting of coatings, isotonic and absorption delaying agents, binders, adhesives, lubricants, disintergrants, coloring agents, flavoring agents, sweetening agents, absorbants, detergents, and emulsifying agents.

To practice the method of the present invention, the above-described compounds and compositions can be administered orally, parenterally, by inhalation spray, topically, rectally, nasally, vaginally or via an implanted reservoir.

A composition for oral administration can be any orally acceptable dosage form including, but not limited to, capsules, tablets, powder, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

The carrier in the therapeutic composition must be ‘acceptable’ in the sense of being compatible with the active ingredient of the formulation (and preferably, capable of stabilizing it) and not deleterious to the subject to be treated. For example, solubilizing agents such as cyclodextrins, which form specific, more soluble complexes with the 1,3-cyclopentadione compounds, or one or more solubilizing agents, can be utilized as pharmaceutical excipients for delivery of the fused bicyclic heterocyclic compounds. Examples of other carriers include colloidal silicon dioxide, magnesium stearate, cellulose, sodium lauryl sulfate, and D&C Yellow #10.

The dose of a substituted 1,3-cyclopentadione compound of the invention administered to a subject, particularly a human, in the context of the present invention should be sufficient to effect a therapeutic reduction in angiogenesis, tumor size/progression or inflammation in the subject over a reasonable time frame. The dose will he determined by, among other considerations, the potency of the particular substituted 1,3-cyclopentadione compound employed and the condition of the subject, as well as the body weight of the subject to be treated.

In determining the effective amount of a substituted 1,3-cyclopentadione compound in the reduction of, for example, angiogenesis, the route of administration, the kinetics of the release system (e.g., pill, gel or other matrix), and the potency of the substituted 1,3-cyclopentadione compound are considered so as to achieve the desired anti-angiogenic effect with minimal adverse side effects. The substituted 1,3-cyclopentadione compound will typically be administered to the subject being treated for a time period ranging from a day to a few weeks, consistent with the clinical condition of the treated subject.

As will be readily apparent to the ordinarily skilled artisan, the dosage is adjusted for substituted 1,3-cyclopentadione compounds according to their potency and/or efficacy relative to a standard. See, for example, Example 17. A dose may be in the range of about 0.01 mg to 1000 mg, or about 0.1 to 100 mg, or about 0.5 to 50 mg, or about 1 to 25 mg, given 1 to 20 times daily, and can be up to a total daily dose of about 0.1 mg to 10000 mg. If applied topically, for the purpose of a systemic effect, the patch or cream is designed to provide for systemic delivery of a dose in the range of about 0.01 mg to 1000 mg, or about 0.1 to 100 mg, or about 0.5 to 50 mg, or about 1 to 25 mg. If the purpose of the topical formulation (e.g., cream) is to provide a local anti-angiogenic effect, the dose is generally in the range of about 0.001 mg to 10 mg or about 0,01 to 10 mg, or about 0.1 to 10 mg.

Regardless of the route of administration, the dose of substituted 1,3-cyclopentadione compound can be administered over any appropriate time period, e.g., over the course of 1 to 24 hours, over one to several days, etc. Furthermore, multiple doses can be administered over a selected time period. A suitable dose can be administered in suitable subdoses per day, particularly in a prophylactic regimen The precise treatment level will be dependent upon the response of the subject being treated.

In some embodiments relating to all aspects of the present invention, a substituted 1,3-cyclopentadione compound is administered alone or in a combination therapy with one or more other substituted 1,3-cyclopentadiones and/or other therapeutic agents, including an inhibitor of angiogenesis; and optionally a cancer chemotherapeutic agent.

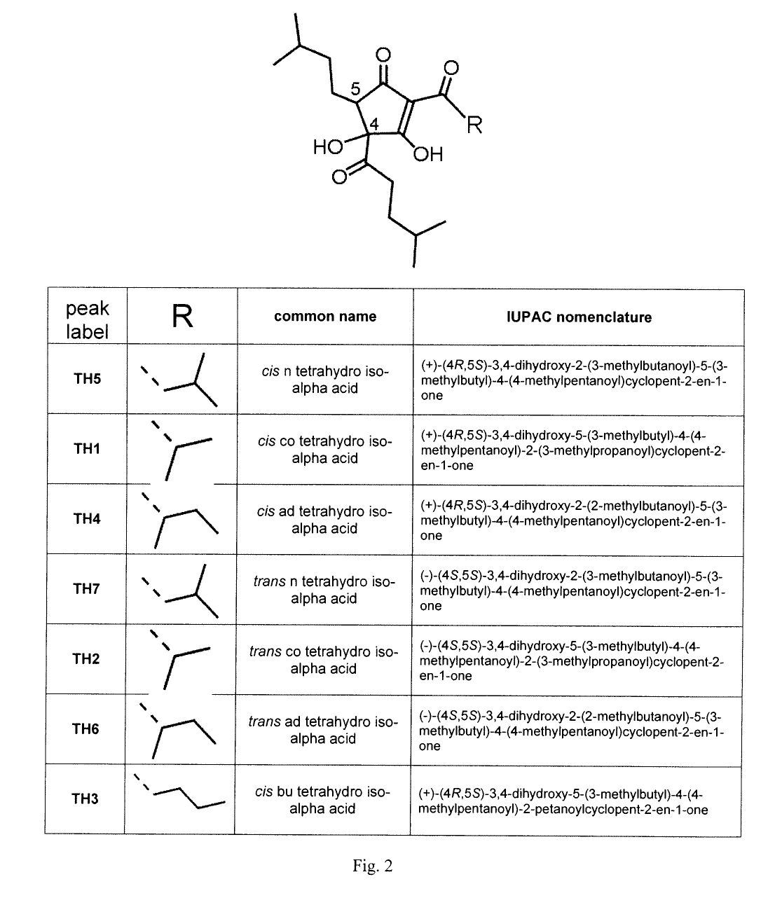

In one embodiment, an effective amount a composition containing of one or more of individual (n), (co) or (ad) analogs of a substituted 1,3-cyclopentadione compound are administered to a mammal in need thereof as the only substituted 1,3-cyclopentadione compound(s) in the composition. The (n), (co) and (ad) analogs of a substituted 1,3-cyclopentadione compound are depicted in Tables 1-3. For example, a composition may include only TH5 (an (n) analog) as the only substituted 1,3-cyclopentadione compound in the composition. Another composition may include cis-TH5 and trans-TH7 (both are (n) analogs of tetrahydro-isoalpha acid) as the only substituted 1,3-cyclopentadione compounds in the composition. Another composition may include TH1 and TH2 (both as (co) analogs of tetrahydro-isoalpha acid) as the only substituted 1,3-cyclopentadione compounds in the composition. Another composition may include TH4 and TH6 (both as (ad) analogs of tetrahydro-isoalpha acid) as the only substituted 1,3-cyclopentadione compounds in the composition. FIG. 2 depicts the chemical structures of TH compounds.

In another embodiment, an effective amount a composition containing one or more (n) analogs of a substituted 1,3-cyclopentadione compound is administered in combination with one or more (ad) analogs of a substituted 1,3-cyclopentadione compound in accordance with the methods of the invention. For example, a composition may include TH4 (an (ad) analog) and TH5 (an (n) analog). It has been shown that TH4 and TH5 at 100 μg/mL almost completely inhibit BMX kinase. Other compositions may contain TH5 and TH6; TH7 and TH4; and TH7 and TH6.

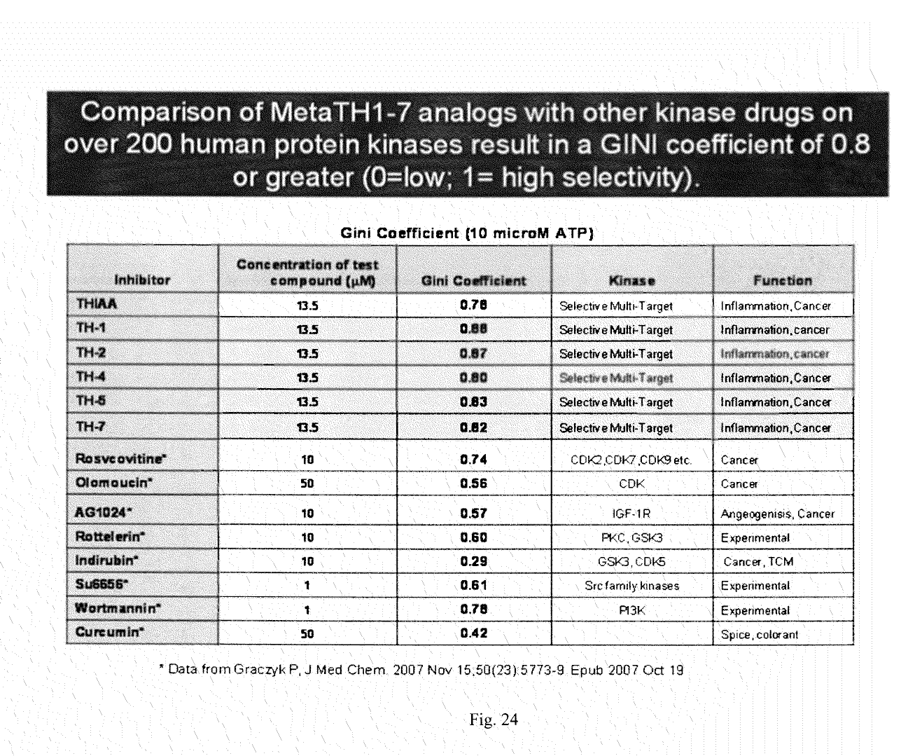

The advantage of using one or more analogs of a substituted 1,3-cyclopentadione compound in a composition is that higher doses of specific analogs can be used without toxic side effects of using those with less activity on a given target. Another advantage is achieving selectivity or specificity. For example, the tetrahydro-isocohumulone (i.e., TH1) is less preferred in both animal and in viro inflammation models. However, TH1 and TH2 are more specific and are preferred in the treatment of certain cancers due to having a higher Gini coefficient (see FIGS. 23-24). Gini coefficient is a measure of selectivity of kinase inhibitors against a panel of kinases (Craczyk P., J Med Chem. November 15:50(23)5773-9 (2007)). Briefly, nonselective inhibitors are characterized by Gini coeffients close to zero while highly selective compounds exhibit Gini coefficients close to 1. It has further been observed that while TH4 and TH5 are more active at 100 μg/ml in inhibiting BMX (inhibit it almost completely), TH1, TH2 have about 50% of the activity in comparison. This same type of selectivity is observed for TRKB, PrKX, CK1 delta, BTK, JAK3, RSK1, CDK2/cyclinE, EGFR(L858R), NEK, PKB beta, Arg, Src(1-530), TrkA, Rsk4. Further, as shown in FIG. 23, although TH7's Gini coefficient profile is in the middle, TH7 has been observed to act more similar to TH4 and TH5 over the dose range. The Gini coefficients of TH1-7 have also been compared with the Gini coefficients of compounds known to function as anti-cancer or anti-angiogeneis drugs (FIG. 24). The data also indicate that TH1-7 are individually more selective protein kinase inhibitors than THIAA which is a misture of same. Another advantage of using a composition of two or more analogs of a substituted 1,3-cyclopentadione compound can be modulation of more kinase targets than when only a single analog is used.

Accordingly, in some embodiments relating to all aspects of the present invention the following exemplary combinations of analogs of a substituted 1,3-cyclopentadione compound are contemplated, which are expected to have the benefits specified in the parentheses that follows each combination: (i) tetrahydro-isohumulone cis and trans: TH5+TH7 (benefit: more targets); (ii) tetrahydro-isoadhumulone cis and trans: TH4+TH6 (benefit: more targets); (iii) (n) and (ad) families: TH5+TH4; TH5+TH6; TH7+TH4; TH7+TH6 (benefit: more targets); (iv) tetrahydroiso-cohumulone cis and trans: TH1+TH2 (benefit: higher gini); and (v) (n) and (co) families TH1+TH5; TH2+TH5; TH1+TH7; TH2+TH7 (benefit: more targets).

With regard to other combination therapies, a substituted 1,3-cyclopentadione compound of the invention can be used in combination with suitable chemotherapeutic agents including, but are not limited to, the alkylating agents, e.g. Cisplatin, Cyclophosphamide, Altretamine; the DNA strand-breakage agents, such as Bleomycin; DNA topoisomerase II inhibitors, including intercalators, such as Amsacrine, Dactinomycin, Daunorubicin, Doxorubicin, Idarubicin, and Mitoxantrone; the nonintercalating topoisomerase II inhibitors such as, Etoposide and Teniposide; the DNA minor groove binder Plicamycin; alkylating agents, including nitrogen mustards such as Chlorambucil, Cyclophosphamide, Isofamide, Mechlorethamine, Melphalan, Uracil mustard; aziridines such as Thiotepa; methanesulfonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine; antimetabolites, including folate antagonists such as Methotrexate and trimetrexate; pyrimidine antagonists, such as Fluorouracil, Fluorodeoxyuridine, CB3717, Azacytidine, Cytarabine; Floxuridine purine antagonists including Mercaptopurine, 6-Thioguanine, Fludarabine, Pentostatin; sugar modified analogs include Cyctrabine, Fludarabine; ribonucleotide reductase inhibitors including hydroxyurea; Tubulin interactive agents including Vincristine Vinblastine, and Paclitaxel; adrenal corticosteroids such as Prednisone, Dexamethasone, Methylprednisolone, and Prodnisolone; hormonal blocking agents including estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone; and the like.

The substituted 1,3-cyclopentadione compound may be administered with other anti-angiogenic agents. Furthermore, a substituted 1,3-cyclopentadione compound of the invention can be used in combination with anti-angiogenic agents including, but are not limited to, angiostatic steroids such as heparin derivatives and glucocorticosteroids; thrombospondin; cytokines such as IL-12; fumagillin and synthetic derivatives thereof, such as AGM 12470; interferon-α; endostatin; soluble growth factor receptors; neutralizing monoclonal antibodies directed against growth factors; and the like.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### Examples

### Example 1

### Effects of Meta-THc on Protein Kinases

As stated above, kinases represent transferase class enzymes that transfer a phosphate group from a donor molecule (usually ATP) to an amino acid residue of a protein (usually threonine, serine or tyrosine). Kinases are used in signal transduction for the regulation of enzymes, i.e., they can inhibit or activate an enzyme, such as an enzyme involved in cholesterol biosynthesis, amino acid transformation, or glycogen turnover. While most kinases are specialized to a single kind of amino acid residue for phosphorylation, some kinases exhibit dual activity in that they can phosphorylate two different kinds of amino acids.

Methods—The dose responsiveness for kinase inhibition (reported as a percent of control) of a Meta-THc preparation was tested at approximately 1, 10, 25, and 50 ug/ml on 86 selected kinases as presented in Table 1 below. The inhibitory effect of the present method on human kinase activity was tested in the KinaseProfiler™ Assay (Upstate Cell Signaling Solutions, Upstate USA, Inc., Charlottesville, Va., USA). The assay protocols for specific kinases are summarized at www.upstate.com/img/pdf/kp_protocols_full.pdf, incorporated herein by reference thereto.

Results—Meta-THc displayed a dose dependent inhibition of kinase activity for many of the kinases examined with inhibition of FGFR2 of 7%, 16%, 77%, and 91% at 1, 5, 25, and 50 μg/ml, respectively. Similar results were observed for FGFR3 (0%, 6%, 61%, and 84%) and TrkA (24%, 45%, 93%, and 94%) at 1, 5, 25, and 50 μg/ml respectively.

The inhibitory effects of Meta-THc on the kinases tested are shown in Table 4 below.

TABLE 4
Dose response effect (as % of Control) of
Meta-THc on selected protein kinases

| Kinase | 1 ug/ml | 5 ug/ml | 25 ug/ml | 50 ug/ml |
|---|---|---|---|---|
| Abl(T315I) | 104 | 95 | 68 | 10 |
| ALK4 | 127 | 112 | 108 | |
| AMPK | 135 | 136 | 139 | 62 |
| Aurora-A | 102 | 86 | 50 | 5 |
| Bmx | 110 | 105 | 57 | 30 |
| BTK | 104 | 86 | 58 | 48 |
| CaMKI | 163 | 132 | 65 | 16 |
| CaMKIIβ | 106 | 102 | 90 | 71 |
| CaMKIIγ | 99 | 101 | 87 | 81 |
| CaMKIIδ | 99 | 103 | 80 | 76 |
| CaMKIV | 99 | 117 | 120 | 126 |
| CaMKIδ | 91 | 95 | 61 | 43 |
| CDK1/cyclinB | 82 | 101 | 77 | 66 |
| CDK2/cyclinA | 118 | 113 | 87 | 50 |
| CDK2/cyclinE | 87 | 79 | 73 | 57 |
| CDK3/cyclinE | 113 | 111 | 105 | 32 |
| CDK5/p25 | 102 | 100 | 85 | 54 |
| CDK5/p35 | 109 | 106 | 89 | 80 |
| CDK6/cyclinD3 | 114 | 113 | 112 | 70 |
| CDK9/cyclin T1 | 106 | 93 | 66 | 36 |
| CHK1 | 116 | 118 | 149 | 148 |
| CHK2 | 111 | 116 | 98 | 68 |
| CK1(y) | 101 | 101 | 55 | |
| CK1γ1 | 101 | 100 | 42 | 43 |
| CK1γ2 | 94 | 85 | 33 | 48 |
| CK1γ3 | 99 | 91 | 23 | 18 |
| CK1δ | 109 | 97 | 65 | 42 |
| cKit(D816H) | 113 | 113 | 69 | 75 |
| CSK | 110 | 113 | 92 | 137 |
| cSRC | 105 | 103 | 91 | 17 |
| DAPK1 | 62 | 34 | 21 | 14 |
| DAPK2 | 60 | 54 | 41 | 17 |
| DRAK1 | 113 | 116 | 75 | 18 |
| EphA2 | 110 | 112 | 85 | 31 |
| EphA8 | 110 | 110 | 83 | 43 |
| EphB1 | 153 | 177 | 196 | 53 |
| ErbB4 | 124 | 125 | 75 | 56 |
| Fer | 85 | 41 | 24 | 12 |
| Fes | 112 | 134 | 116 | 57 |
| FGFR1 | 109 | 110 | 110 | 111 |
| FGFR1(V561M) | 97 | 106 | 91 | 92 |
| FGFR2 | 126 | 115 | 58 | 7 |
| FGFR3 | 112 | 94 | 39 | 16 |
| FGFR4 | 122 | 93 | 83 | 58 |
| Fgr | 121 | 120 | 110 | 47 |
| Flt4 | 126 | 119 | 85 | 31 |
| IKKα | 139 | 140 | 140 | 102 |
| JNK1α1 | 71 | 118 | 118 | 107 |
| JNK2α2 | 94 | 97 | 98 | 101 |
| JNK3 | 121 | 78 | 58 | 44 |
| KDR | 106 | 107 | 104 | 126 |
| Lck | 97 | 105 | 125 | 88 |
| LKB1 | 145 | 144 | 140 | 140 |
| MAPK2 | 99 | 109 | 112 | 102 |
| Pim-1 | 103 | 100 | 44 | 44 |
| Pim-2 | 103 | 109 | 83 | 22 |
| PKA(b) | 104 | 77 | 32 | 0 |
| PKA | 104 | 101 | 90 | 25 |
| PKBβ | 117 | 102 | 27 | 33 |
| PKBα | 103 | 101 | 49 | 50 |
| PKBγ | 107 | 109 | 99 | 33 |
| PKCμ | 90 | 90 | 93 | 87 |
| PKCβII | 99 | 107 | 103 | 64 |
| PKCα | 110 | 111 | 112 | 102 |
| PKCγ | 86 | 95 | 77 | 62 |
| PKCδ | 97 | 93 | 84 | 87 |
| PKCε | 76 | 88 | 88 | 90 |
| PKCζ | 93 | 100 | 107 | 103 |
| PKCη | 82 | 99 | 103 | 90 |
| PKCθ | 93 | 95 | 86 | 90 |
| PKCι | 77 | 90 | 93 | 134 |
| PRAK | 99 | 81 | 21 | 33 |
| PrKX | 92 | 76 | 32 | 38 |
| Ron | 120 | 110 | 97 | 42 |
| Ros | 105 | 105 | 94 | 93 |
| Rsk1 | 101 | 87 | 48 | 31 |
| Rsk2 | 100 | 85 | 40 | 14 |
| SGK | 98 | 103 | 79 | 77 |
| SGK2 | 117 | 110 | 45 | 18 |
| Syk | 99 | 93 | 55 | 17 |
| TBK1 | 101 | 100 | 82 | 56 |
| Tie2 | 109 | 115 | 100 | 32 |
| TrkA | 107 | 65 | 30 | 15 |
| TrkB | 97 | 96 | 72 | 21 |
| TSSK2 | 112 | 111 | 87 | 66 |
| ZIPK | 106 | 101 | 74 | 59 |

### Example 2

### Isolation and Identification of Meta-THc Components

High speed counter current separation was conducted to isolated and identify the components of a Meta-THc sample. A modified hops extract containing tetrahydro iso-alpha acids was obtained from Hopsteiner (Yakima, Wash.) as a pure solid. This material was partitioned between dilute H₂SO₄ (aq) pH=2.0 and hexanes and extracted several times with hexanes. The hexanes were collected, dried (NaSO₄) and filtered to remove the NaSO₄ and concentrated in vacuo to yield a waxy solid.

High Speed Counter Current (HSCCC) apparatus—Separations were performed on a Pharma-Tech Research Corporation CCC-1000 model counter-current chromatograph with semi-preparative centrifuge coils (total volume 325 mL) installed. Samples were injected into the system using a Rheodyne manual injector with a 10.0 mL sample loop. A Shimadzu LC-20AT Pump (switchable between four solvents) was used in conjunction with a Shimadzu CBM-20A system controller. Flow from the Pharma-Tech CCC-1000 went through a Shimadzu SPD-10AVvp UV-VIS detector (monitoring at 254 nm) and to a Shimadzu FRC-10A fraction collector with a large-scale fractionation kit installed (allowing fraction volumes up to 1,000 mL).

The CCC-1000 was operated in head-to-tail configuration and descending mode. The upper, stationary phase (methyl acetate) was pumped at a flow rate of 1.0 mL/min through the lower, stationary phase (0.1 M triethanolamine-pH 7.4) while rotation of the coils was held constant at 680 RPM. The sample was dissolved in 10.0 mL of lower, stationary phase and injected directly into the system.

Preparation of two-phase solvent system—The 0.1 M triethanolamine-pH 7.4 buffer was prepared by dissolving 13.25 mL of triethanolamine in 1.0 L of deionized water. The pH was adjusted to 7.4 with dilute hydrochloric acid. The aqueous buffer was thoroughly mixed with methyl acetate by repeated mixing and settling using a large separatory funnel, and a small amount of lower phase added to the upper phase and vice versa to ensure the solutions were saturated.

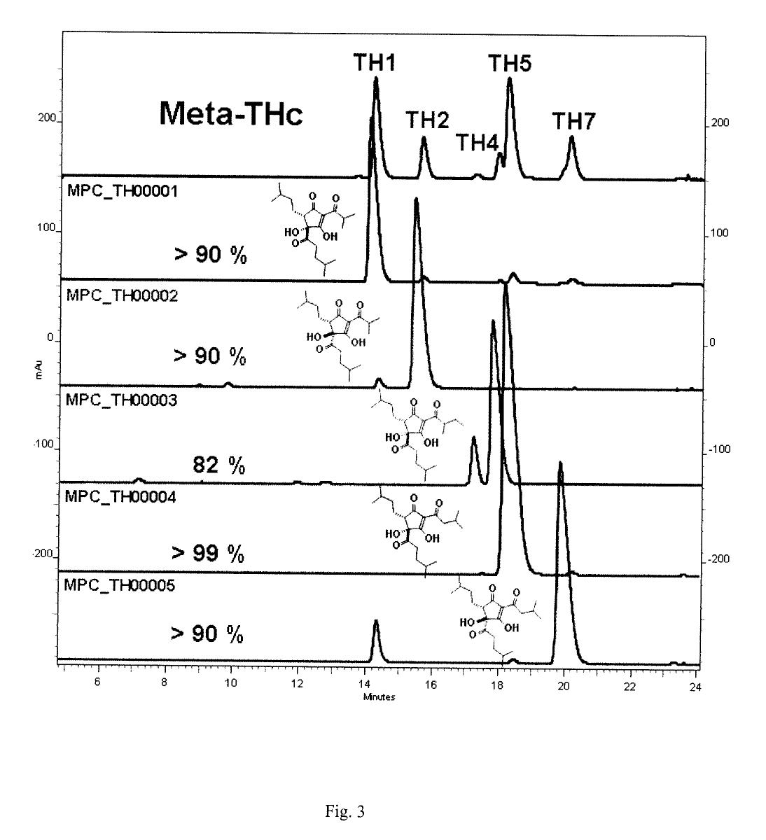

Results—FIG. 3 depicts a a representative chromatogram of a Meta-THc composition. The top panel identifies the chromatagraphic peaks comprising the Meta-THc components of the mixture whereas the subsequent panels describe the chromatagraphic profile of the isolation fractions comprising the peaks.

The percent homogeneity of each fraction, the amount isolated in each fraction and the percent recovery based upon the initial amount of material submitted to HSCCC purification are presented in Table 5 below.

TABLE 5
Purity of fractions isolated by HSCCC
Purity of CCC fractions based on peak area (HPLC, 254 nm)

| | | | | | | | | | | | | | | Vial |
|---|---|---|---|---|---|---|---|---|---|---|---|---|---|---|
| | Vial 32 | Vial 33 | Vial 34 | Vial 35 | Vial 36 | Vial 37 | Vial 38 | Vial 39 | Vial 40 | Vial 41 | Vial 42 | Vial 43 | Vial 44 | 45 |
| TH1 | | 79.5 | 82.8 | 77.5 | 57.1 | 38.4 | 11.9 | 0.9 | | | | | | |
| TH2 | 81 | 9.9 | | | | | | | | | | | | |
| TH3 | | | | | | | | | | | | 0.7 | 7.4 | 6.3 |
| TH4 | | | | | | | | | | | | 6.2 | 91.3 | 92.2 |
| TH5 | | | | 3.9 | 28.5 | 52.5 | 84.3 | 97.6 | 98.9 | 99 | 99.1 | 92.4 | | |
| TH6 | | 6.6 | 16.3 | 18.6 | 14.5 | 8.7 | 3.8 | 1.5 | 1.1 | 1 | 0.9 | 0.6 | | |

### Example 3

### Effects of Meta-THc on Protein Kinases

Methods—The dose responsiveness for kinase inhibition (reported as a percent of control) of a Meta-THc preparation and the individual components was tested at approximately 1, 5, 25, 50, and 100 ug/ml on 190 selected kinases as presented in Table 1 below. The inhibitory effect of the present invention on human kinase activity was tested in the KinaseProfiler™ Assay (Upstate Cell Signaling Solutions, Upstate USA, Inc., Charlottesville, Va., USA). The assay protocols for specific kinases are summarized at http://www.upstate.com/img/pdf/kp_protocols_full.pdf (last visited on Jun. 12, 2006).

Results—The inhibitory effects of Meta-THc on the kinases tested are shown in Tables 6-11 below.

TABLE 6

| | THI + 2 + 4 + 5 + 7 Composite (Meta-THc) | | | | |
|---|---|---|---|---|---|
| | | | | OG- | OG- |
| | OG- | OG- | OG- | 3116 @ | 3116 @ |
| | 3116 @ | 3116 @ | 3116 @ | 50 | 100 |
| | 1 μg/ml | 5 μg/ml | 25 μg/ml | μg/ml | μg/ml |
| Abl(H396P)(h) | 91 | 88 | 73 | 55 | 50 |
| Abl(M351T)(h) | 100 | 87 | 62 | 50 | 38 |
| Abl(Q252H)(h) | 89 | 86 | 58 | 45 | 44 |
| Abl(h) | 98 | 85 | 65 | 41 | 49 |
| Abl(m) | 99 | 87 | 60 | 47 | 43 |
| Abl(T315I)(h) | 100 | 91 | 79 | 65 | 52 |
| Abl(Y253F)(h) | 93 | 90 | 75 | 54 | 51 |
| ACK1(h) | 122 | 112 | 97 | 102 | 82 |
| ALK(h) | 76 | 38 | 17 | 16 | 26 |
| ALK4(h) | 96 | 95 | 85 | 68 | 48 |
| Arg(h) | 94 | 91 | 68 | 52 | 42 |
| Arg(m) | 100 | 99 | 94 | 73 | 50 |
| ARK5(h) | 100 | 97 | 82 | 64 | 75 |
| Aurora-A(h) | 92 | 79 | 40 | 41 | 27 |
| Axl(h) | 97 | 99 | 83 | 77 | 60 |
| Blk(m) | 95 | 102 | 71 | 49 | 54 |
| Bmx(h) | 91 | 94 | 84 | 77 | 43 |
| BrSK1(h) | 95 | 90 | 71 | 47 | 51 |
| BrSK2(h) | 91 | 82 | 77 | 70 | 63 |
| BTK(h) | 99 | 97 | 64 | 44 | 28 |
| CaMKI(h) | 95 | 84 | 46 | 28 | 48 |
| CaMKII(r) | 97 | 106 | 89 | 69 | 63 |
| CaMKIIβ(h) | 94 | 99 | 85 | 52 | 34 |
| CaMKIIγ(h) | 107 | 103 | 94 | 92 | 134 |
| CaMKIIδ(h) | 103 | 97 | 84 | 83 | 84 |
| CaMKIV(h) | 107 | 108 | 95 | 75 | 58 |
| CaMKIδ(h) | 91 | 93 | 92 | 75 | 80 |
| CDK1/cyclinB(h) | 99 | 101 | 91 | 71 | 58 |
| CDK2/cyclinA(h) | 105 | 106 | 92 | 83 | 63 |
| CDK2/cyclinE(h) | 99 | 103 | 75 | 60 | 42 |
| CDK3/cyclinE(h) | 108 | 100 | 96 | 79 | 45 |
| CDK5/p25(h) | 102 | 89 | 84 | 77 | 72 |
| CDK5/p35(h) | 95 | 84 | 82 | 67 | 68 |
| CDK6/cydinD3(h) | 109 | 109 | 99 | 22 | 87 |
| CDK9/cyclin T1(h) | 96 | 98 | 78 | 67 | 64 |
| CHK2(h) | 86 | 95 | 92 | 95 | 86 |
| CHK2(I157T)(h) | 100 | 92 | 91 | 73 | 53 |
| CHK2(R145W)(h) | 100 | 96 | 93 | 89 | 69 |
| CK1(y) | 101 | 102 | 102 | 82 | 73 |
| CK1γ1(h) | 93 | 89 | 82 | 50 | 47 |
| CK1γ2(h) | 103 | 96 | 64 | 52 | 32 |
| CK1γ3(h) | 96 | 92 | 53 | 29 | 27 |
| CK1δ(h) | 99 | 90 | 71 | 55 | 17 |
| cKit(D816H)(h) | 101 | 105 | 97 | 72 | 88 |
| cKit(D816V)(h) | 96 | 96 | 89 | 77 | 74 |
| cKit(h) | 84 | 86 | 64 | 71 | 76 |
| cKit(V560G)(h) | 97 | 104 | 87 | 82 | 78 |
| cKit(V654A)(h) | 100 | 96 | 84 | 81 | 81 |
| CLK2(h) | 90 | 95 | 99 | 88 | 98 |
| cSRC(h) | 101 | 104 | 105 | 112 | 92 |
| DAPK1(h) | 69 | 39 | 26 | 19 | 22 |
| DAPK2(h) | 69 | 54 | 53 | 42 | 46 |
| DCAMKL2(h) | 100 | 91 | 93 | 89 | 98 |
| DRAK1(h) | 96 | 103 | 89 | 70 | 78 |
| EGFR(L858R)(h) | 108 | 114 | 102 | 91 | 63 |
| EGFR(L861Q)(h) | 93 | 94 | 81 | 71 | 66 |
| EGFR(T790M)(h) | 100 | 95 | 97 | 97 | 99 |
| EGFR(T790M, | 98 | 99 | 90 | 72 | 81 |
| L858R)(h) | | | | | |
| EphA1(h) | 105 | 100 | 102 | 84 | 80 |
| EphA2(h) | 105 | 105 | 99 | 93 | 66 |
| EphA3(h) | 95 | 89 | 77 | 65 | 74 |
| EphA8(h) | 103 | 106 | 95 | 82 | 89 |
| EphB1(h) | 111 | 126 | 197 | 118 | 78 |
| EphB3(h) | 92 | 78 | 49 | 47 | 54 |
| EphB4(h) | 99 | 102 | 87 | 96 | 103 |
| Fer(h) | 74 | 85 | 88 | 94 | 94 |
| Fes(h) | 146 | 127 | 111 | 74 | 56 |
| FGFR1(V561M)(h) | 94 | 102 | 106 | 106 | 99 |
| FGFR2(h) | 91 | 87 | 79 | 53 | 66 |
| FGFR2(N549H)(h) | 98 | 102 | 101 | 88 | 82 |
| FGFR3(h) | 99 | 91 | 63 | 49 | 58 |
| FGFR4(h) | 93 | 70 | 40 | 37 | 41 |
| Fgr(h) | 99 | 93 | 92 | 94 | 97 |
| Flt1(h) | 96 | 97 | 94 | 88 | 85 |
| Flt3(D835Y)(h) | 96 | 104 | 101 | 95 | 92 |
| Flt3(h) | 108 | 103 | 91 | 79 | 59 |
| Flt4(h) | 103 | 112 | 90 | 69 | 52 |
| Fms(h) | 105 | 107 | 109 | 89 | 100 |
| Fyn(h) | 96 | 95 | 96 | 95 | 63 |
| GRK7(h) | 100 | 104 | 104 | 92 | 104 |
| GSK3α(h) | 93 | 84 | 53 | 36 | 37 |
| GSK3β(h) | 95 | 86 | 39 | 24 | 39 |
| Haspin(h) | 100 | 93 | 96 | 63 | 48 |
| Hck(h) | 96 | 83 | 61 | 49 | 47 |
| HIPK2(h) | 104 | 107 | 107 | 101 | 103 |
| IKKα(h) | 106 | 121 | 112 | 110 | 100 |
| IKKβ(h) | 113 | 106 | 99 | 78 | 70 |
| IR(h) | 81 | 88 | 53 | 59 | 59 |
| IRAK1(h) | 102 | 106 | 109 | 122 | 128 |
| Itk(h) | 99 | 102 | 96 | 101 | 86 |
| JAK2(h) | 98 | 105 | 98 | 94 | 85 |
| JAK3(h) | 91 | 77 | 73 | 64 | 47 |
| JNK3(h) | 98 | 98 | 90 | 78 | 78 |
| Lck(h) | 100 | 98 | 94 | 98 | 101 |
| Lyn(h) | 120 | 129 | 125 | 86 | 70 |
| Lyn(m) | 140 | 120 | 98 | 70 | 70 |
| MAPK1(h), ERK1 | 82 | 80 | 61 | 53 | 52 |
| MAPKAP-K2(h) | 99 | 107 | 83 | 48 | 52 |
| MAPKAP-K3(h) | 94 | 73 | 96 | 93 | 86 |
| MARK1(h) | 95 | 105 | 97 | 67 | 64 |
| MELK(h) | 102 | 99 | 96 | 92 | 95 |
| Met(h) | 109 | 119 | 88 | 52 | 68 |
| MKK4(m) | 96 | 115 | 88 | 87 | 101 |
| MKK7β(h) | 96 | 95 | 92 | 86 | 14 |
| MLCK(h) | 100 | 88 | 94 | 102 | 92 |
| MRCKα(h) | 97 | 100 | 100 | 91 | 90 |
| MRCKβ(h) | 102 | 106 | 102 | 100 | 75 |
| MSK1(h) | 99 | 102 | 78 | 51 | 46 |
| MSK2(h) | 99 | 84 | 63 | 33 | 31 |
| MSSK1(h) | 63 | 71 | 38 | 27 | 49 |
| MST3(h) | 117 | 117 | 71 | 25 | 28 |
| MuSK(h) | 105 | 106 | 96 | 92 | 94 |
| NEK11(h) | 93 | 92 | 90 | 65 | 43 |
| NEK2(h) | 98 | 103 | 113 | 115 | 82 |
| NEK3(h) | 99 | 98 | 94 | 105 | 84 |
| NEK6(h) | 95 | 99 | 90 | 72 | 32 |
| NEK7(h) | 98 | 99 | 89 | 86 | 67 |
| NLK(h) | 98 | 102 | 90 | 89 | 92 |
| P70S6K(h) | 98 | 98 | 100 | 69 | 70 |
| PAK2(h) | 106 | 108 | 106 | 104 | 85 |
| PAK3(h) | 112 | 85 | 51 | 38 | 42 |
| PAK4(h) | 111 | 105 | 99 | 75 | 90 |
| PAK5(h) | 94 | 102 | 96 | 77 | 62 |
| PAK6(h) | 95 | 92 | 92 | 84 | 18 |
| PAR-1Bα(h) | 99 | 111 | 101 | 81 | 89 |
| PASK(h) | 92 | 105 | 110 | 111 | 108 |
| PDGFRα(D842V)(h) | 100 | 103 | 104 | 97 | 101 |
| PDGFRα(V561D)(h) | 106 | 110 | 115 | 99 | 92 |
| PDGFRβ(h) | 93 | 91 | 76 | 66 | 49 |
| PDK1(h) | 94 | 86 | 64 | 51 | 52 |
| PhKγ2(h) | 112 | 92 | 95 | 49 | 41 |
| PI 3-Kinaseβ(h) | 94 | 95 | 89 | 54 | 49 |
| PI 3-Kinaseδ(h) | 95 | 84 | 33 | 15 | 35 |
| PI 3-Kinaseδ(h) | 100 | 91 | 31 | 5 | −3 |
| Pim-1(h) | 108 | 103 | 92 | 65 | 45 |
| Pim-2(h) | 98 | 103 | 96 | 88 | 88 |
| Pim-3(h) | 104 | 99 | 96 | 102 | 108 |
| PKA(h) | 119 | 120 | 116 | 102 | 85 |
| PKBα(h) | 97 | 103 | 102 | 97 | 116 |
| PKBβ(h) | 102 | 98 | 56 | 34 | 28 |
| PKBγ(h) | 97 | 100 | 97 | 84 | 90 |
| PKCα(h) | 97 | 101 | 91 | 81 | 75 |
| PKCβI(h) | 88 | 98 | 92 | 93 | 72 |
| PKCβII(h) | 98 | 99 | 91 | 88 | 83 |
| PKCγ(h) | 100 | 102 | 89 | 86 | 70 |
| PKCδ(h) | 86 | 104 | 83 | 75 | 99 |
| PKCε(h) | 98 | 98 | 92 | 87 | 97 |
| PKCθ(h) | 95 | 100 | 116 | 92 | 100 |
| PKG1α(h) | 98 | 97 | 100 | 93 | 71 |
| Plk3(h) | 91 | 94 | 86 | 79 | 83 |
| PRAK(h) | 68 | 36 | 17 | 12 | 18 |
| PrKX(h) | 98 | 97 | 90 | 75 | 59 |
| PTK5(h) | 102 | 102 | 104 | 102 | 110 |
| Ret(V804L)(h) | 106 | 94 | 81 | 61 | 52 |
| Ret(h) | 111 | 99 | 98 | 84 | 81 |
| Ret(V804M)(h) | 103 | 98 | 90 | 90 | 84 |
| ROCK-I(h) | 107 | 96 | 89 | 74 | 83 |
| Ron(h) | 119 | 101 | 108 | 98 | 89 |
| Rsk1(h) | 96 | 97 | 70 | 27 | 24 |
| Rsk1(r) | 97 | 97 | 80 | 54 | 21 |
| Rsk2(h) | 97 | 95 | 51 | 34 | 27 |
| Rsk3(h) | 100 | 98 | 82 | 78 | 75 |
| Rsk4(h) | 94 | 81 | 46 | 28 | 20 |
| SAPK2b(h) | 108 | 103 | 103 | 102 | 116 |
| SAPK3(h) | 98 | 105 | 104 | 113 | 113 |
| SAPK4(h) | 101 | 105 | 110 | 111 | 108 |
| SGK(h) | 97 | 101 | 100 | 81 | 60 |
| SGK2(h) | 92 | 107 | 88 | 73 | 62 |
| SIK(h) | 97 | 97 | 78 | 60 | 41 |
| Src(1-530)(h) | 105 | 101 | 101 | 90 | 37 |
| SRPK1(h) | 90 | 83 | 19 | 43 | 33 |
| SRPK2(h) | 105 | 101 | 88 | 98 | 85 |
| Syk(h) | 120 | 127 | 87 | 54 | 39 |
| TBK1(h) | 98 | 99 | 94 | 100 | 69 |
| Tie2(h) | 99 | 91 | 73 | 53 | 62 |
| Tie2(R849W)(h) | 88 | 42 | 40 | 51 | 55 |
| Tie2(Y897S)(h) | 75 | 44 | 34 | 24 | 26 |
| TLK2(h) | 94 | 98 | 91 | 85 | 107 |
| TrkA(h) | 103 | 98 | 42 | 8 | 23 |
| TrkB(h) | 107 | 137 | 135 | 111 | 55 |
| TSSK1(h) | 96 | 97 | 92 | 87 | 78 |
| TSSK2(h) | 100 | 99 | 92 | 91 | 85 |
| Txk(h) | 99 | 105 | 114 | 122 | 112 |
| ULK2(h) | 105 | 116 | 92 | 44 | 81 |
| WNK3(h) | 98 | 104 | 108 | 110 | 103 |
| Yes(h) | 96 | 88 | 84 | 87 | 102 |
| ZAP-70(h) | 105 | 103 | 99 | 96 | 101 |
| ZIPK(h) | 103 | 91 | 79 | 65 | 78 |

TABLE 7

| | TH-1 | | | | |
|---|---|---|---|---|---|
| | | | | OG- | OG- |
| | OG- | OG- | OG- | 3306 @ | 3306 @ |
| | 3306 @ | 3306 @ | 3306 @ | 50 | 100 |
| | 1 μg/ml | 5 μg/ml | 25 μg/ml | μg/ml | μg/ml |
| Abl(H396P)(h) | 100 | 90 | 89 | 79 | 47 |
| Abl(M351T)(h) | 96 | 95 | 89 | 79 | 45 |
| Abl(Q252H)(h) | 101 | 93 | 82 | 72 | 36 |
| Abl(h) | 96 | 88 | 78 | 60 | 35 |
| Abl(m) | 48 | 99 | 77 | 65 | 50 |
| Abl(T315I)(h) | 99 | 95 | 93 | 89 | 65 |
| Abl(Y253F)(h) | 101 | 102 | 94 | 81 | 47 |
| ACK1(h) | 112 | 101 | 106 | 100 | 92 |
| ALK(h) | 78 | 49 | 29 | 20 | 18 |
| ALK4(h) | 110 | 91 | 102 | 93 | 65 |
| Arg(h) | 77 | 88 | 88 | 64 | 45 |
| Arg(m) | 106 | 105 | 108 | 107 | 52 |
| ARK5(h) | 110 | 105 | 97 | 88 | 74 |
| Aurora-A(h) | 110 | 102 | 85 | 52 | 48 |
| Axl(h) | 107 | 108 | 103 | 91 | 68 |
| Blk(m) | 121 | 103 | 100 | 81 | 60 |
| Bmx(h) | 93 | 93 | 92 | 83 | 49 |
| BrSK1(h) | 100 | 95 | 96 | 79 | 53 |
| BrSK2(h) | 97 | 92 | 93 | 78 | 61 |
| BTK(h) | 99 | 99 | 79 | 68 | 34 |
| CaMKI(h) | 90 | 94 | 74 | 47 | 37 |
| CaMKII(r) | 113 | 110 | 114 | 104 | 73 |
| CaMKIIβ(h) | 107 | 105 | 100 | 83 | 55 |
| CaMKIIγ(h) | 103 | 109 | 101 | 109 | 106 |
| CaMKIIδ(h) | 106 | 96 | 90 | 98 | 86 |
| CaMKIV(h) | 104 | 113 | 114 | 102 | 49 |
| CaMKIδ(h) | 93 | 90 | 96 | 91 | 80 |
| CDK1/cyclinB(h) | 103 | 102 | 99 | 94 | 74 |
| CDK2/cyclinA(h) | 114 | 112 | 108 | 97 | 83 |
| CDK2/cyclinE(h) | 93 | 103 | 91 | 78 | 52 |
| CDK3/cyclinE(h) | 112 | 116 | 103 | 112 | 59 |
| CDK5/p25(h) | 105 | 95 | 98 | 95 | 69 |
| CDK5/p35(h) | 105 | 110 | 97 | 91 | 68 |
| CDK6/cyclinD3(h) | 115 | 110 | 99 | 106 | 96 |
| CDK9/cyclin T1(h) | 95 | 97 | 97 | 86 | 70 |
| CHK2(h) | 105 | 111 | 106 | 27 | 87 |
| CHK2(I157T)(h) | 103 | 104 | 93 | 96 | 64 |
| CHK2(R145W)(h) | 97 | 94 | 93 | 96 | 64 |
| CK1(y) | 113 | 117 | 108 | 111 | 83 |
| CK1γ1(h) | 100 | 101 | 97 | 83 | 38 |
| CK1γ2(h) | 99 | 96 | 91 | 69 | 35 |
| CK1γ3(h) | 106 | 101 | 95 | 63 | 43 |
| CK1δ(h) | 121 | 102 | 95 | 85 | 35 |
| cKit(D816H)(h) | 124 | 109 | 113 | 115 | 95 |
| cKit(D816V)(h) | 113 | 108 | 101 | 86 | 56 |
| cKit(h) | 105 | 110 | 79 | 97 | 89 |
| cKit(V560G)(h) | 115 | 109 | 107 | 104 | 91 |
| cKit(V654A)(h) | 108 | 110 | 103 | 103 | 85 |
| CLK2(h) | 108 | 104 | 104 | 98 | 93 |
| cSRC(h) | 103 | 98 | 97 | 97 | 83 |
| DAPK1(h) | 76 | 49 | 32 | 27 | 22 |
| DAPK2(h) | 66 | 61 | 51 | 53 | 36 |
| DCAMKL2(h) | 101 | 100 | 95 | 96 | 76 |
| DRAK1(h) | 104 | 103 | 99 | 90 | 79 |
| EGFR(L858R)(h) | 114 | 107 | 107 | 97 | 67 |
| EGFR(L861Q)(h) | 113 | 109 | 104 | 95 | 62 |
| EGFR(T790M)(h) | 107 | 100 | 103 | 104 | 101 |
| EGFR(T790M, | 109 | 104 | 102 | 95 | 81 |
| L858R)(h) | | | | | |
| EphA1(h) | 107 | 107 | 110 | 119 | 87 |
| EphA2(h) | 90 | 89 | 107 | 83 | 73 |
| EphA3(h) | 105 | 104 | 96 | 85 | 73 |
| EphA8(h) | 113 | 99 | 109 | 104 | 98 |
| EphB1(h) | 109 | 116 | 128 | 172 | 120 |
| EphB3(h) | 111 | 71 | 58 | 51 | 58 |
| EphB4(h) | 102 | 98 | 95 | 103 | 108 |
| Fer(h) | 104 | 102 | 91 | 66 | 61 |
| Fes(h) | 137 | 132 | 121 | 117 | 43 |
| FGFR1(V561M)(h) | 94 | 96 | 97 | 94 | 97 |
| FGFR2(h) | 109 | 100 | 75 | 63 | 66 |
| FGFR2(N549H)(h) | 109 | 106 | 105 | 107 | 97 |
| FGFR3(h) | 89 | 95 | 73 | 63 | 46 |
| FGFR4(h) | 98 | 96 | 60 | 35 | 25 |
| Fgr(h) | 108 | 106 | 102 | 92 | 81 |
| Flt1(h) | 105 | 104 | 102 | 103 | 92 |
| F1t3(D835Y)(h) | 104 | 101 | 95 | 94 | 91 |
| Flt3(h) | 108 | 106 | 103 | 100 | 61 |
| Flt4(h) | 109 | 104 | 100 | 85 | 75 |
| Fms(h) | 111 | 114 | 121 | 122 | 96 |
| Fyn(h) | 111 | 111 | 107 | 104 | 67 |
| GRK7(h) | 98 | 100 | 95 | 97 | 103 |
| GSK3α(h) | 110 | 90 | 67 | 47 | 22 |
| GSK3β(h) | 102 | 96 | 66 | 45 | 36 |
| Haspin(h) | 89 | 84 | 89 | 96 | 63 |
| Hck(h) | 109 | 99 | 85 | 70 | 50 |
| HIPK2(h) | 108 | 105 | 112 | 106 | 88 |
| IKKα(h) | 101 | 113 | 110 | 121 | 105 |
| IKKβ(h) | 97 | 97 | 103 | 101 | 71 |
| IR(h) | 100 | 99 | 95 | 85 | 81 |
| IRAK1(h) | 109 | 111 | 112 | 112 | 112 |
| Itk(h) | 76 | 107 | 105 | 101 | 104 |
| JAK2(h) | 105 | 112 | 106 | 105 | 96 |
| JAK3(h) | 100 | 96 | 88 | 84 | 65 |
| JNK3(h) | 105 | 105 | 105 | 93 | 82 |
| Lck(h) | 104 | 102 | 105 | 101 | 91 |
| Lyn(h) | 153 | 145 | 151 | 119 | 49 |
| Lyn(m) | 91 | 88 | 108 | 118 | 113 |
| MAPK1(h), ERK1 | 89 | 90 | 76 | 55 | 63 |
| MAPKAP-K2(h) | 107 | 112 | 112 | 92 | 64 |
| MAPKAP-K3(h) | 100 | 98 | 103 | 105 | 98 |
| MARK1(h) | 99 | 90 | 16 | 96 | 61 |
| MELK(h) | 105 | 99 | 97 | 97 | 86 |
| Met(h) | 109 | 118 | 146 | 91 | 68 |
| MKK4(m) | 112 | 118 | 109 | 107 | 107 |
| MKK7β(h) | 12 | 19 | 40 | 94 | 81 |
| MLCK(h) | 93 | 98 | 101 | 94 | 96 |
| MRCKα(h) | 113 | 103 | 102 | 107 | 95 |
| MRCKβ(h) | 103 | 106 | 112 | 110 | 86 |
| MSK1(h) | 104 | 101 | 100 | 83 | 43 |
| MSK2(h) | 101 | 92 | 86 | 70 | 31 |
| MSSK1(h) | 115 | 105 | 51 | 31 | 41 |
| MST3(h) | 98 | 107 | 119 | 108 | 58 |
| MuSK(h) | 98 | 99 | 102 | 103 | 99 |
| NEK11(h) | 99 | 83 | 60 | 40 | 36 |
| NEK2(h) | 97 | 97 | 104 | 115 | 99 |
| NEK3(h) | 99 | 100 | 99 | 97 | 97 |
| NEK6(h) | 90 | 92 | 84 | 80 | 46 |
| NEK7(h) | 113 | 103 | 98 | 108 | 81 |
| NLK(h) | 108 | 103 | 99 | 96 | 98 |
| p70S6K(h) | 111 | 104 | 116 | 103 | 95 |
| PAK2(h) | 121 | 115 | 116 | 111 | 102 |
| PAK3(h) | 121 | 107 | 59 | 51 | 37 |
| PAK4(h) | 106 | 96 | 107 | 113 | 109 |
| PAK5(h) | 101 | 99 | 103 | 97 | 70 |
| PAK6(h) | 93 | 80 | 92 | 88 | 32 |
| PAR-1Bα(h) | 110 | 106 | 110 | 115 | 103 |
| PASK(h) | 109 | 105 | 122 | 117 | 102 |
| PDGFRα(D842V)(h) | 129 | 122 | 131 | 123 | 101 |
| PDGFRα(V561D)(h) | 113 | 114 | 121 | 117 | 103 |
| PDGFRβ(h) | 54 | 95 | 80 | 100 | 110 |
| PDK1(h) | 98 | 87 | 84 | 67 | 69 |
| PhKγ2(h) | 117 | 119 | 111 | 93 | 50 |
| PI 3-Kinaseβ(h) | 97 | 98 | 81 | 60 | 34 |
| PI 3-Kinaseδ(h) | 98 | 96 | 77 | 64 | 26 |
| PI 3-Kinaseδ(h) | 89 | 88 | 70 | 47 | 58 |
| Pim-1(h) | 108 | 111 | 107 | 110 | 54 |
| Pim-2(h) | 100 | 97 | 100 | 92 | 81 |
| Pim-3(h) | 103 | 97 | 98 | 102 | 99 |
| PKA(h) | 95 | 102 | 110 | 112 | 117 |
| PKBα(h) | 125 | 130 | 119 | 125 | 103 |
| PKBβ(h) | 97 | 99 | 85 | 67 | 36 |
| PKBγ(h) | 109 | 103 | 97 | 101 | 86 |
| PKCα(h) | 102 | 104 | 96 | 93 | 72 |
| PKCβI(h) | 46 | 42 | 55 | 97 | 81 |
| PKCβII(h) | 114 | 116 | 108 | 103 | 82 |
| PKCγ(h) | 112 | 110 | 106 | 98 | 71 |
| PKCδ(h) | 118 | 118 | 107 | 99 | 97 |
| PKCε(h) | 111 | 103 | 97 | 96 | 94 |
| PKCθ(h) | 99 | 92 | 104 | 102 | 98 |
| PKG1α(h) | 107 | 103 | 106 | 103 | 83 |
| Plk3(h) | 113 | 110 | 110 | 101 | 99 |
| PRAK(h) | 80 | 50 | 26 | 23 | 20 |
| PrKX(h) | 102 | 101 | 92 | 91 | 61 |
| PTK5(h) | 107 | 111 | 106 | 115 | 105 |
| Ret(V804L)(h) | 107 | 97 | 102 | 87 | 72 |
| Ret(h) | 117 | 107 | 108 | 114 | 83 |
| Ret(V804M)(h) | 106 | 100 | 107 | 117 | 92 |
| ROCK-I(h) | 110 | 110 | 98 | 106 | 80 |
| Ron(h) | 112 | 111 | 127 | 126 | 85 |
| Rsk1(h) | 100 | 97 | 102 | 68 | 44 |
| Rsk1(r) | 105 | 96 | 99 | 91 | 43 |
| Rsk2(h) | 105 | 100 | 92 | 69 | 40 |
| Rsk3(h) | 111 | 102 | 105 | 98 | 95 |
| Rsk4(h) | 88 | 78 | 68 | 51 | 24 |
| SAPK2b(h) | 103 | 112 | 92 | 116 | 116 |
| SAPK3(h) | 113 | 108 | 109 | 118 | 109 |
| SAPK4(h) | 117 | 115 | 109 | 114 | 114 |
| SGK(h) | 96 | 97 | 102 | 91 | 87 |
| SGK2(h) | 114 | 121 | 123 | 106 | 78 |
| SIK(h) | 105 | 99 | 97 | 91 | 46 |
| Src(1-530)(h) | 101 | 105 | 103 | 98 | 57 |
| SRPK1(h) | 95 | 46 | 51 | 55 | 38 |
| SRPK2(h) | 107 | 112 | 100 | 93 | 85 |
| Syk(h) | 92 | 107 | 96 | 69 | 77 |
| TBK1(h) | 94 | 95 | 92 | 90 | 77 |
| Tie2(h) | 111 | 103 | 73 | 59 | 45 |
| Tie2(R849W)(h) | 97 | 56 | 40 | 55 | 58 |
| Tie2(Y897S)(h) | 84 | 53 | 41 | 34 | 26 |
| TLK2(h) | 101 | 105 | 101 | 98 | 97 |
| TrkA(h) | 106 | 107 | 100 | 55 | 10 |
| TrkB(h) | 120 | 111 | 117 | 112 | 85 |
| TSSK1(h) | 106 | 103 | 93 | 92 | 81 |
| TSSK2(h) | 109 | 103 | 97 | 103 | 84 |
| Txk(h) | 114 | 101 | 102 | 104 | 113 |
| ULK2(h) | 113 | 109 | 108 | 106 | 109 |
| WNK3(h) | 104 | 105 | 109 | 113 | 115 |
| Yes(h) | 110 | 111 | 109 | 110 | 91 |
| ZAP-70(h) | 124 | 119 | 119 | 123 | 116 |
| ZIPK(h) | 108 | 102 | 98 | 96 | 77 |

TABLE 8

| | TH-2 | | | | |
|---|---|---|---|---|---|
| | | | | OG- | OG- |
| | OG- | OG- | OG- | 3307 @ | 3307 @ |
| | 3307 @ | 3307 @ | 3307 @ | 50 | 100 |
| | 1 μg/ml | 5 μg/ml | 25 μg/ml | μg/ml | μg/ml |
| Abl(H396P)(h) | 96 | 103 | 83 | 65 | 54 |
| Abl(M351T)(h) | 92 | 95 | 89 | 75 | 56 |
| Abl(Q252H)(h) | 96 | 96 | 66 | 68 | 47 |
| Abl(h) | 92 | 87 | 70 | 64 | 40 |
| Abl(m) | 98 | 91 | 69 | 32 | 48 |
| Abl(T315I)(h) | 102 | 98 | 79 | 68 | 58 |
| Abl(Y253F)(h) | 117 | 100 | 83 | 65 | 58 |
| ACK1(h) | 117 | 124 | 97 | 86 | 81 |
| ALK(h) | 80 | 59 | 21 | 21 | 23 |
| ALK4(h) | 109 | 104 | 97 | 83 | 63 |
| Arg(h) | 92 | 92 | 69 | 53 | 43 |
| Arg(m) | 100 | 101 | 103 | 88 | 62 |
| ARK5(h) | 103 | 104 | 99 | 77 | 74 |
| Aurora-A(h) | 65 | 24 | 71 | 57 | 54 |
| Axl(h) | 106 | 110 | 100 | 86 | 65 |
| Blk(m) | 108 | 115 | 96 | 72 | 53 |
| Bmx(h) | 88 | 90 | 89 | 79 | 61 |
| BrSK1(h) | 104 | 101 | 82 | 66 | 50 |
| BrSK2(h) | 99 | 90 | 84 | 83 | 72 |
| BTK(h) | 98 | 96 | 67 | 59 | 47 |
| CaMKI(h) | 96 | 93 | 73 | 48 | 48 |
| CaMKII(r) | 105 | 106 | 105 | 91 | 63 |
| CaMKIIβ(h) | 103 | 106 | 95 | 83 | 66 |
| CaMKIIγ(h) | 109 | 109 | 131 | 108 | 101 |
| CaMKIIδ(h) | 99 | 99 | 100 | 91 | 87 |
| CaMKIV(h) | 117 | 126 | 86 | 69 | 52 |
| CaMKIδ(h) | 95 | 88 | 101 | 105 | 98 |
| CDK1/cyclinB(h) | 111 | 111 | 90 | 86 | 74 |
| CDK2/cyclinA(h) | 114 | 124 | 95 | 98 | 87 |
| CDK2/cyclinE(h) | 95 | 101 | 93 | 71 | 54 |
| CDK3/cyclinE(h) | 102 | 104 | 120 | 103 | 84 |
| CDK5/p25(h) | 93 | 96 | 103 | 88 | 77 |
| CDK5/p35(h) | 105 | 98 | 106 | 80 | 90 |
| CDK6/cyclinD3(h) | 120 | 109 | 117 | 105 | 92 |
| CDK9/cyclin T1(h) | 99 | 102 | 78 | 63 | 49 |
| CHK2(h) | 107 | 107 | 105 | 108 | 97 |
| CHK2(I157T)(h) | 108 | 104 | 94 | 83 | 74 |
| CHK2(R145W)(h) | 110 | 112 | 99 | 86 | 80 |
| CK1(y) | 114 | 113 | 110 | 110 | 100 |
| CK1γ1(h) | 100 | 101 | 88 | 73 | 48 |
| CK1γ2(h) | 103 | 96 | 79 | 54 | 49 |
| CK1γ3(h) | 96 | 91 | 83 | 43 | 34 |
| CK1δ(h) | 105 | 112 | 108 | 80 | 53 |
| cKit(D816H)(h) | 123 | 116 | 104 | 114 | 108 |
| cKit(D816V)(h) | 109 | 105 | 98 | 82 | 87 |
| cKit(h) | 110 | 87 | 86 | 96 | 95 |
| cKit(V560G)(h) | 112 | 114 | 105 | 90 | 78 |
| cKit(V654A)(h) | 100 | 101 | 112 | 88 | 75 |
| CLK2(h) | 103 | 100 | 110 | 101 | 96 |
| cSRC(h) | 114 | 114 | 108 | 101 | 87 |
| DAPK1(h) | 63 | 40 | 32 | 25 | 19 |
| DAPK2(h) | 61 | 56 | 55 | 52 | 49 |
| DCAMKL2(h) | 96 | 100 | 110 | 96 | 86 |
| DRAK1(h) | 103 | 106 | 107 | 93 | 79 |
| EGFR(L858R)(h) | 109 | 117 | 104 | 90 | 70 |
| EGFR(L861Q)(h) | 98 | 91 | 94 | 99 | 93 |
| EGFR(T790M)(h) | 106 | 104 | 104 | 100 | 102 |
| EGFR(T790M, | 104 | 109 | 95 | 94 | 82 |
| L858R)(h) | | | | | |
| EphA1(h) | 117 | 116 | 108 | 99 | 85 |
| EphA2(h) | 102 | 104 | 105 | 99 | 92 |
| EphA3(h) | 93 | 91 | 98 | 87 | 84 |
| EphA8(h) | 118 | 99 | 112 | 101 | 100 |
| EphB1(h) | 127 | 82 | 144 | 195 | 105 |
| EphB3(h) | 80 | 71 | 69 | 62 | 57 |
| EphB4(h) | 106 | 112 | 116 | 109 | 98 |
| Fer(h) | 110 | 102 | 104 | 91 | 88 |
| Fes(h) | 140 | 120 | 105 | 99 | 86 |
| FGFR1(V561M)(h) | 105 | 107 | 95 | 79 | 75 |
| FGFR2(h) | 111 | 98 | 102 | 96 | 78 |
| FGFR2(N549H)(h) | 110 | 102 | 103 | 103 | 82 |
| FGFR3(h) | 94 | 92 | 67 | 64 | 51 |
| FGFR4(h) | 89 | 81 | 55 | 64 | 55 |
| Fgr(h) | 95 | 96 | 112 | 106 | 86 |
| Flt1(h) | 102 | 97 | 103 | 102 | 98 |
| Flt3(D835Y)(h) | 108 | 116 | 102 | 105 | 98 |
| Flt3(h) | 107 | 99 | 109 | 95 | 84 |
| Flt4(h) | 117 | 113 | 88 | 80 | 55 |
| Fms(h) | 116 | 103 | 121 | 117 | 98 |
| Fyn(h) | 103 | 105 | 103 | 105 | 89 |
| GRK7(h) | 110 | 95 | 102 | 91 | 99 |
| GSK3α(h) | 94 | 92 | 61 | 51 | 43 |
| GSK3β(h) | 98 | 86 | 55 | 44 | 40 |
| Haspin(h) | 105 | 95 | 90 | 76 | 49 |
| Hck(h) | 103 | 88 | 76 | 62 | 42 |
| HIPK2(h) | 120 | 111 | 115 | 110 | 97 |
| IKKα(h) | 99 | 108 | 114 | 114 | 120 |
| IKKβ(h) | 111 | 107 | 94 | 77 | 76 |
| IR(h) | 96 | 100 | 80 | 75 | 79 |
| IRAK1(h) | 99 | 112 | 37 | 39 | 39 |
| Itk(h) | 101 | 103 | 87 | 82 | 87 |
| JAK2(h) | 108 | 119 | 101 | 95 | 89 |
| JAK3(h) | 74 | 67 | 62 | 53 | 35 |
| JNK3(h) | 110 | 108 | 80 | 92 | 88 |
| Lck(h) | 106 | 111 | 86 | 83 | 78 |
| Lyn(h) | 145 | 146 | 139 | 91 | 68 |
| Lyn(m) | 116 | 110 | 76 | 58 | 68 |
| MAPK1(h), ERK1 | 93 | 83 | 72 | 75 | 67 |
| MAPKAP-K2(h) | 116 | 104 | 104 | 74 | 73 |
| MAPKAP-K3(h) | 101 | 100 | 109 | 104 | 101 |
| MARK1(h) | 109 | 110 | 97 | 90 | 63 |
| MELK(h) | 116 | 104 | 102 | 98 | 83 |
| Met(h) | 114 | 113 | 97 | 83 | 61 |
| MKK4(m) | 120 | 127 | 91 | 95 | 85 |
| MKK7β(h) | 100 | 110 | 111 | 82 | 72 |
| MLCK(h) | 98 | 96 | 105 | 93 | 86 |
| MRCKα(h) | 100 | 94 | 112 | 102 | 97 |
| MRCKβ(h) | 110 | 114 | 109 | 104 | 99 |
| MSK1(h) | 98 | 103 | 92 | 64 | 52 |
| MSK2(h) | 99 | 94 | 70 | 51 | 37 |
| MSSK1(h) | 105 | 84 | 43 | 50 | 39 |
| MST3(h) | 107 | 89 | 87 | 46 | 45 |
| MuSK(h) | 104 | 101 | 99 | 98 | 97 |
| NEK11(h) | 97 | 106 | 96 | 75 | 51 |
| NEK2(h) | 106 | 109 | 105 | 102 | 72 |
| NEK3(h) | 99 | 102 | 97 | 94 | 92 |
| NEK6(h) | 103 | 100 | 79 | 67 | 53 |
| NEK7(h) | 103 | 107 | 98 | 95 | 84 |
| NLK(h) | 105 | 104 | 98 | 96 | 85 |
| p70S6K(h) | 108 | 108 | 108 | 92 | 69 |
| PAK2(h) | 116 | 110 | 107 | 101 | 96 |
| PAK3(h) | 103 | 102 | 49 | 47 | 40 |
| PAK4(h) | 105 | 84 | 98 | 110 | 99 |
| PAK5(h) | 106 | 105 | 91 | 93 | 61 |
| PAK6(h) | 85 | 113 | 90 | 89 | 42 |
| PAR-1Bα(h) | 109 | 113 | 114 | 116 | 88 |
| PASK(h) | 71 | 76 | 78 | 80 | 71 |
| PDGFRα(D842V)(h) | 113 | 124 | 128 | 129 | 127 |
| PDGFRα(V561D)(h) | 115 | 126 | 116 | 111 | 103 |
| PDGFRβ(h) | 111 | 110 | 53 | 50 | 44 |
| PDK1(h) | 122 | 123 | 102 | 87 | 69 |
| PhKγ2(h) | 112 | 116 | 114 | 88 | 63 |
| PI 3-Kinaseβ(h) | 99 | 95 | 75 | 47 | 28 |
| PI 3-Kinaseδ(h) | 95 | 100 | 72 | 42 | 19 |
| PI 3-Kinaseδ(h) | 97 | 89 | 62 | 18 | 52 |
| Pim-1(h) | 86 | 100 | 87 | 84 | 55 |
| Pim-2(h) | 103 | 111 | 77 | 73 | 67 |
| Pim-3(h) | 104 | 104 | 72 | 70 | 64 |
| PKA(h) | 131 | 124 | 93 | 94 | 86 |
| PKBα(h) | 123 | 130 | 126 | 118 | 79 |
| PKBβ(h) | 104 | 97 | 68 | 43 | 31 |
| PKBγ(h) | 112 | 110 | 107 | 97 | 83 |
| PKCα(h) | 103 | 99 | 98 | 98 | 71 |
| PKCβI(h) | 105 | 110 | 93 | 95 | 92 |
| PKCβII(h) | 107 | 105 | 99 | 103 | 98 |
| PKCγ(h) | 102 | 107 | 101 | 91 | 99 |
| PKCδ(h) | 111 | 110 | 97 | 92 | 80 |
| PKCε(h) | 105 | 102 | 98 | 102 | 90 |
| PKCθ(h) | 101 | 84 | 116 | 96 | 83 |
| PKG1α(h) | 103 | 112 | 97 | 92 | 73 |
| Plk3(h) | 120 | 105 | 104 | 99 | 86 |
| PRAK(h) | 66 | 45 | 19 | 24 | 19 |
| PrKX(h) | 101 | 103 | 98 | 88 | 66 |
| PTK5(h) | 116 | 105 | 113 | 112 | 116 |
| Ret(V804L)(h) | 110 | 103 | 91 | 81 | 60 |
| Ret(h) | 112 | 115 | 101 | 100 | 68 |
| Ret(V804M)(h) | 107 | 104 | 110 | 94 | 88 |
| ROCK-I(h) | 110 | 111 | 110 | 100 | 88 |
| Ron(h) | 123 | 124 | 123 | 116 | 94 |
| Rsk1(h) | 97 | 95 | 65 | 53 | 32 |
| Rsk1(r) | 101 | 102 | 93 | 63 | 36 |
| Rsk2(h) | 102 | 100 | 81 | 43 | 32 |
| Rsk3(h) | 112 | 106 | 97 | 88 | 85 |
| Rsk4(h) | 79 | 71 | 47 | 31 | 17 |
| SAPK2b(h) | 117 | 110 | 108 | 108 | 111 |
| SAPK3(h) | 109 | 99 | 114 | 122 | 106 |
| SAPK4(h) | 114 | 118 | 116 | 121 | 109 |
| SGK(h) | 106 | 96 | 97 | 92 | 76 |
| SGK2(h) | 133 | 116 | 112 | 121 | 69 |
| SIK(h) | 102 | 99 | 104 | 89 | 62 |
| Src(1-530)(h) | 103 | 105 | 105 | 102 | 75 |
| SRPK1(h) | 47 | 89 | 61 | 53 | 45 |
| SRPK2(h) | 105 | 104 | 99 | 91 | 88 |
| Syk(h) | 135 | 120 | 63 | 37 | 47 |
| TBK1(h) | 108 | 107 | 97 | 86 | 70 |
| Tie2(h) | 110 | 96 | 74 | 80 | 78 |
| Tie2(R849W)(h) | 91 | 53 | 48 | 43 | 46 |
| Tie2(Y897S)(h) | 82 | 46 | 27 | 35 | 32 |
| TLK2(h) | 98 | 89 | 106 | 94 | 98 |
| TrkA(h) | 110 | 111 | 99 | 31 | 25 |
| TrkB(h) | 136 | 132 | 144 | 118 | 78 |
| TSSK1(h) | 105 | 97 | 96 | 91 | 86 |
| TSSK2(h) | 106 | 99 | 100 | 97 | 87 |
| Txk(h) | 114 | 121 | 121 | 110 | 107 |
| ULK2(h) | 102 | 52 | 77 | 106 | 105 |
| WNK3(h) | 111 | 113 | 109 | 113 | 111 |
| Yes(h) | 92 | 81 | 116 | 111 | 104 |
| ZAP-70(h) | 125 | 110 | 124 | 124 | 107 |
| ZIPK(h) | 106 | 96 | 91 | 81 | 76 |

TABLE 9

| | TH 4 | | | | |
|---|---|---|---|---|---|
| | | | | OG- | OG- |
| | OG- | OG- | OG- | 3308 @ | 3308 @ |
| | 3308 @ | 3308 @ | 3308 @ | 50 | 100 |
| | 1 μg/ml | 5 μg/ml | 25 μg/ml | μg/ml | μg/ml |
| Abl (H396P)(h) | 106 | 102 | 73 | 35 | 11 |
| Abl(M351T)(h) | 96 | 98 | 68 | 40 | 11 |
| Abl(Q252H)(h) | 96 | 97 | 62 | 39 | 10 |
| Abl(h) | 87 | 84 | 52 | 33 | 0 |
| Abl(m) | 95 | 87 | 60 | 39 | 11 |
| Abl(T315I)(h) | 100 | 93 | 75 | 60 | 17 |
| Abl(Y253F)(h) | 105 | 96 | 69 | 49 | 16 |
| ACK1(h) | 100 | 103 | 95 | 96 | 58 |
| ALK(h) | 62 | 27 | 15 | 28 | 17 |
| ALK4(h) | 109 | 102 | 100 | 85 | 49 |
| Arg(h) | 85 | 79 | 61 | 26 | 10 |
| Arg(m) | 103 | 109 | 79 | 50 | 6 |
| ARK5(h) | 103 | 102 | 85 | 74 | 63 |
| Aurora-A(h) | 84 | 72 | 48 | 15 | 5 |
| Axl(h) | 118 | 106 | 94 | 75 | 56 |
| Blk(m) | 109 | 112 | 70 | 53 | 12 |
| Bmx(h) | 83 | 99 | 88 | 40 | 2 |
| BrSK1(h) | 107 | 80 | 71 | 38 | 15 |
| BrSK2(h) | 88 | 77 | 65 | 45 | 14 |
| BTK(h) | 99 | 98 | 64 | 14 | 4 |
| CaMKI(h) | 97 | 81 | 48 | 31 | 16 |
| CaMKII(r) | 103 | 104 | 101 | 82 | 38 |
| CaMKIIβ(h) | 100 | 98 | 81 | 35 | 9 |
| CaMKIIγ(h) | 100 | 103 | 107 | 92 | 80 |
| CaMKIIδ(h) | 97 | 100 | 83 | 66 | 51 |
| CaMKIV(h) | 130 | 125 | 103 | 86 | 44 |
| CaMKIδ(h) | 85 | 91 | 89 | 63 | 19 |
| CDK1/cyclinB(h) | 109 | 110 | 100 | 71 | 23 |
| CDK2/cyclinA(h) | 118 | 111 | 104 | 82 | 32 |
| CDK2/cyclinE(h) | 96 | 98 | 73 | 44 | 4 |
| CDK3/cyclinE(h) | 101 | 107 | 43 | 23 | 3 |
| CDK5/p25(h) | 86 | 88 | 76 | 46 | 2 |
| CDK5/p35(h) | 106 | 104 | 81 | 68 | 18 |
| CDK6/cyclinD3(h) | 104 | 103 | 105 | 99 | 4 |
| CDK9/cyclin T1(h) | 86 | 87 | 74 | 64 | 28 |
| CHK2(h) | 107 | 89 | 104 | 72 | 28 |
| CHK2(I157T)(h) | 101 | 97 | 75 | 53 | 20 |
| CHK2(R145W)(h) | 94 | 99 | 93 | 63 | 26 |
| CK1(y) | 111 | 106 | 104 | 91 | 21 |
| CK1γ1(h) | 88 | 91 | 58 | 25 | 12 |
| CK1γ2(h) | 92 | 90 | 55 | 20 | 9 |
| CK1γ3(h) | 89 | 87 | 51 | 39 | 8 |
| CK1δ(h) | 100 | 102 | 75 | 12 | 3 |
| cKit(D816H)(h) | 118 | 123 | 111 | 92 | 83 |
| cKit(D816V)(h) | 104 | 104 | 88 | 74 | 68 |
| cKit(h) | 97 | 94 | 94 | 89 | 90 |
| cKit(V560G)(h) | 120 | 111 | 99 | 52 | 34 |
| cKit(V654A)(h) | 96 | 98 | 89 | 74 | 53 |
| CLK2(h) | 101 | 101 | 99 | 93 | 23 |
| cSRC(h) | 101 | 87 | 101 | 88 | 35 |
| DAPK1(h) | 73 | 54 | 33 | 29 | 19 |
| DAPK2(h) | 69 | 67 | 57 | 39 | 44 |
| DCAMKL2(h) | 70 | 78 | 75 | 50 | 12 |
| DRAK1(h) | 101 | 102 | 90 | 77 | 52 |
| EGFR(L858R)(h) | 105 | 105 | 92 | 56 | 5 |
| EGFR(L861Q)(h) | 105 | 107 | 93 | 73 | 13 |
| EGFR(T790M)(h) | 105 | 112 | 106 | 104 | 15 |
| EGFR(T790M, | 105 | 98 | 93 | 84 | 52 |
| L858R)(h) | | | | | |
| EphA1(h) | 105 | 121 | 106 | 92 | 74 |
| EphA2(h) | 118 | 115 | 93 | 88 | 23 |
| EphA3(h) | 103 | 100 | 95 | 83 | 89 |
| EphA8(h) | 104 | 118 | 108 | 94 | 64 |
| EphB1(h) | 104 | 123 | 161 | 106 | 45 |
| EphB3(h) | 77 | 79 | 62 | 70 | 77 |
| EphB4(h) | 94 | 99 | 98 | 90 | 105 |
| Fer(h) | 89 | 87 | 83 | 75 | 35 |
| Fes(h) | 150 | 166 | 134 | 68 | 13 |
| FGFR1(V561M)(h) | 74 | 74 | 80 | 82 | 61 |
| FGFR2(h) | 86 | 84 | 61 | 53 | 35 |
| FGFR2(N549H)(h) | 107 | 104 | 106 | 95 | 25 |
| FGFR3(h) | 99 | 96 | 57 | 45 | 54 |
| FGFR4(h) | 111 | 85 | 41 | 24 | 25 |
| Fgr(h) | 103 | 104 | 105 | 69 | 2 |
| Flt1(h) | 97 | 105 | 100 | 94 | 87 |
| Flt3(D835Y)(h) | 99 | 101 | 102 | 85 | 13 |
| Flt3(h) | 103 | 107 | 95 | 62 | 59 |
| Flt4(h) | 104 | 91 | 95 | 67 | 29 |
| Fms(h) | 114 | 119 | 104 | 93 | 63 |
| Fyn(h) | 86 | 86 | 72 | 31 | 14 |
| GRK7(h) | 96 | 94 | 98 | 101 | 85 |
| GSK3α(h) | 85 | 80 | 40 | 12 | 2 |
| GSK3β(h) | 92 | 71 | 42 | 30 | −8 |
| Haspin(h) | 85 | 95 | 80 | 53 | 11 |
| Hck(h) | 99 | 93 | 63 | 38 | 6 |
| HIPK2(h) | 117 | 113 | 116 | 118 | 68 |
| IKKα(h) | 107 | 126 | 127 | 101 | 33 |
| IKKβ(h) | 111 | 117 | 104 | 72 | 23 |
| IR(h) | 95 | 104 | 87 | 97 | 93 |
| IRAK1(h) | 36 | 39 | 41 | 46 | 39 |
| Itk(h) | 81 | 86 | 82 | 91 | 65 |
| JAK2(h) | 104 | 97 | 98 | 87 | 76 |
| JAK3(h) | 77 | 69 | 58 | 28 | 4 |
| JNK3(h) | 111 | 101 | 98 | 78 | 53 |
| Lck(h) | 88 | 84 | 79 | 84 | 78 |
| Lyn(h) | 151 | 144 | 120 | 95 | 49 |
| Lyn(m) | 124 | 118 | 108 | 92 | 76 |
| MAPK1(h), ERK1 | 109 | 85 | 72 | 64 | 42 |
| MAPKAP-K2(h) | 111 | 108 | 88 | 59 | 28 |
| MAPKAP-K3(h) | 106 | 107 | 103 | 91 | 15 |
| MARK1(h) | 112 | 95 | 84 | 65 | 31 |
| MELK(h) | 101 | 97 | 90 | 88 | 49 |
| Met(h) | 120 | 121 | 89 | 62 | 18 |
| MKK4(m) | 104 | 112 | 99 | 107 | 76 |
| MKK7β(h) | 92 | 89 | 82 | 85 | 50 |
| MLCK(h) | 90 | 94 | 95 | 89 | 78 |
| MRCKα(h) | 97 | 96 | 99 | 81 | 49 |
| MRCKβ(h) | 111 | 113 | 109 | 85 | 13 |
| MSK1(h) | 106 | 105 | 72 | 51 | 8 |
| MSK2(h) | 87 | 92 | 51 | 37 | 11 |
| MSSK1(h) | 103 | 63 | 49 | 50 | 45 |
| MST3(h) | 110 | 119 | 74 | 51 | 15 |
| MuSK(h) | 110 | 87 | 82 | 82 | 81 |
| NEK11(h) | 97 | 80 | 42 | 33 | 33 |
| NEK2(h) | 94 | 45 | 50 | 45 | 17 |
| NEK3(h) | 94 | 90 | 86 | 68 | 38 |
| NEK6(h) | 64 | 60 | 48 | 21 | 7 |
| NEK7(h) | 103 | 108 | 101 | 75 | 31 |
| NLK(h) | 100 | 106 | 95 | 88 | 70 |
| p70S6K(h) | 100 | 96 | 90 | 70 | 67 |
| PAK2(h) | 108 | 101 | 109 | 94 | 34 |
| PAK3(h) | 110 | 81 | 41 | 32 | 11 |
| PAK4(h) | 107 | 119 | 99 | 93 | 70 |
| PAK5(h) | 98 | 104 | 109 | 70 | 12 |
| PAK6(h) | 92 | 97 | 64 | 19 | −3 |
| PAR-1Bα(h) | 108 | 104 | 98 | 78 | 54 |
| PASK(h) | 73 | 75 | 77 | 60 | 8 |
| PDGFRα(D842V)(h) | 135 | 117 | 110 | 93 | 56 |
| PDGFRα(V561D)(h) | 124 | 116 | 105 | 86 | 55 |
| PDGFRβ(h) | 59 | 55 | 71 | 69 | 91 |
| PDK1(h) | 123 | 114 | 94 | 87 | 60 |
| PhKγ2(h) | 84 | 84 | 57 | 25 | 6 |
| PI 3-Kinaseβ(h) | 51 | 100 | 37 | 27 | 11 |
| PI 3-Kinaseδ(h) | 98 | 97 | 49 | 33 | 20 |
| PI 3-Kinaseδ(h) | 34 | 92 | 40 | 50 | 35 |
| Pim-1(h) | 86 | 92 | 82 | 52 | 15 |
| Pim-2(h) | 80 | 76 | 71 | 64 | 40 |
| Pim-3(h) | 71 | 73 | 72 | 69 | 51 |
| PKA(h) | 106 | 115 | 123 | 107 | 29 |
| PKBα(h) | 84 | 97 | 86 | 67 | 52 |
| PKBβ(h) | 87 | 93 | 66 | 39 | 6 |
| PKBγ(h) | 116 | 114 | 115 | 104 | 80 |
| PKCα(h) | 99 | 95 | 91 | 84 | 66 |
| PKCβI(h) | 104 | 105 | 92 | 94 | 104 |
| PKCβII(h) | 109 | 108 | 98 | 105 | 99 |
| PKCγ(h) | 100 | 103 | 93 | 97 | 80 |
| PKCδ(h) | 102 | 105 | 97 | 95 | 94 |
| PKCε(h) | 103 | 104 | 96 | 98 | 98 |
| PKCθ(h) | 89 | 92 | 101 | 90 | 51 |
| PKG1α(h) | 97 | 94 | 94 | 64 | 31 |
| Plk3(h) | 110 | 111 | 110 | 100 | 92 |
| PRAK(h) | 61 | 35 | 20 | 33 | 24 |
| PrKX(h) | 96 | 93 | 81 | 49 | 2 |
| PTK5(h) | 110 | 108 | 106 | 106 | 31 |
| Ret(V804L)(h) | 110 | 95 | 78 | 57 | 37 |
| Ret(h) | 111 | 115 | 98 | 70 | 23 |
| Ret(V804M)(h) | 111 | 128 | 118 | 90 | 46 |
| ROCK-I(h) | 107 | 109 | 102 | 90 | 46 |
| Ron(h) | 115 | 120 | 120 | 93 | 31 |
| Rsk1(h) | 89 | 101 | 62 | 18 | 4 |
| Rsk1(r) | 95 | 97 | 59 | 25 | 2 |
| Rsk2(h) | 102 | 101 | 46 | 21 | 7 |
| Rsk3(h) | 111 | 113 | 100 | 85 | 65 |
| Rsk4(h) | 88 | 80 | 34 | 18 | 10 |
| SAPK2b(h) | 102 | 99 | 103 | 112 | 63 |
| SAPK3(h) | 94 | 93 | 96 | 91 | 55 |
| SAPK4(h) | 105 | 106 | 113 | 109 | 65 |
| SGK(h) | 91 | 90 | 93 | 61 | 12 |
| SGK2(h) | 114 | 115 | 97 | 61 | 15 |
| SIK(h) | 98 | 94 | 86 | 34 | 10 |
| Src(1-530)(h) | 101 | 100 | 88 | 38 | 5 |
| SRPK1(h) | 100 | 91 | 59 | 27 | 29 |
| SRPK2(h) | 103 | 110 | 88 | 87 | 61 |
| Syk(h) | 119 | 127 | 83 | 61 | 54 |
| TBK1(h) | 86 | 90 | 90 | 85 | 83 |
| Tie2(h) | 111 | 89 | 63 | 46 | 25 |
| Tie2(R849W)(h) | 77 | 43 | 67 | 49 | 46 |
| Tie2(Y897S)(h) | 71 | 44 | 25 | 16 | 9 |
| TLK2(h) | 98 | 97 | 96 | 91 | 78 |
| TrkA(h) | 93 | 103 | 38 | 12 | 7 |
| TrkB(h) | 114 | 130 | 129 | 53 | 18 |
| TSSK1(h) | 100 | 99 | 97 | 91 | 14 |
| TSSK2(h) | 104 | 101 | 105 | 84 | 17 |
| Txk(h) | 99 | 102 | 111 | 106 | 37 |
| ULK2(h) | 113 | 112 | 113 | 97 | 36 |
| WNK3(h) | 109 | 107 | 115 | 106 | 87 |
| Yes(h) | 113 | 113 | 112 | 94 | 8 |
| ZAP-70(h) | 71 | 67 | 61 | 56 | 33 |
| ZIPK(h) | 105 | 109 | 93 | 75 | 50 |

TABLE 10

| | TH-5 | | | | |
|---|---|---|---|---|---|
| | | | | OG- | OG- |
| | OG- | OG- | OG- | 3309 @ | 3309 @ |
| | 3309 @ | 3309 @ | 3309 @ | 50 | 100 |
| | 1 μg/ml | 5 μg/ml | 25 μg/ml | μg/ml | μg/ml |
| Abl(H396P)(h) | 112 | 103 | 79 | 45 | 10 |
| Abl(M351T)(h) | 104 | 105 | 72 | 38 | 8 |
| Abl(Q252H)(h) | 103 | 93 | 72 | 48 | 16 |
| Abl(h) | 97 | 80 | 58 | 25 | 2 |
| Abl(m) | 102 | 61 | 54 | 40 | 11 |
| Abl(T315I)(h) | 103 | 96 | 76 | 57 | 16 |
| Abl(Y253F)(h) | 100 | 99 | 70 | 51 | 14 |
| ACK1(h) | 116 | 115 | 97 | 87 | 46 |
| ALK(h) | 83 | 39 | 15 | 24 | 14 |
| ALK4(h) | 112 | 103 | 91 | 84 | 43 |
| Arg(h) | 100 | 95 | 73 | 27 | 11 |
| Arg(m) | 118 | 105 | 96 | 60 | 6 |
| ARK5(h) | 108 | 103 | 78 | 68 | 61 |
| Aurora-A(h) | 105 | 92 | 57 | 15 | 10 |
| Axl(h) | 113 | 114 | 94 | 81 | 53 |
| Blk(m) | 144 | 124 | 62 | 47 | 14 |
| Bmx(h) | 95 | 89 | 86 | 46 | 1 |
| BrSK1(h) | 102 | 95 | 75 | 43 | 12 |
| BrSK2(h) | 90 | 76 | 68 | 44 | 15 |
| BTK(h) | 108 | 100 | 62 | 15 | 5 |
| CaMKI(h) | 93 | 71 | 30 | 16 | 18 |
| CaMKII(r) | 109 | 113 | 98 | 70 | 43 |
| CaMKIIβ(h) | 107 | 104 | 72 | 24 | 9 |
| CaMKIIγ(h) | 126 | 111 | 100 | 86 | 68 |
| CaMKIIδ(h) | 103 | 102 | 82 | 63 | 58 |
| CaMKIV(h) | 120 | 123 | 96 | 93 | 42 |
| CaMKIδ(h) | 98 | 89 | 71 | 47 | 14 |
| CDK1/cyclinB(h) | 100 | 107 | 91 | 71 | 32 |
| CDK2/cyclinA(h) | 116 | 110 | 101 | 75 | 21 |
| CDK2/cyclinE(h) | 101 | 96 | 78 | 52 | 4 |
| CDK3/cyclinE(h) | 57 | 59 | 50 | 27 | 10 |
| CDK5/p25(h) | 101 | 84 | 85 | 57 | 5 |
| CDK5/p35(h) | 164 | 131 | 121 | 101 | 31 |
| CDK6/cyclinD3(h) | 121 | 107 | 92 | 82 | 13 |
| CDK9/cyclin T1(h) | 102 | 93 | 75 | 71 | 40 |
| CHK2(h) | 116 | 104 | 106 | 63 | 26 |
| CHK2(I157T)(h) | 108 | 98 | 88 | 51 | 18 |
| CHK2(R145W)(h) | 109 | 101 | 98 | 62 | 23 |
| CK1(y) | 126 | 114 | 100 | 82 | 19 |
| CK1γ1(h) | 97 | 91 | 70 | 34 | 20 |
| CK1γ2(h) | 103 | 98 | 65 | 23 | 17 |
| CK1γ3(h) | 103 | 96 | 49 | 33 | 11 |
| CK1δ(h) | 115 | 103 | 78 | 30 | 3 |
| cKit(D816H)(h) | 111 | 108 | 102 | 86 | 72 |
| cKit(D816V)(h) | 108 | 107 | 87 | 52 | 51 |
| cKit(h) | 117 | 99 | 92 | 94 | 89 |
| cKit(V560G)(h) | 111 | 106 | 96 | 69 | 28 |
| cKit(V654A)(h) | 101 | 97 | 87 | 70 | 56 |
| CLK2(h) | 111 | 108 | 105 | 87 | 18 |
| cSRC(h) | 109 | 99 | 90 | 79 | 38 |
| DAPK1(h) | 65 | 35 | 22 | 18 | 12 |
| DAPK2(h) | 71 | 61 | 54 | 41 | 37 |
| DCAMKL2(h) | 101 | 82 | 81 | 57 | 21 |
| DRAK1(h) | 111 | 107 | 93 | 77 | 59 |
| EGFR(L858R)(h) | 120 | 116 | 101 | 71 | 5 |
| EGFR(L86IQ)(h) | 110 | 106 | 104 | 75 | 17 |
| EGFR(T790M)(h) | 105 | 109 | 99 | 91 | 15 |
| EGFR(T790M, | 110 | 107 | 94 | 85 | 42 |
| L858R)(h) | | | | | |
| EphA1(h) | 115 | 112 | 110 | 82 | 69 |
| EphA2(h) | 125 | 128 | 106 | 98 | 14 |
| EphA3(h) | 113 | 100 | 96 | 92 | 92 |
| EphA8(h) | 115 | 116 | 106 | 94 | 76 |
| EphB1(h) | 106 | 120 | 159 | 102 | 45 |
| EphB3(h) | 80 | 69 | 56 | 51 | 50 |
| EphB4(h) | 103 | 104 | 93 | 84 | 97 |
| Fer(h) | 117 | 105 | 84 | 78 | 71 |
| Fes(h) | 171 | 150 | 108 | 56 | 15 |
| FGFR1(V561M)(h) | 84 | 79 | 75 | 73 | 53 |
| FGFR2(h) | 109 | 99 | 54 | 55 | 68 |
| FGFR2(N549H)(h) | 107 | 108 | 102 | 81 | 23 |
| FGFR3(h) | 94 | 90 | 50 | 65 | 65 |
| FGFR4(h) | 112 | 100 | 46 | 60 | 55 |
| Fgr(h) | 116 | 107 | 97 | 87 | 10 |
| Flt1(h) | 107 | 101 | 93 | 89 | 80 |
| Flt3(D835Y)(h) | 105 | 108 | 92 | 74 | 22 |
| Flt3(h) | 112 | 86 | 102 | 85 | 11 |
| Flt4(h) | 108 | 98 | 77 | 57 | 29 |
| Fms(h) | 114 | 118 | 112 | 99 | 69 |
| Fyn(h) | 89 | 80 | 76 | 45 | 12 |
| GRK7(h) | 104 | 92 | 87 | 88 | 86 |
| GSK3α(h) | 98 | 84 | 43 | 21 | 8 |
| GSK3β(h) | 91 | 68 | 43 | 34 | 15 |
| Haspin(h) | 79 | 73 | 67 | 39 | 17 |
| Hck(h) | 92 | 84 | 56 | 35 | 12 |
| HIPK2(h) | 120 | 117 | 107 | 104 | 65 |
| IKKα(h) | 124 | 131 | 125 | 108 | 50 |
| IKKβ(h) | 120 | 118 | 100 | 70 | 31 |
| IR(h) | 91 | 99 | 90 | 85 | 77 |
| IRAK1(h) | 43 | 39 | 41 | 43 | 39 |
| Itk(h) | 91 | 90 | 85 | 90 | 77 |
| JAK2(h) | 112 | 111 | 95 | 89 | 62 |
| JAK3(h) | 80 | 72 | 58 | 20 | 5 |
| JNK3(h) | 87 | 96 | 95 | 83 | 65 |
| Lck(h) | 86 | 91 | 85 | 75 | 63 |
| Lyn(h) | 154 | 149 | 126 | 95 | 49 |
| Lyn(m) | 92 | 98 | 77 | 100 | 65 |
| MAPK1(h), ERK1 | 108 | 93 | 75 | 62 | 44 |
| MAPKAP-K2(h) | 122 | 110 | 91 | 61 | 41 |
| MAPKAP-K3(h) | 113 | 110 | 106 | 100 | 25 |
| MARK1(h) | 109 | 97 | 83 | 60 | 29 |
| MELK(h) | 108 | 103 | 92 | 77 | 46 |
| Met(h) | 121 | 126 | 80 | 57 | 17 |
| MKK4(m) | 89 | 91 | 95 | 82 | 60 |
| MKK7β(h) | 109 | 90 | 71 | 62 | 37 |
| MLCK(h) | 101 | 104 | 91 | 84 | 70 |
| MRCKα(h) | 105 | 102 | 103 | 90 | 32 |
| MRCKβ(h) | 111 | 110 | 108 | 90 | 16 |
| MSK1(h) | 117 | 106 | 75 | 49 | 8 |
| MSK2(h) | 106 | 89 | 39 | 25 | 11 |
| MSSK1(h) | 114 | 75 | 45 | 53 | 16 |
| MST3(h) | 98 | 95 | 81 | 39 | 16 |
| MuSK(h) | 78 | 80 | 81 | 86 | 84 |
| NEK11(h) | 110 | 102 | 79 | 48 | 36 |
| NEK2(h) | 56 | 56 | 62 | 67 | 23 |
| NEK3(h) | 90 | 92 | 76 | 71 | 37 |
| NEK6(h) | 77 | 77 | 65 | 30 | 6 |
| NEK7(h) | 106 | 105 | 96 | 74 | 10 |
| NLK(h) | 112 | 108 | 91 | 84 | 75 |
| p70S6K(h) | 108 | 104 | 93 | 70 | 37 |
| PAK2(h) | 119 | 125 | 116 | 99 | 39 |
| PAK3(h) | 107 | 78 | 50 | 36 | 13 |
| PAK4(h) | 112 | 114 | 90 | 102 | 95 |
| PAK5(h) | 111 | 109 | 105 | 77 | 15 |
| PAK6(h) | 98 | 111 | 91 | 29 | 14 |
| PAR-1Bα(h) | 114 | 113 | 103 | 90 | 48 |
| PASK(h) | 68 | 70 | 73 | 71 | 15 |
| PDGFRα(D842V)(h) | 141 | 138 | 143 | 122 | 55 |
| PDGFRα(V561D)(h) | 109 | 119 | 116 | 90 | 43 |
| PDGFRβ(h) | 59 | 53 | 54 | 64 | 78 |
| PDK1(h) | 127 | 117 | 93 | 95 | 67 |
| PhKγ2(h) | 96 | 89 | 52 | 30 | 13 |
| PI 3-Kinaseβ(h) | 96 | 88 | 49 | 38 | 15 |
| PI 3-Kinaseδ(h) | 102 | 99 | 56 | 25 | 31 |
| PI 3-Kinaseδ(h) | 96 | 86 | 38 | 8 | 4 |
| Pim-1(h) | 95 | 92 | 75 | 44 | 21 |
| Pim-2(h) | 84 | 80 | 72 | 69 | 17 |
| Pim-3(h) | 73 | 67 | 65 | 65 | 71 |
| PKA(h) | 98 | 96 | 91 | 89 | 23 |
| PKBα(h) | 102 | 91 | 83 | 75 | 58 |
| PKBβ(h) | 112 | 108 | 63 | 37 | 6 |
| PKBγ(h) | 119 | 113 | 103 | 104 | 46 |
| PKCα(h) | 105 | 103 | 100 | 83 | 72 |
| PKCβI(h) | 102 | 102 | 95 | 84 | 72 |
| PKCβII(h) | 108 | 106 | 100 | 81 | 79 |
| PKCγ(h) | 98 | 96 | 99 | 68 | 71 |
| PKCδ(h) | 105 | 106 | 90 | 94 | 84 |
| PKCε(h) | 112 | 107 | 85 | 87 | 91 |
| PKCθ(h) | 100 | 92 | 89 | 89 | 71 |
| PKG1α(h) | 101 | 94 | 92 | 65 | 24 |
| Plk3(h) | 120 | 118 | 106 | 99 | 98 |
| PRAK(h) | 86 | 47 | 23 | 21 | 24 |
| PrKX(h) | 106 | 95 | 83 | 54 | 2 |
| PTK5(h) | 111 | 110 | 98 | 103 | 48 |
| Ret(V804L)(h) | 120 | 116 | 87 | 64 | 39 |
| Ret(h) | 117 | 105 | 98 | 74 | 28 |
| Ret(V804M)(h) | 133 | 129 | 119 | 97 | 46 |
| ROCK-I(h) | 123 | 119 | 100 | 91 | 50 |
| Ron(h) | 130 | 120 | 110 | 89 | 32 |
| Rsk1(h) | 109 | 101 | 71 | 36 | 5 |
| Rsk1(r) | 110 | 101 | 74 | 37 | 4 |
| Rsk2(h) | 113 | 103 | 44 | 36 | 12 |
| Rsk3(h) | 122 | 104 | 85 | 83 | 72 |
| Rsk4(h) | 96 | 82 | 37 | 20 | 7 |
| SAPK2b(h) | 105 | 117 | 104 | 101 | 70 |
| SAPK3(h) | 110 | 109 | 107 | 96 | 63 |
| SAPK4(h) | 110 | 116 | 108 | 104 | 74 |
| SGK(h) | 102 | 97 | 90 | 69 | 15 |
| SGK2(h) | 116 | 103 | 98 | 65 | 37 |
| SIK(h) | 106 | 97 | 82 | 38 | 8 |
| Src(1-530)(h) | 113 | 108 | 100 | 63 | 6 |
| SRPK1(h) | 101 | 92 | 51 | 42 | 19 |
| SRPK2(h) | 96 | 91 | 89 | 75 | 58 |
| Syk(h) | 104 | 78 | 43 | 61 | 64 |
| TBK1(h) | 104 | 93 | 80 | 67 | 51 |
| Tie2(h) | 89 | 90 | 77 | 72 | 71 |
| Tie2(R849W)(h) | 90 | 44 | 56 | 53 | 57 |
| Tie2(Y897S)(h) | 57 | 38 | 17 | 22 | 27 |
| TLK2(h) | 103 | 98 | 96 | 92 | 81 |
| TrkA(h) | 97 | 92 | 38 | 10 | 7 |
| TrkB(h) | 120 | 116 | 131 | 75 | 1 |
| TSSK1(h) | 105 | 103 | 94 | 84 | 20 |
| TSSK2(h) | 106 | 106 | 95 | 81 | 14 |
| Txk(h) | 111 | 109 | 102 | 99 | 66 |
| ULK2(h) | 73 | 111 | 105 | 103 | 93 |
| WNK3(h) | 108 | 112 | 114 | 113 | 80 |
| Yes(h) | 116 | 117 | 115 | 105 | 20 |
| ZAP-70(h) | 75 | 77 | 66 | 61 | 41 |
| ZIPK(h) | 110 | 93 | 87 | 80 | 34 |

TABLE 11

| | TH-7 | | | | |
|---|---|---|---|---|---|
| | | | | OG- | OG- |
| | OG- | OG- | OG- | 3310 @ | 3310 @ |
| | 3310 @ | 3310 @ | 3310 @ | 50 | 100 |
| | 1 μg/ml | 5 μg/ml | 25 μg/ml | μg/ml | μg/ml |
| Abl(H396P)(h) | 105 | 103 | 89 | 68 | 18 |
| Abl(M351T)(h) | 93 | 92 | 67 | 59 | 19 |
| Abl(Q252H)(h) | 95 | 92 | 65 | 55 | 32 |
| Abl(h) | 92 | 88 | 61 | 42 | 12 |
| Abl(m) | 94 | 83 | 57 | 47 | 11 |
| Abl(T1315I)(h) | 98 | 94 | 71 | 64 | 34 |
| Abl(Y253F)(h) | 101 | 103 | 74 | 59 | 26 |
| ACK1(h) | 120 | 128 | 116 | 97 | 79 |
| ALK(h) | 71 | 42 | 20 | 21 | 24 |
| ALK4(h) | 112 | 109 | 98 | 79 | 31 |
| Arg(h) | 106 | 95 | 76 | 43 | 21 |
| Arg(m) | 103 | 105 | 94 | 67 | 34 |
| ARK5(h) | 102 | 107 | 83 | 70 | 55 |
| Aurora-A(h) | 74 | 76 | 49 | 45 | 14 |
| AxI(h) | 108 | 103 | 90 | 84 | 22 |
| Blk(m) | 108 | 108 | 62 | 40 | 36 |
| Bmx(h) | 88 | 95 | 89 | 26 | 19 |
| BrSK1(h) | 102 | 97 | 83 | 62 | 10 |
| BrSK2(h) | 81 | 81 | 67 | 61 | 18 |
| BTK(h) | 102 | 92 | 60 | 44 | 9 |
| CaMKI(h) | 92 | 88 | 57 | 43 | 23 |
| CaMKII(r) | 105 | 112 | 96 | 80 | 54 |
| CaMKIIβ(h) | 99 | 101 | 87 | 69 | 44 |
| CaMKIIγ(h) | 91 | 105 | 100 | 106 | 97 |
| CaMKIIδ(h) | 96 | 104 | 85 | 82 | 76 |
| CaMKIV(h) | 122 | 125 | 98 | 74 | 48 |
| CaMKIδ(h) | 89 | 96 | 87 | 83 | 71 |
| CDK1/cyclinB(h) | 116 | 119 | 107 | 75 | 18 |
| CDK2/cyclinA(h) | 118 | 120 | 108 | 90 | 35 |
| CDK2/cyclinE(h) | 93 | 97 | 76 | 74 | 8 |
| CDK3/cyclinE(h) | 54 | 51 | 43 | 40 | 5 |
| CDK5/p25(h) | 77 | 79 | 72 | 79 | 7 |
| CDK5/p35(h) | 136 | 130 | 77 | 123 | 32 |
| CDK6/cyclinD3(h) | 115 | 104 | 88 | 174 | 80 |
| CDK9/cyclin T1(h) | 92 | 70 | 74 | 65 | 48 |
| CHK2(h) | 101 | 112 | 102 | 101 | 39 |
| CHK2(I157T)(h) | 105 | 104 | 88 | 75 | 14 |
| CHK2(R145W)(h) | 120 | 116 | 101 | 103 | 48 |
| CK1(y) | 105 | 116 | 101 | 85 | 30 |
| CK1γ1(h) | 98 | 97 | 73 | 46 | 15 |
| CK1γ2(h) | 96 | 100 | 59 | 40 | 11 |
| CK1γ3(h) | 101 | 96 | 49 | 34 | 4 |
| CK1δ(h) | 112 | 102 | 82 | 65 | 6 |
| cKit(D816H)(h) | 118 | 115 | 94 | 92 | 64 |
| cKit(D816V)(h) | 104 | 107 | 87 | 79 | 80 |
| cKit(h) | 97 | 97 | 72 | 70 | 79 |
| cKit(V560G)(h) | 103 | 107 | 88 | 69 | 45 |
| cKit(V654A)(h) | 94 | 102 | 77 | 70 | 47 |
| CLK2(h) | 109 | 103 | 99 | 97 | 74 |
| cSRC(h) | 100 | 108 | 109 | 90 | 31 |
| DAPK1(h) | 72 | 48 | 28 | 26 | 19 |
| DAPK2(h) | 67 | 74 | 51 | 53 | 45 |
| DCAMKL2(h) | 77 | 72 | 73 | 81 | 69 |
| DRAK1(h) | 103 | 105 | 93 | 83 | 73 |
| EGFR(L858R)(h) | 110 | 118 | 113 | 85 | 34 |
| EGFR(L861Q)(h) | 96 | 107 | 90 | 75 | 52 |
| EGFR(T790M)(h) | 107 | 109 | 104 | 98 | 65 |
| EGFR(T790M, | 103 | 99 | 101 | 87 | 63 |
| L858R)(h) | | | | | |
| EphA1(h) | 123 | 118 | 120 | 99 | 82 |
| EphA2(h) | 105 | 112 | 127 | 124 | 53 |
| EphA3(h) | 101 | 96 | 83 | 82 | 84 |
| EphA8(h) | 110 | 109 | 92 | 94 | 57 |
| EphB1(h) | 121 | 137 | 102 | 158 | 51 |
| EphB3(h) | 73 | 65 | 62 | 50 | 54 |
| EphB4(h) | 103 | 107 | 92 | 85 | 82 |
| Fer(h) | 98 | 97 | 80 | 91 | 75 |
| Fes(h) | 148 | 162 | 99 | 74 | 45 |
| FGFR1(V561M)(h) | 74 | 80 | 83 | 80 | 66 |
| FGFR2(h) | 93 | 91 | 68 | 72 | 59 |
| FGFR2(N549H)(h) | 108 | 112 | 102 | 86 | 48 |
| FGFR3(h) | 84 | 85 | 70 | 60 | 54 |
| FGFR4(h) | 113 | 80 | 44 | 65 | 62 |
| Fgr(h) | 103 | 103 | 100 | 97 | 47 |
| Flt1(h) | 101 | 101 | 96 | 91 | 25 |
| Flt3(D835Y)(h) | 95 | 96 | 99 | 100 | 60 |
| Flt3(h) | 116 | 107 | 101 | 98 | 34 |
| Flt4(h) | 106 | 101 | 86 | 68 | 17 |
| Fms(h) | 113 | 120 | 104 | 97 | 76 |
| Fyn(h) | 85 | 81 | 71 | 60 | 26 |
| GRK7(h) | 97 | 103 | 95 | 100 | 72 |
| GSK3α(h) | 81 | 88 | 52 | 39 | 7 |
| GSK3β(h) | 86 | 71 | 39 | 33 | 18 |
| Haspin(h) | 85 | 105 | 87 | 36 | 5 |
| Hck(h) | 92 | 82 | 60 | 41 | 11 |
| HIPK2(h) | 112 | 131 | 114 | 115 | 91 |
| IKKα(h) | 121 | 114 | 115 | 122 | 100 |
| IKKβ(h) | 110 | 114 | 100 | 86 | 32 |
| IR(h) | 79 | 53 | 30 | 43 | 49 |
| IRAK1(h) | 40 | 39 | 45 | 49 | 46 |
| Itk(h) | 87 | 84 | 81 | 96 | 86 |
| JAK2(h) | 108 | 106 | 102 | 91 | 75 |
| JAK3(h) | 72 | 64 | 51 | 71 | 9 |
| JNK3(h) | 103 | 103 | 100 | 80 | 60 |
| Lck(h) | 91 | 102 | 96 | 93 | 93 |
| Lyn(h) | 135 | 150 | 109 | 81 | 58 |
| Lyn(m) | 130 | 124 | 97 | 65 | 51 |
| MAPK1(h), ERK1 | 99 | 97 | 72 | 66 | 41 |
| MAPKAP-K2(h) | 121 | 114 | 79 | 62 | 43 |
| MAPKAP-K3(h) | 103 | 102 | 100 | 110 | 61 |
| MARK1(h) | 103 | 108 | 100 | 68 | 40 |
| MELK(h) | 96 | 101 | 99 | 83 | 56 |
| Met(h) | 130 | 133 | 73 | 68 | 23 |
| MKK4(m) | 104 | 110 | 123 | 79 | 75 |
| MKK7β(h) | 83 | 92 | 79 | 82 | 48 |
| MLCK(h) | 97 | 95 | 101 | 97 | 94 |
| MRCKα(h) | 99 | 105 | 91 | 96 | 66 |
| MRCKβ(h) | 113 | 117 | 107 | 95 | 41 |
| MSK1(h) | 102 | 113 | 70 | 54 | 28 |
| MSK2(h) | 70 | 73 | 38 | 32 | 16 |
| MSSK1(h) | 105 | 65 | 48 | 50 | 17 |
| MST3(h) | 97 | 98 | 62 | 35 | 29 |
| MuSK(h) | 94 | 83 | 87 | 75 | 73 |
| NEK11(h) | 92 | 101 | 94 | 71 | 41 |
| NEK2(h) | 45 | 45 | 46 | 52 | 18 |
| NEK3(h) | 74 | 67 | 66 | 88 | 51 |
| NEK6(h) | 71 | 67 | 54 | 41 | 8 |
| NEK7(h) | 105 | 106 | 96 | 87 | 40 |
| NLK(h) | 99 | 120 | 88 | 91 | 76 |
| p70S6K(h) | 101 | 97 | 100 | 74 | 28 |
| PAK2(h) | 115 | 114 | 110 | 106 | 86 |
| PAK3(h) | 95 | 76 | 47 | 48 | 26 |
| PAK4(h) | 103 | 89 | 66 | 100 | 74 |
| PAK5(h) | 118 | 105 | 99 | 76 | 33 |
| PAK6(h) | 98 | 86 | 85 | 71 | 27 |
| PAR-1Bα(h) | 104 | 99 | 92 | 97 | 81 |
| PASK(h) | 71 | 72 | 73 | 74 | 61 |
| PDGFRα(D842V)(h) | 113 | 123 | 122 | 125 | 96 |
| PDGFRα(V561D)(h) | 118 | 119 | 105 | 105 | 47 |
| PDGFRβ(h) | 61 | 69 | 55 | 47 | 56 |
| PDK1(h) | 126 | 129 | 95 | 68 | 55 |
| PhKγ2(h) | 87 | 86 | 60 | 39 | 22 |
| PI 3-Kinaseβ(h) | 99 | 95 | 34 | 4 | −4 |
| PI 3-Kinaseδ(h) | 99 | 90 | 44 | 11 | −1 |
| PI 3-Kinaseδ(h) | 89 | 87 | 24 | 12 | 9 |
| Pim-1(h) | 93 | 87 | 86 | 49 | 27 |
| Pim-2(h) | 83 | 77 | 81 | 68 | 43 |
| Pim-3(h) | 70 | 72 | 73 | 71 | 65 |
| PKA(h) | 115 | 119 | 115 | 79 | 44 |
| PKBα(h) | 94 | 81 | 89 | 76 | 47 |
| PKBβ(h) | 111 | 109 | 65 | 42 | 17 |
| PKBγ(h) | 116 | 123 | 113 | 97 | 41 |
| PKCα(h) | 99 | 102 | 96 | 97 | 60 |
| PKCβI(h) | 98 | 100 | 92 | 91 | 54 |
| PKCβII(h) | 105 | 106 | 100 | 93 | 62 |
| PKCγ(h) | 94 | 101 | 96 | 84 | 47 |
| PKCδ(h) | 107 | 102 | 87 | 71 | 79 |
| PKCε(h) | 103 | 100 | 87 | 86 | 76 |
| PKCθ(h) | 94 | 96 | 93 | 100 | 65 |
| PKG1α(h) | 98 | 99 | 100 | 75 | 36 |
| Plk3(h) | 115 | 106 | 97 | 94 | 84 |
| PRAK(h) | 38 | 48 | 27 | 20 | 16 |
| PrKX(h) | 96 | 100 | 87 | 63 | 34 |
| PTK5(h) | 103 | 108 | 106 | 110 | 65 |
| Ret(V804L)(h) | 113 | 107 | 81 | 66 | 43 |
| Ret(h) | 120 | 113 | 96 | 82 | 34 |
| Ret(V804M)(h) | 122 | 120 | 105 | 110 | 89 |
| ROCK-I(h) | 107 | 108 | 95 | 87 | 50 |
| Ron(h) | 118 | 121 | 117 | 97 | 48 |
| Rsk1(h) | 98 | 98 | 70 | 46 | 13 |
| Rsk1(r) | 101 | 96 | 69 | 38 | 8 |
| Rsk2(h) | 109 | 95 | 52 | 31 | 17 |
| Rsk3(h) | 121 | 108 | 85 | 87 | 62 |
| Rsk4(h) | 84 | 76 | 42 | 15 | 5 |
| SAPK2b(h) | 103 | 100 | 103 | 110 | 96 |
| SAPK3(h) | 92 | 92 | 105 | 116 | 91 |
| SAPK4(h) | 111 | 108 | 110 | 104 | 96 |
| SGK(h) | 93 | 101 | 93 | 75 | 31 |
| SGK2(h) | 114 | 100 | 87 | 83 | 35 |
| SIK(h) | 102 | 98 | 91 | 68 | 16 |
| Src(1-530)(h) | 109 | 116 | 107 | 95 | 7 |
| SRPK1(h) | 90 | 45 | 56 | 51 | 16 |
| SRPK2(h) | 94 | 87 | 84 | 83 | 72 |
| Syk(h) | 122 | 114 | 59 | 34 | 25 |
| TBK1(h) | 87 | 97 | 98 | 83 | 41 |
| Tie2(h) | 110 | 113 | 64 | 61 | 54 |
| Tie2(R849W)(h) | 81 | 42 | 38 | 50 | 46 |
| Tie2(Y897S)(h) | 69 | 43 | 31 | 28 | 25 |
| TLK2(h) | 95 | 92 | 83 | 94 | 74 |
| TrkA(h) | 85 | 95 | 34 | 7 | 16 |
| TrkB(h) | 128 | 132 | 139 | 79 | 49 |
| TSSK1(h) | 103 | 105 | 93 | 85 | 67 |
| TSSK2(h) | 102 | 103 | 101 | 92 | 51 |
| Txk(h) | 99 | 110 | 121 | 111 | 94 |
| ULK2(h) | 117 | 103 | 50 | 69 | 63 |
| WNK3(h) | 105 | 114 | 112 | 107 | 81 |
| Yes(h) | 110 | 110 | 105 | 107 | 58 |
| ZAP-70(h) | 74 | 58 | 52 | 65 | 57 |
| ZIPK(h) | 108 | 97 | 79 | 73 | 60 |

### Example 4

### Effect of Meta-THc on PI3K Activity

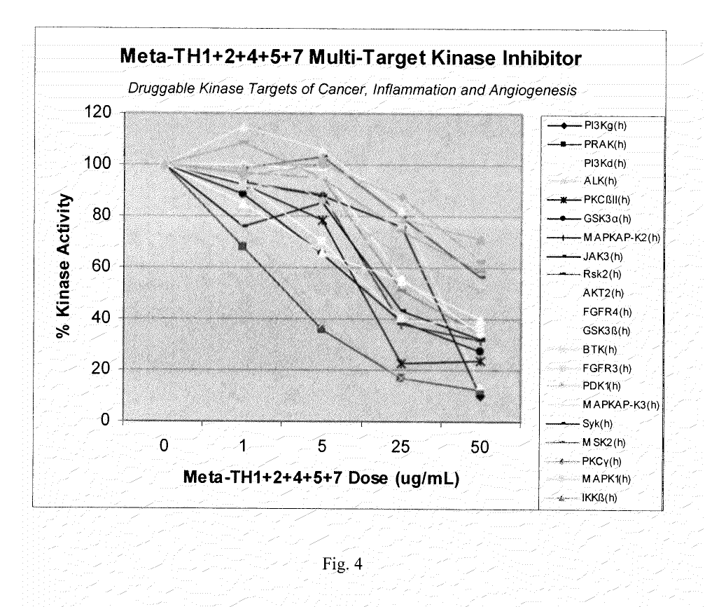

The inhibitory effect of Meta-THc on human PI3K-β, PI3K-γ, and PI3K-δ activity was examined according to the procedures and protocols of Example 1. All compounds were tested at at 1, 5, 25 and 50 μg/ml. The results are presented graphically as FIG. 4 comparing the kinase inhibition of PI3K activity as compared with test results against additional protein kinases implication in cancer, angiogenesis and inflammation.

### Example 5

### Inhibition of PGE₂ and Nitric Oxide by Meta-THc

LPS activated RAW 264.7 cells were assayed for PGE₂ and nitric oxide in the medium.

Materials—Meta-THc and its analogs were supplied by Metagenics (San Clemente, Calif.). LPS was purchased from Sigma (Sigma, St. Louis, Mo.). The concentration of Meta-THc was calculated based on the activities of cis and trans diastereomers of each of the three predominant n-, ad- and co-Meta-THc analogs. All other chemicals were of analytical grade purchased from Sigma (St. Louis, Mo.).

Cell Culture and Stimulation—The murine macrophage RAW 264.7 cell line was purchased from ATCC (Manassas, Va.) and maintained according to their instructions. Heat-inactivated fetal bovine serum (FBS), penicillin and streptomycin solution, and Dulbecco's Modification of Eagle's Medium (DMEM) were purchased from Mediatech (Herndon, Va.). Cells were grown and subcultured in 96-well plates at a density of 8×10⁴ cells per well reaching 90% confluence the next day. Test compounds were added to the cells in serum free medium at a final concentration of 0.1% dimethyl sulfoxide (DMSO). Following one hour of incubation with the test compounds, LPS (1 μg/ml) or DMEM medium alone was added to the cells and incubation continued for the indicated times. After the 4 hr stimulation with LPS, the media was collected and measured PGE₂ (Assay Designs, Ann Harbor, Mich.). For the measurement of nitric oxide production, the media was collected after 20 hr of LPS stimulation and nitratate/nitrite levels were measured (Cayman Chemicals, Ann Harbor, Mich.).

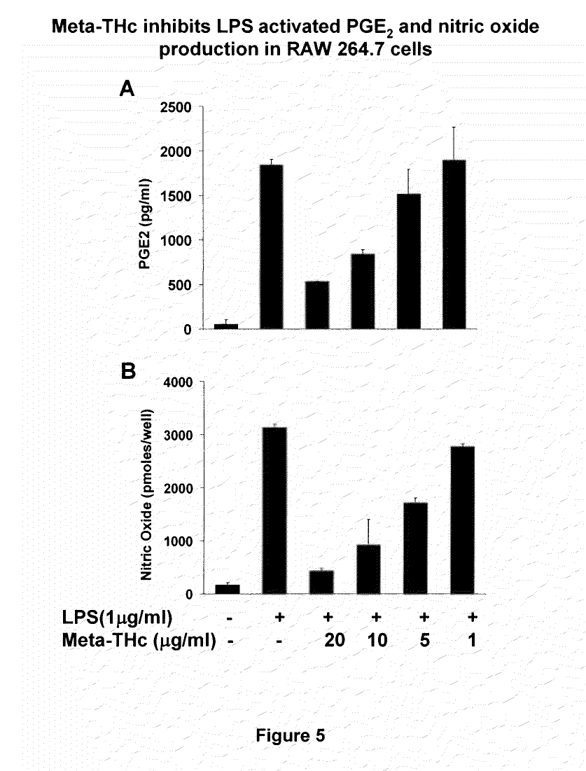

Results—Meta-THc inhibited PGE, and nitric oxide production in LPS activated RAW 264.7 cells and are presented in FIG. 5.

### Example 6

### Lack of Direct COX-2 Inhibition by Meta-THc

The objective was to determine the direct inhibition of COX-2 enzymatic activity.

Materials—Test compounds were prepared in DMSO and stored at −20° C. Meta-THc was supplied by Metagenics (San Clemente, Calif.). The commercial formulation of celecoxib (Celebrex®, G.D. Searle & Co., Chicago, Ill.) was used and all concentrations were based on the active material, although recipients were included. LPS was purchased from Sigma-Aldrich (St. Louis, Mo.).

Cell Culture—The murine macrophage RAW 264.7 cell line was purchased from ATCC (Manassas, Va.) and maintained according to their instructions. Cells were subcultured in 96-well plates at a density of 8×10⁴ cells per well and allowed to reach 90% confluence. LPS (1 μg/ml) or DMEM alone was added to the cell media and incubated for 20 hrs. Test compounds with LPS were added to the cells in serum free media at a final concentration of 0.1% DMSO. Following one hour of incubation with the test compounds, the cell media were removed and replaced with fresh media with test compounds with LPS (1 μg/ml) and incubated for 1 hr. The media were removed from the wells and analyzed for the PGE₂ synthesis.

PGE₂ assay—A commercial, non-radioactive procedure for quantification of PGE₂ was employed (Cayman Chemical, Ann Arbor, Mich.). Samples were diluted 10 times in EIA buffer and the recommended procedure of the manufacturer was used without modification. The PGE₂ concentration was represented as picograms per ml. The manufacturer's specifications for this assay include an intra-assay coefficient of variation of <10%, cross reactivity with PGD₂ and PGF₂ of less than 1% and linearity over the range of 10-1000 pg ml⁻¹.

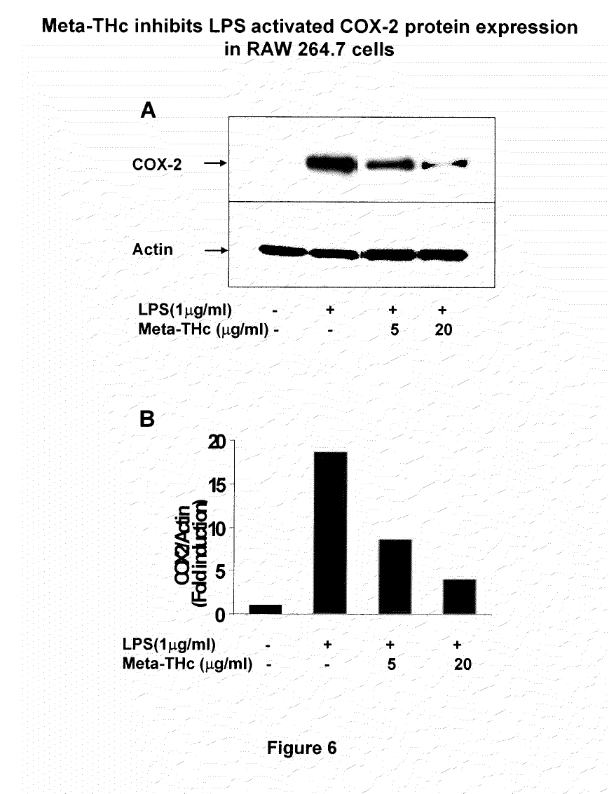

Results: The results indicate that Meta-THc was not a specific COX-2 enzymatic inhibitor and are presented in FIG. 6.

### Example 7

### Inhibition of COX-2 Protein by Meta-THc

Cellular extracts from RAW 264.7 cells stimulated with UPS were assayed for COX-2 protein by Western blot.

Materials—Test compounds were prepared in DMSO and stored at −20° C. Meta-THc was supplied by Metagenics (San Clemente, Calif.). Antibodies generated against COX-2 were purchased from Cayman Chemical (Ann Arbor, Mich.). Antibody generated against Actin was purchased from Sigma. Secondary antibodies coupled to horseradish peroxidase were purchased from Amersham Biosciences (Piscataway, N.J.).

Cell Culture—The murine macrophage RAW 264.7 cell line was purchased from ATCC (Manassas, Va.) and maintained according to their instructions. Test compounds were added to the cells in serum free medium at a final concentration of 0.1% DMSO. Following one hour of incubation with the test compounds, LPS (1 μg/ml) or DMEM alone was added to the cell wells and incubation continued for 16 hrs.

Western Blot analysis of COX-2: Cells were washed with cold PBS and lysed with 100 μl of lysis buffer (Bio-Rad). After denaturing, the samples were separated on SDS-PGE and transferred to nitrocellulose membrane. Incubation with the primary antibody followed by the secondary antibody was for one hr each at room temperature. Chemiluminescence was performed using the SuperSignal West Femto Maximum Sensitivity Substrate from Pierce Biotechnology (Rockford, Ill.) Western blot image was developed by autoradiogram (Kodak, BioMax film). Densitometry was performed using Kodak® software.

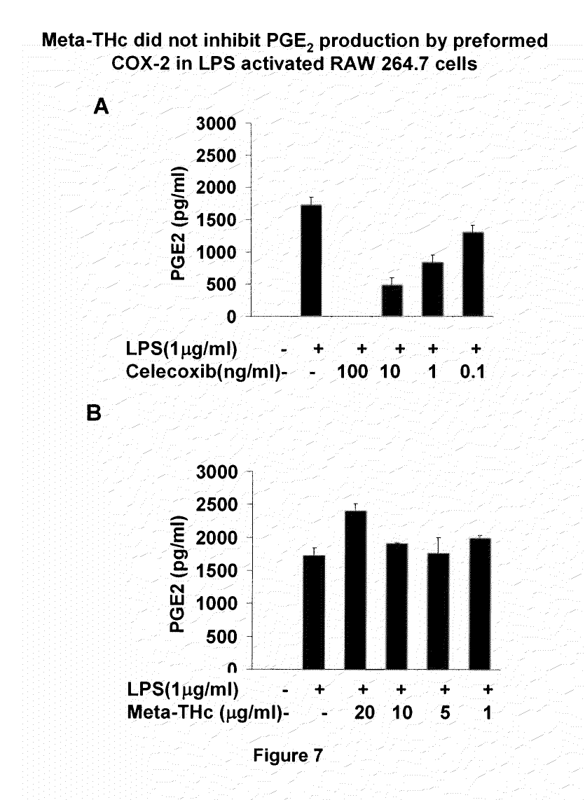

Results: The results indicated that Meta-THc inhibited COX-2 protein expression in LPS activated RAW 264.7 cells. The results are presented graphically in FIG. 7,

### Example 8

### NF-κB DNA Binding

Nuclear extracts from RAW 264.7 cells stimulated with LPS for 2 hours were assayed for NF-κB activity.

Materials—Test compounds were prepared in DMSO and stored at −20° C., Meta-THc was supplied by Metagenics (San Clemente, Calif.). Parthenolide was purchased from Sigma-Aldrich (St. Louis, Mo.).

Cell Culture—The murine macrophage RAW 264.7 cell line was purchased from ATCC (Manassas, Va.) and maintained according to their instructions. Cells were subcultured in 6-well plates at a density of 1.5×10⁶ cells per well and allowed to reach 90% confluence, approximately 2 days. Test compounds were added to the cells in serum free media at a final concentration of 0.1% DMSO. Following one hour of incubation with the test compounds, LPS (1 μg/ml) or DMEM alone was added to the cell media and incubation continued for an additional 2 hours.

NF-κB Binding—Nuclear extracts were prepared essentially as described by Dignam, et al [Nucl Acids Res 11:1475-1489, (1983)]. Briefly, cells are washed twice with cold PBS, then Buffer A (10 mM HEPES, pH 7.0; 1.5 mM MgCl₂; 10 mM KCl; 0.1% NP-40; aprotinin 5 μg/ml; pepstatin A 1 μg/ml; leupeptin 5 μg/ml; phenylmethanesulfonyl fluoride 1 mM) was added and allowed to sit on ice for 15 minutes. The lysis step was repeated with buffer A. The supernatant following centrifugation at 10,000×g for 5 minutes at 4° C. was the cytoplasmic fraction. The remaining pellet was resuspended in Buffer C (20 mM HEPES, pH 7.0; 1.5 mM KCl; 420 mM KCl; 25% glycerol; 0.2 M EDTA; aprotinin 5 μg/ml; pepstatin A 1 μg/ml; leupeptin 5 μg/ml; phenylmethanesulfonyl fluoride 1 mM) and sonicated (5×2 sec with 5 sec interval The nuclear extract fraction was collected as the supernatant following centrifugation at 10,000×g for 5 minutes at 4° C. DNA binding activity of the nuclear extracts was assessed using electrophoretic mobility shift assays (EMSA) with ATP (p32) labelled NF-κB consensus oligonucleotide (5′AGTTGAGGGGACTTTCCCAGGGC) Gel was exposed to autoradiography.

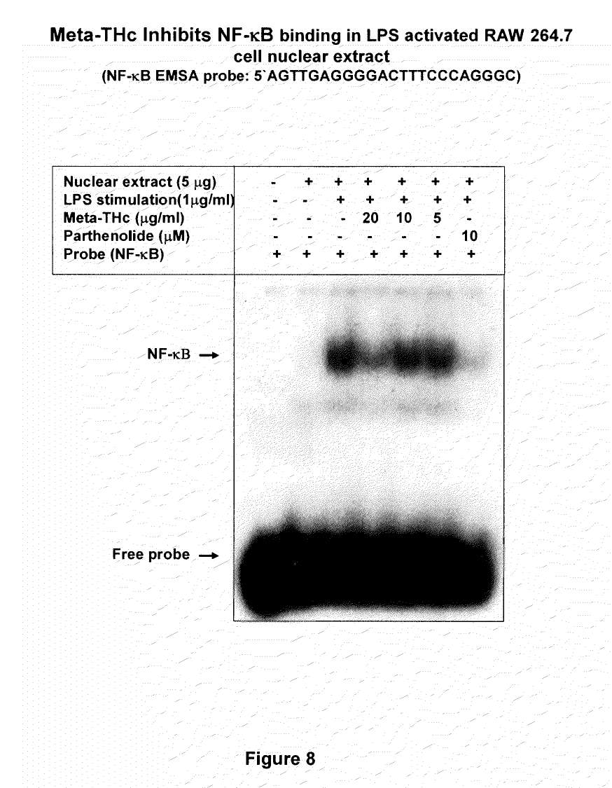

Results: The results indicated that Meta-THc inhibited nuclear translocation of NF-κB in LPS activated RAW 264.7 cells. The results are presented in FIG. 8.

### Example 9

### Inhibition of MMP-13 Expression

Human chondrosarcoma cells were assayed for MMP-13 secretion in the medium.

Materials—human TNFα and IL-113 were obtained from Sigma (St Louis, Mo.). The concentration of Meta-THc was calculated based on the activities of cis and trans diastereomers of each of the three predominant n-, ad- and co-Meta-THc analogs as well as other minor RIAA analogs. Assay kits for MMP-13 measurement were purchased from Amersham Biosciences (Piscataway, N.J.).

Cell culture: The human chondrocyte cell line, SW 1353 was purchased from ATCC (Manassas, Va.) and maintained in L-15 medium in the presence of 10% serum according to manufacturer instructions. Cells were grown and subcultured in 96-well plates at a density of 8×10⁴ cells per well and allowed to reach ˜80% confluence overnight, Test compounds in medium were added to the cells at a final concentration of 0.1% DMSO. Following one hour of incubation with the test compounds, TNFα (10 ng/ml) or IL-1β (10 ng/ml) or medium alone was added to the cell wells and incubation continued for 20-24 hours, The supernatant media was subsequently collected for MMP-13 determination (Amersham Biosciences, Piscataway, N.J.).

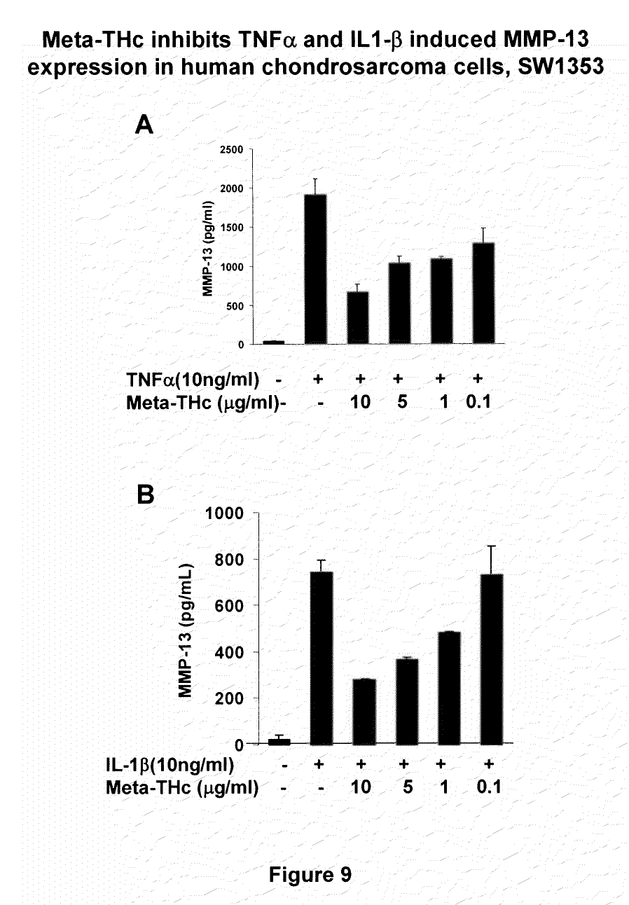

Results: Meta-THc dose dependently inhibited TNFα and IL-1β induced MMP-13 expression in SW 1353 cells. The results are presented as FIG. 9

### Example 10

### Inhibition of PGE₂ and Nitric Oxide by Meta-THc Analogs

LPS activated RAW 264.7 cells were assayed for PGE₂ and nitric oxide in the medium.

Materials—as described in Example 5

Cell Culture and Stimulation—as described in Example 5.

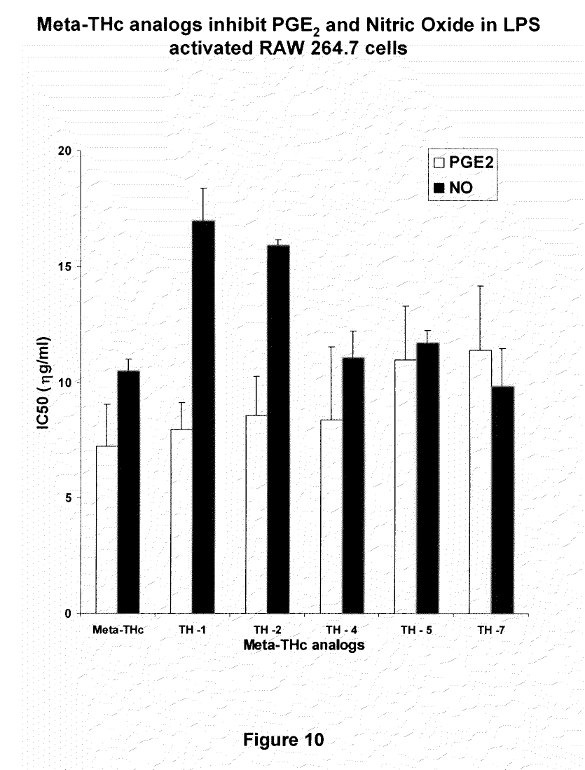

Results—Meta-THc analogs inhibited PGE₂ and nitric oxide production in LPS activated RAW 264.7 cells. The results are presented in FIG. 10.

### Example 11

### Meta-THc Analog Inhibition of Inflammation Associated Kinases

The objective was to determine whether Meta-THc components inhibit inflammation associated kinases.

Materials—as described in Example 1

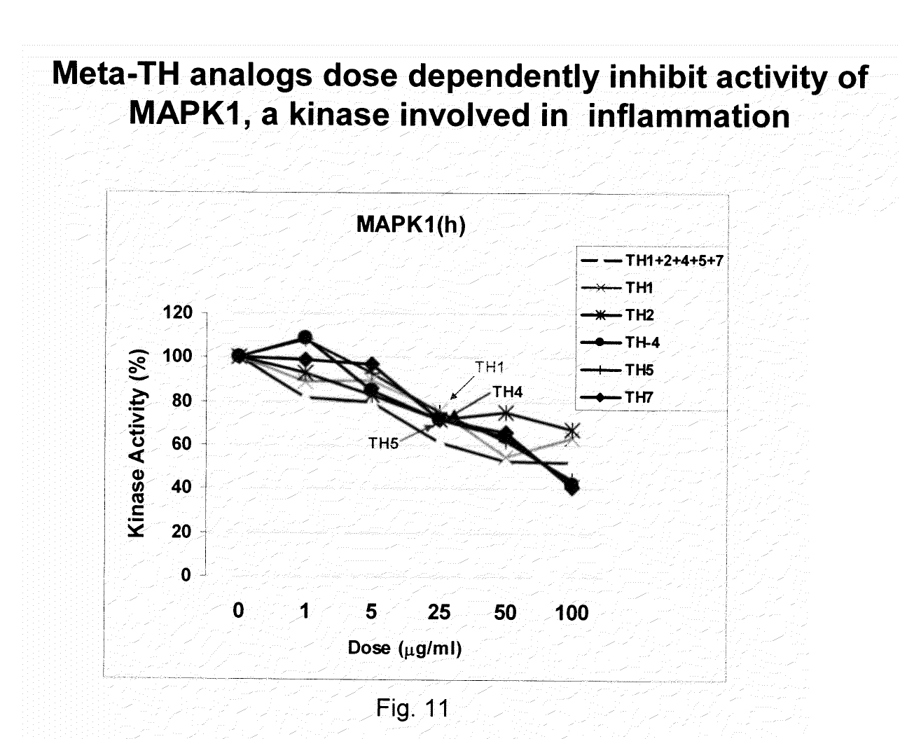

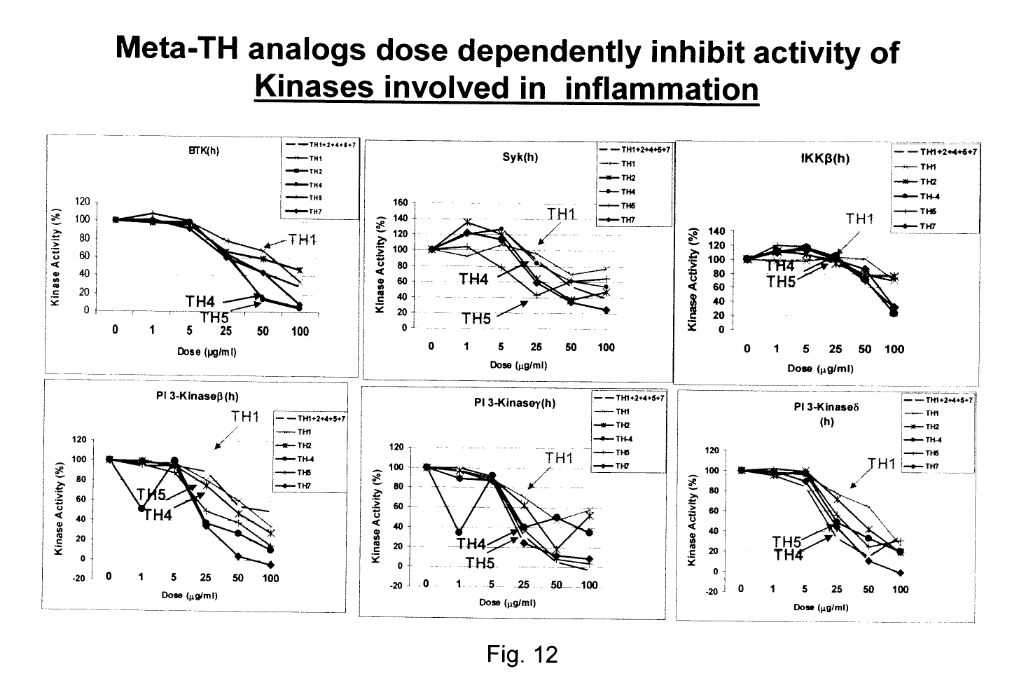

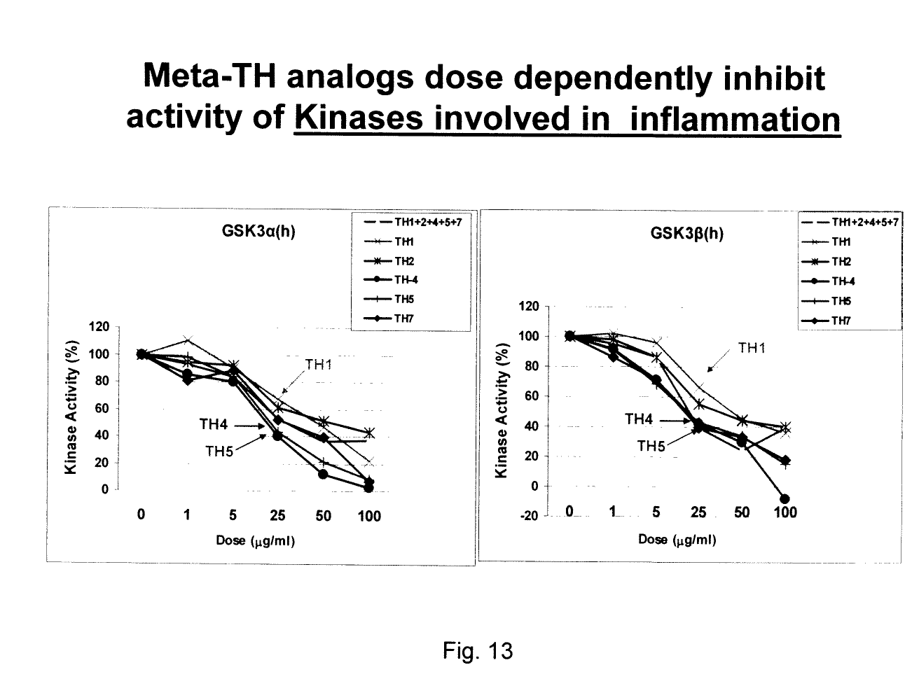

Results—The dose dependent inhibitory effects of Meta-THc components on selected kinases are presented in FIGS. 11-13.

### Example 12

### Meta-THc Analog Inhibition of Angiogenesis Associated Arg Tyrosine Kinase

The objective was to determine whether Meta-THc components inhibited the angiogenic associated ARG tyrosine kinase.

Materials—as described in Example 1.

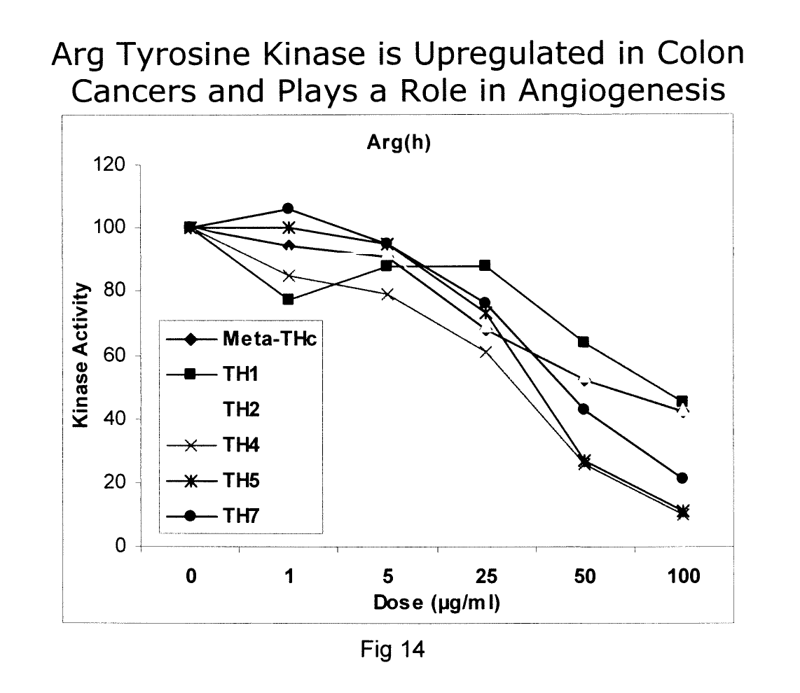

Results—The dose dependent inhibitory effects of Meta-THc components on selected kinases are presented in FIG. 14.

### Example 13

### Meta-THc Analog Inhibition of Colon Cancer Associated Kinases

The objective was to determine whether Meta-THc components inhibited the colon cancer associated kinases.

Materials—as described in Example 1.

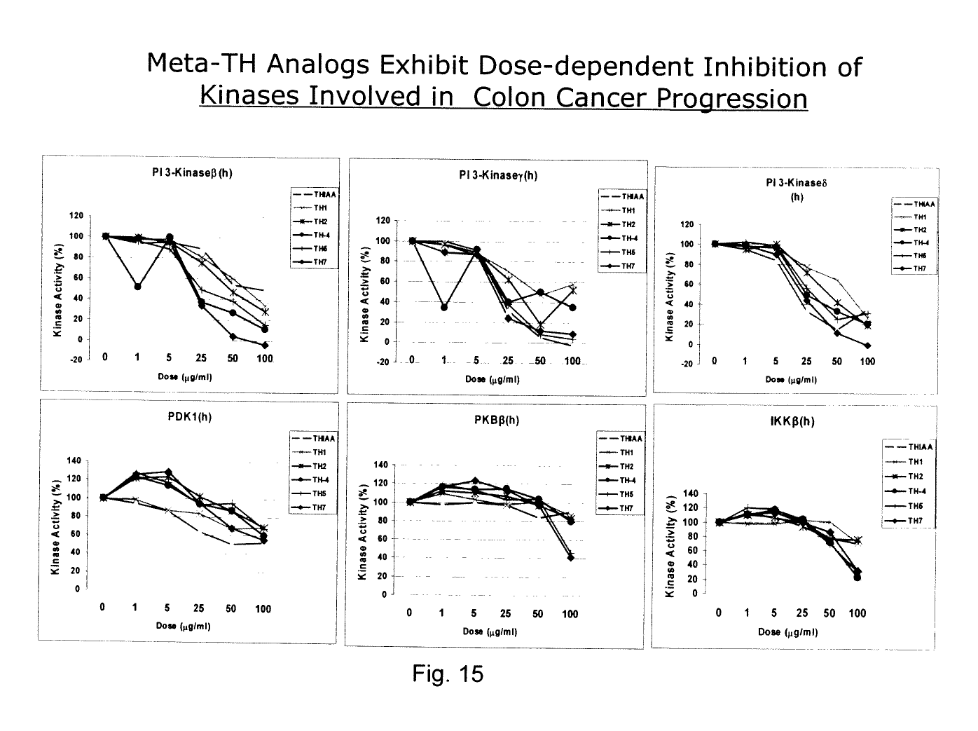

Results—The dose dependent inhibitory effects of Meta-THc components on selected kinases are presented in FIG. 15.

### Example 14

### Effects of Test Compounds in a Collagen Induced Rheumatoid Arthritis Murine Model

This example demonstrated the efficacy of Meta-THc in reducing inflammation and arthritic symptomology in a rheumatoid arthritis model, such inflammation and symptoms being known to mediated, in part, by a number of protein kinases.

The Model—Female DBA/J mice (10/group) were housed under standard conditions of light and darkness and allow diet ad libitum. The mice were injected intradermally on day 0 with a mixture containing 100 μg of type II collagen and 100 μg of Mycobacterium tuberculosis in squalene. A booster injection was repeated on day 21. Mice were examined on days 22-27 for arthritic signs with nonresponding mice removed from the study. Mice were treated daily by gavage with test compounds for 14 days beginning on day 28 and ending on day 42. Test compounds, as used in this example were Meta-THc at 10 mg/kg (lo), 50 mg/kg (med), or 250 mg/kg (hi); celecoxib at 20 mg/kg; and prednisolone at 10 mg/kg.

Arthritic symptomology was assessed (scored 0-4) for each paw using a arthritic index as described below. Under this arthritic index 0=no visible signs; 1=edema and/or erythema: single digit; 2=edema and or erythema: two joints; 3=edema and or erythema: more than two joints; and 4=severe arthritis of the entire paw and digits associated with ankylosis and deformity.

Histological examination—At the termination of the experiment, mice were euthanized and one limb, was removed and preserved in buffered formalin. After the analysis of the arthritic index was found to be encouraging, two animals were selected at random from each treatment group for histological analysis by H&E staining. Soft tissue, joint and bone changes were monitored on a four point scale with a score of 4 indicating severe damage.

Cytokine analysis—Serum was collected from the mice at the termination of the experiment for cytokine analysis. The volume of sample being low (˜0.2-0.3 ml/mouse), samples from the ten mice were randomly allocated into two pools of five animals each. This was done so to permit repeat analyses; each analysis was performed a minimum of two times. TNFα and IL-6 were analyzed using mouse specific reagents (R&D Systems, Minneapolis, Minn.) according to the manufacturer's instructions. Only five of the twenty-six pools resulted in detectable levels of TNF-α; the vehicle treated control animal group was among them.

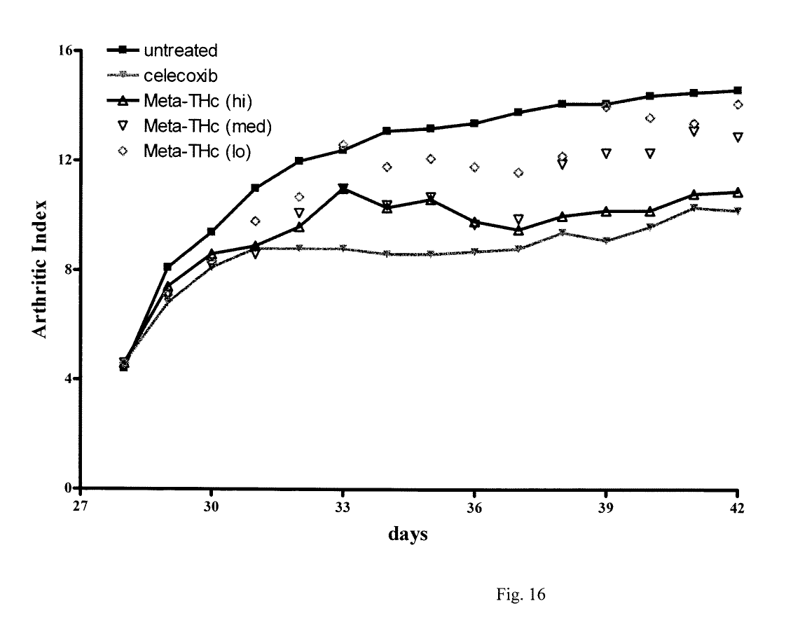

Results—FIG. 16 displays the effects of Meta-THc on the arthritic index. Here, significant reductions were observed for celecoxib (days 32-42), Meta-THc at 250 mg/kg (days 34-42) and Meta-THc at 50 mg/kg (days 34-40), also demonstrating the effectiveness of Meta-THc as an antiarthritic agent.

### Example 15

### Effects of Test Compounds on Cancer Cell Proliferation In Vitro

This experiment demonstrated the direct inhibitory effects on cancer cell proliferation in vitro for a number of Meta-THc test compounds of the instant invention.

Methods—The colorectal cancer cell lines HT-29, Caco-2 and SW480 were seeded into 96-well plates at 3×10³ cells/well and incubated overnight to allow cells to adhere to the plate. Each concentration of test material was replicated eight times. Seventy-two hours later, cells were assayed for total viable cells using the CyQUANT® Cell Proliferation Assay Kit. Percent decrease in viable cells relative to the DMSO solvent control was computed. Graphed values are means of eight observations ±95% confidence intervals.

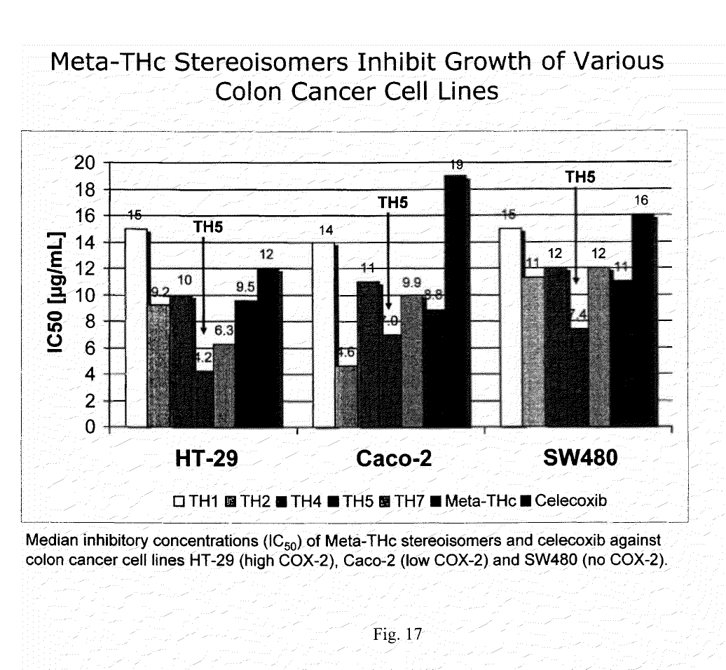

Results—FIG. 17 graphically presents the inhibitory effects of Meta-THc compounds.

### Example 16

### Detection of Meta-THc in Serum Following Oral Dosage

The purpose of this experiment was to determine whether Meta-THc was metabolized and detectable following oral dosage in humans.

Methods—Following a predose blood draw, five softgels (188 mg THIAA/softgel) delivering 940 mg of Meta-THc as the free acid (PR Tetra Standalone Softgel. OG#2210 KP-247. Lot C42331111) were consumed and immediately followed by a container of fruit yogurt. With the exception of decaffinated coffee, no additional food was consumed over the next four hours following Meta-THc ingestion. Samples were drawn at 45 minute intervals into Corvac Serum Separator tubes with no clot activator. Samples were allowed to clot at room temperature for 45 minutes and serum separated by centrifugation at 1800×g for 10 minutes at 4° C. To 0.3 ml of serum 0.9 ml of MeCN containing 0.5% HOAc was added and kept at −20° C. for 45-90 minutes. The mixture was centrifuged at 15000×g for 10 minutes at 4° C. Two phases were evident following centrifugation two phases were evident; 0.6 ml of the upper phase was sampled for HPLC analysis. Recovery was determined by using spiked samples and was greater than 95%.

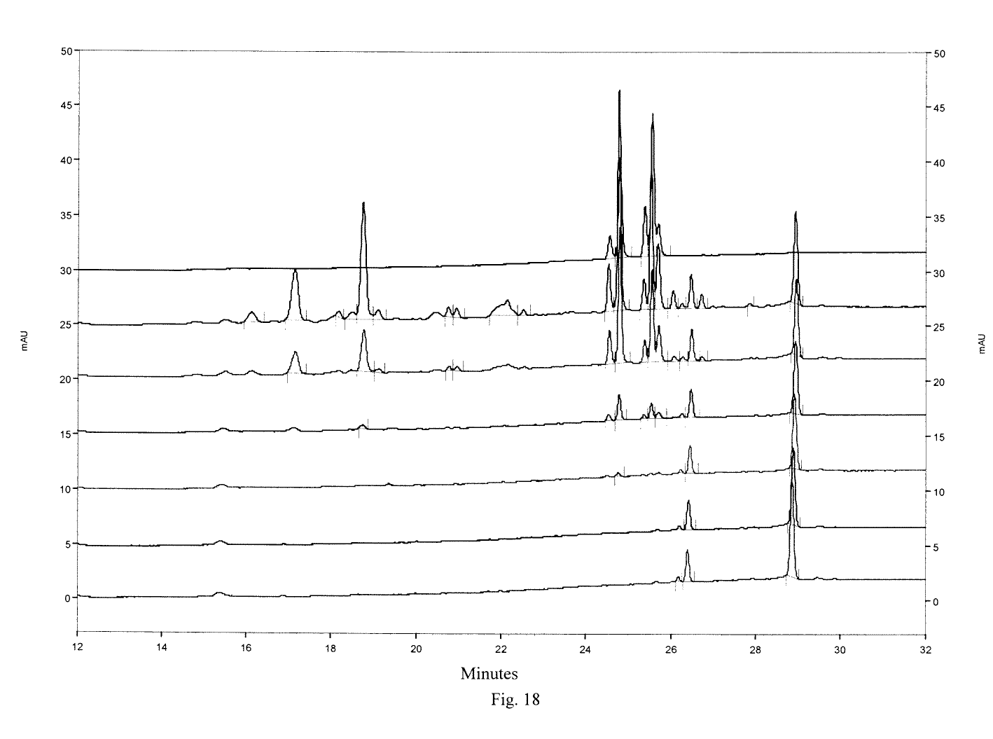

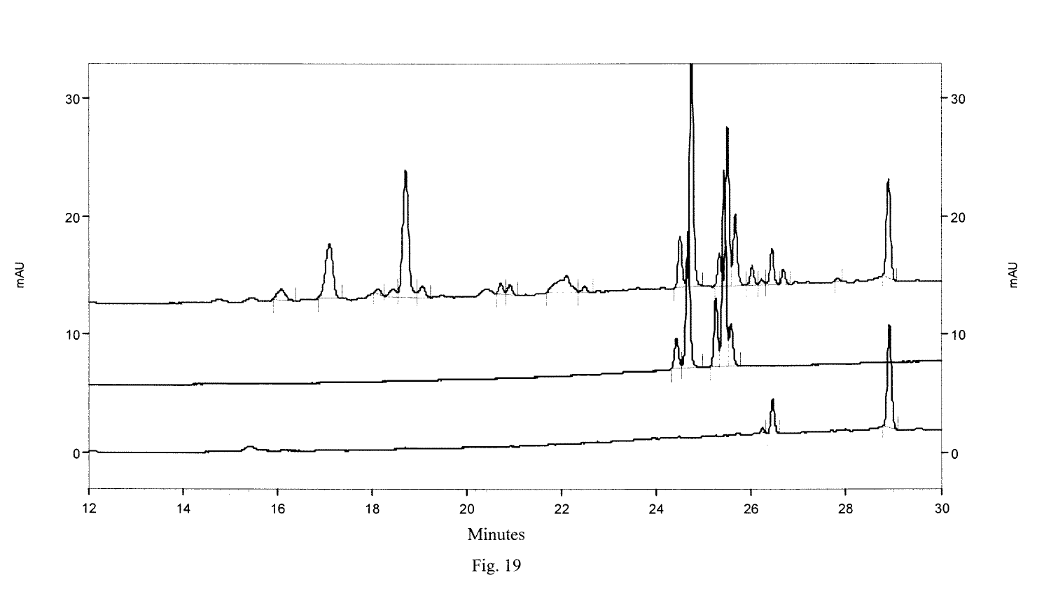

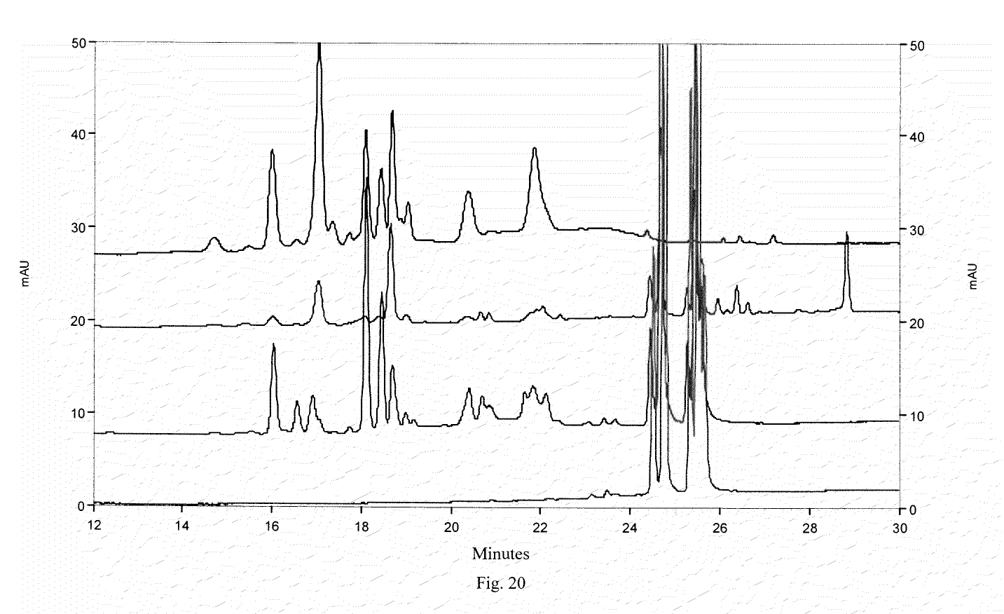

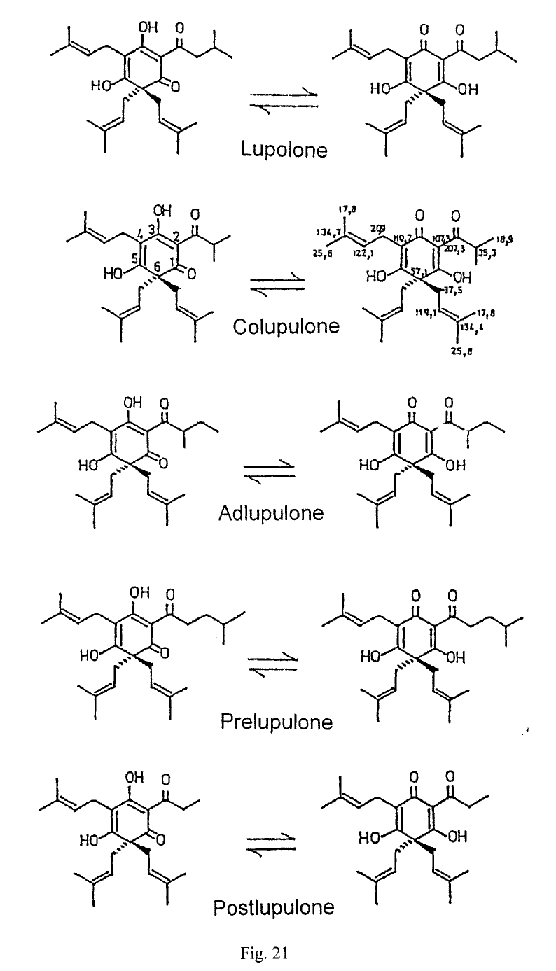

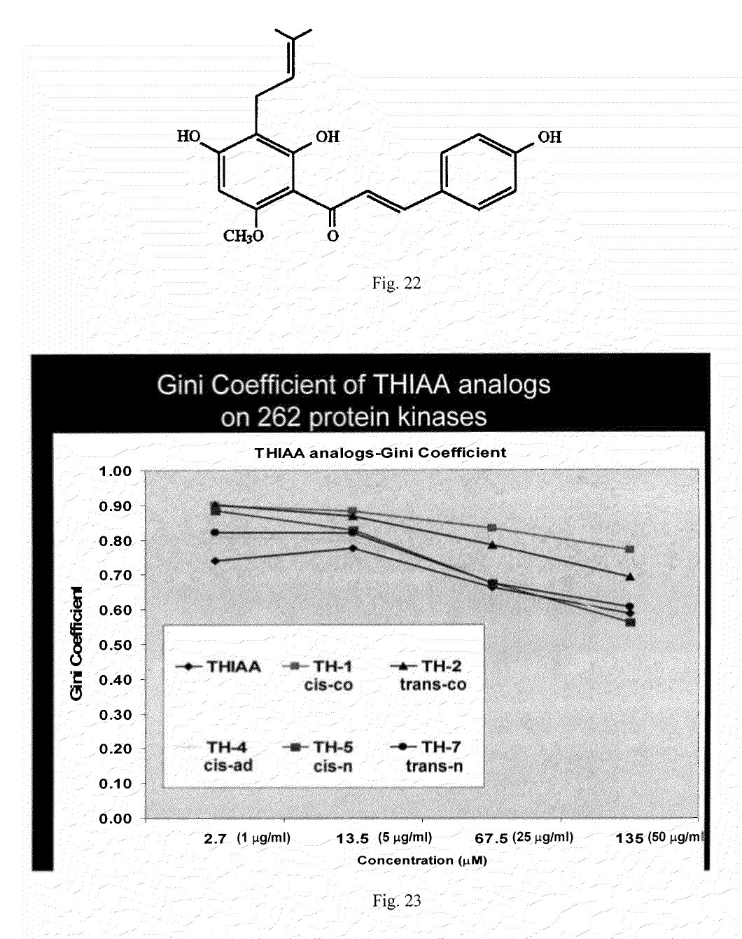

Results—The results are presented graphically as FIGS. 18-20. FIG. 18 graphically displays the detection of Meta-THc in the serum over time following ingestion of 940 mg of Meta-THc, FIG. 19 demonstrates that after 225 minutes following ingestion, Meta-THc was detected in the serum at levels comparable to those Meta-THc levels tested in vitro. FIG. 20 depicts the metabolism of Meta-THc by CYP2C9*1.

### Example 17

### Evaluation of the Anti-Angiogenic Activities of Hops Derivatives

Ex Vivo Rat Aortic Ring Angiogenesis Assay

Test materials and chemicals—The test materials isoalpha acid (IAA), rho-isoalpha acid (RIAA), tetrahydroisoalpha acid (THIAA), hexahydroisoalpha acid (HHIAA), beta acids (BA) and xanthohumol (XN) were supplied by Metaproteomics, Gig Harbor, Wash. All standard chemicals, media and reagents, unless otherwise noted, were purchased from Sigma, St. Louis, Mo.

Methodology—Cleaned rat aortic rings were embedded into rat tail interstitial type I collagen gel (1.5 mg/ml). This final collagen solution was obtained by mixing 7.5 volumes of type I collagen (2 mg/ml, Collagen R; Serva, Heidelberg, Germany) with 1 volume of 10 times concentrated DMEM, 1.5 volumes of sodium bicarbonate solution (15.6 mg/ml), and 0.1 volume of sodium hydroxide solution (1 M) to adjust the pH to 7.4. Collagen-embedded rat aortic rings were processed in cylindrical agarose wells and placed in triplicate in 60-mm bacteriologic polystyrene dishes containing 8 ml of serum-free MCDB-131 (Invitrogen) supplemented with 25 mM NaHCO3, 1% glutamine, 100 U/ml penicillin, and 100 g/ml streptomycin. These ex vivo organo-typic cultures were treated with single compound. After 9 days of culture at 37 C under an air-CO2 (95%:5%) atmosphere, the aortic rings were photographed under an optic microscope (25 magnification, Carl Zeiss AxioCam HR Workstation, KS100 3.0 software). Neovascularization was evaluated as a marker of the observed angiogenic response.

Statistical analysis—Analysis of variance was performed on the six observations per treatment for the controls and two test concentrations after normalizing to the dimethyl sulfoxide control. The probability of a type I error was set at the nominal five percent level.

TABLE 12
Relative Number of Vessels vs Dimethyl Sulfoxide Controls

Dose

| Test Material | 20 μg/mL | 5.0 μg/mL |
|---|---|---|
| Isoalpha acid | 113** | 108 |
| Rho-isoalpha acid | 16.2** | 75.2** |
| Tetrahydro isoalpha acid | 0.00** | 15.9** |
| Hexahydroisoalpha acid | 81.1** | 98.5 |
| Beta acids | 52.0** | 96.0 |
| Xanthohumol | 100 | 95.9 |

*p < 0.05;
**p < 0.01

Results—Both RIAA and THIAA effectively inhibited vessel growth at both 20 and 5 μgh/mL [SHOULD THIS BE 5 or 50 μg/mL?], while HHIAA and BA were active only at the 20 μg/mL concentration. Xanthohumol was not active in this assay and IAA actually increased vessel growth at the higher concentration.

Migration Wound Healing Assay

Methodology—A day before the assay, 5×10⁵ endothelial cells were plated in E-well plates and grown in adequate complete medium over night. Confluent HUVEC monolayers were then scraped to create a wound. Cells were the treated with 20 ug/ml of each drug. After wounding and 6 hours later, two different fields of each wound were photographed with a phase-contrast microscope. Measurements of the width of each wound were made in each experimental condition. At the start of the experiment, the wound size was measured and scored as 100%. After 6 hours, the width of the remaining wound was measured and average percent wound closure was calculated.

TABLE 13
Relative Percent Wound Closure at Six Hours vs
Dimethyl Sulfoxide Controls

| | Dose |
|---|---|
| Test Material | 20 μg/mL |
| Isoalpha acid | 74** |
| Rho-isoalpha acid | 80* |
| Tetrahydro isoalpha acid | 61** |
| Hexahydroisoalpha acid | 106 |
| Beta acids | 68** |
| Xanthohumol | 45** |

*p < 0.05;
**p < 0.01

Results—Of the six test materials, only HHIAA failed to inhibit wound closure. The most active of the test materials was XN, followed by THIAA, BA, IAA and RIAA.

Proliferation Assay

Methodology—A day before the assay, 1×10⁴ endothelial cells were plated in quadruplicate in 24-well plates ad grown in adequate complete medium over night. Cells were then treated with 10 ug/ml and 20 ug/ml of each drug. After 6 hours, 48 hours and 72 hours cells were then resuspended and sonicated in 200 ul PBS. 100 ul of the sonicated samples were transferred to 96-well microplates and 100 ul of Hoechst 33258 (2 ug/ml) was added. For the standard curve, 100 ul of DNA standards with concentrations of 0.3125, 0.625, 1.25, 2.15, 5, 10 and 20 ug/ml were used. The dilutions and concentrations of the dyes were chosen to yield appropriate dye/base pair ratios that are crucial to obtain maximal linearity and sensitivity of the DNA quantification assays. After an incubation time of ˜10 min, fluorescence intensities were measure. All investigations were generally performed at room temperature with the solutions protected from light. Spectrofluorimetric measurements were performed with Spectramax Gemini XS. Hoechst 33258 was excited at 360 nm and fluorescence emission was detected at 458 nm. Florescence values were converted into DNA concentrations according to the fluorescence intensities of DNA standard calibration curves.

TABLE 14
Relative DNA Content vs Dimethyl Sulfoxide Controls

| | | | 48 Hours | | 72 Hours | |
|---|---|---|---|---|---|---|
| | 24 Hours | | 10 | 20 | 10 | 20 |
| | 10 | 20 | μg/ | μg/ | μg/ | μg/ |
| Test Material | μg/mL | μg/mL | mL | mL | mL | mL |
| Isoalpha acid | 103 | 97 | 100 | 98 | 100 | 98 |
| Rho-isoalpha acid | 96 | 84** | 93* | 75** | 80** | 52** |
| Tetrahydro isoalpha | 95 | 82** | 88** | 79** | 79** | 40** |
| acid | | | | | | |
| Hexahydroisoalpha | 80** | 71** | 63** | 55** | 57** | 35** |
| acid | | | | | | |
| Beta acids | 103 | 97 | 100 | 98 | 98 | 97 |
| Xanthohumol | 95 | 66** | 82** | 57** | 71** | 42** |

*p < 0.05;
**p < 0.01

Results—HHIAA was most active among the test agents, inhibiting proliferation at both concentrations and all time points. THIAA and XN provided similar inhibition by 72 hours followed by RIAA. Neither BA nor IAA effectively inhibited proliferation in this assay.

Conclusions

TABLE 15
Summary of Effects

| | Assays | | | |
|---|---|---|---|---|
| | Aortic | | | |
| Test Material | Angiogenesis | Migration | Proliferation | Average |
| Isoalpha acid | Not Active | −25% | Not Active | — |
| Rho-isoalpha acid | −80% | −20% | −50% | −50% |
| Tetrahydro | −100% | −60% | −60% | −73% |
| isoalpha acid | | | | |
| Hexahydro | −95% | Not Active | −60% | — |
| isoalpha acid | | | | |
| Beta acids | −40% | −35% | −40% | −38% |
| Xanthohumol | Not Active | −60% | −60% | — |

Of the six test materials, three exhibited anti-angiogenic activity in all three assays (Table 15). THIAA was the most potent of the three followed by RIAA and BA.

The invention now having been fully described, it will be apparent to one of ordinary skill in the art that many changes and modifications can be made thereto without departing from the spirit or scope of the appended claims.

1. A method to treat a cancer responsive to protein kinase modulation in a mammal in need thereof, said method comprising administering to the mammal a therapeutically effective amount of a substituted 1,3-cyclopentadione compound.

2. The method of claim 1, wherein the substituted 1,3-cyclopentadione compound is selected from the group consisting of tetrahydro-isohumulone, tetrahydro-isocohumulone, and tetrahydro-adhumulone.

3. The method of claim 1, wherein the protein kinase modulated is selected from the group consisting of Abl(T315I), Aurora-A, Bone marrow tyrosine kinase gene in chromosome X (Bmx), Bruton's tyrosine kinase (BTK), Calcium/calmodulin-dependent protein kinase-I (CaMKI), CaMKIδ, Colon carcinoma kinase-2/cyclinA (CDK2/cyclinA), CDK3/cyclinE, CDK9/cyclin T1, Casein kinase-1(y) (CK1(y)), CK1γ1, CK1γ2, CK1γ3, CK1δ, cSRC, Death-associated protein kinase-1 (DAPK1), DAPK2, DRAK1, Ephrin receptor-A2 (EphA2), EphA8, Proto-oncogene tyrosine-protein kinase FER (Fer), Fibroblast growth factor receptor-2 (FGFR2), FGFR3, Proto-oncogene tyrosine-protein kinase FGR (Fgr), Tyrosine-protein kinase receptor FLT4 (Flt4), c-Jun NH2-terminal kinase-3 (JNK3), phosphatidylinositol-3-kinase (PI3K), Proto-oncogene serine/threonine-protein kinase-1 (Pim-1), Pim-2, Protein kinase A (PKA), PKA(b), Protein kinase B-β (PKBβ), PKBα, PKBγ, p38-regulated/activated protein kinase (PRAK), human X chromosome-encoded protein kinase X (PrKX), Ron, ribosomal S6 kinase 1 (Rsk1), ribosomal S6 kinase 2 (Rsk2), serine/threonine kinase 2 (SGK2), spleen tyrosine kinase (Syk), Tyrosine kinase with immunoglobulin and EGF repeats-2 (Tie2), TrkA, and TrkB.

4. The method of claim 1, wherein the cancer responsive to kinase modulation is selected from the group consisting of bladder, breast, cervical, colon, lung, lymphoma, melanoma, prostate, thyroid, and uterine cancer.

5. The method of claim 1, wherein the substituted 1,3-cyclopentadione compound is administered in a composition which further comprises a pharmaceutically acceptable excipient selected from the group consisting of coatings, isotonic and absorption delaying agents, binders, adhesives, lubricants, disintergrants, coloring agents, flavoring agents, sweetening agents, absorbants, detergents, and emulsifying agents.

6. The method of claim 6, wherein the composition further comprises one or more members selected from the group consisting of antioxidants, vitamins, minerals, proteins, fats, and carbohydrates.

7. The method of claim 1, wherein the substituted 1,3-cyclopentadione compound is administered in combination with a chemotherapeutic agent.

8. A method to treat angiogenic conditions responsive to protein kinase modulation in a mammal in need thereof, said method comprising administering to the mammal a therapeutically effective amount of a substituted 1,3-cyclopentadione compound.

9. The method of claim 7, wherein the substituted 1,3-cyclopentadione compound is selected from the group consisting of dihydro-(Rho) isoalpha acids; tetra-hydroisoalpha acids; hexa-hydroisoalpha acids; beta acids; their individual analogs; and mixtures thereof.

10. The method of claim 7, wherein the substituted 1,3-cyclopentadione compound is selected from the group consisting of tetrahydro-isohumulone, tetrahydro-isocohumulone, and tetrahydro-adhumulone.

11. The method of claim 7, wherein the protein kinase modulated is selected from the group consisting of ATK, Mitogen-activated protein kinase (MAPK), p38-regulated/activated protein kinase (PRAK), phosphatidylinositol-3-kinase (PI3K), Protein kinase C (PKC), Glycogen synthase kinase (GSK), Epidermal growth factor receptor (FGFR), BTK, Phosphoinositide-dependent kinase (PDK), Spleen tyrosine kinase (SYK), Mitogen- and stress-activated protein kinase (MSK) and I-kB kinase-b (IKKb).

12. The method of claim 7, wherein the substituted 1,3-cyclopentadione compound is administered in a composition which further comprises a pharmaceutically acceptable excipient selected from the group consisting of coatings, isotonic and absorption delaying agents, binders, adhesives, lubricants, disintergrants, coloring agents, flavoring agents, sweetening agents, absorbants, detergents, and emulsifying agents.

13. The method of claim 11, wherein the composition further comprises one or more members selected from the group consisting of antioxidants, vitamins, minerals, proteins, fats, and carbohydrates.

14. The method of claim 7, wherein the substituted 1,3-cyclopentadione compound is administered in combination with an anti-angiogenic agent.

15. A composition to treat a cancer responsive to protein kinase modulation in a mammal in need thereof, said composition comprising a therapeutically effective amount of a cis-n-tetrahydro-isoalpha acid (TH5) as the only substituted 1,3-cyclopentadione compound in the composition; wherein said therapeutically effective amount modulates a cancer associated protein kinase.

16. A composition to treat a cancer responsive to protein kinase modulation in a mammal in need thereof, said composition consisting essentially of therapeutically effective amounts of one or more (n) analogs of substituted 1,3-cyclopentadione compound and optionally one or more (ad) analogs of substituted 1,3-cyclopentadione compound in the composition; wherein said therapeutically effective amount modulates a cancer associated protein kinase.

17. A composition to treat a cancer responsive to protein kinase modulation in a mammal in need thereof, said composition consisting essentially of therapeutically effective amount of one or more (co) analogs of substituted 1,3-cyclopentadione compound in the composition; wherein said therapeutically effective amount modulates a cancer associated protein kinase.

18. A composition to treat angiogenic conditions responsive to protein kinase modulation in a mammal in need thereof, said composition comprising a therapeutically effective amount of a cis-n-tetrahydro-isoalpha acid (TH5) as the only substituted 1,3-cyclopentadione compound in the composition; wherein said therapeutically effective amount modulates an angiogenesis associated protein kinase.

19. A composition to treat angiogenic conditions responsive to protein kinase modulation in a mammal in need thereof, said composition consisting essentially of therapeutically effective amounts of one or more (a) analogs of substituted 1,3-cyclopentadione compound and optionally one or more (ad) analogs of substituted 1,3-cyclopentadione compound in the composition; wherein said therapeutically effective amount modulates an angiogenesis associated protein kinase.

20. A composition to treat angiogenic conditions responsive to protein kinase modulation in a mammal in need thereof, said composition consisting essentially of therapeutically effective amount of one or more (co) analogs of substituted 1,3-cyclopentadione compound in the composition; wherein said therapeutically effective amount modulates an angiogenesis associated protein kinase.

21. A composition to treat a cancer responsive to protein kinase modulation in a mammal in need thereof, said composition comprising a therapeutically effective amount of only one analog of a substituted 1,3-cyclopentadione compound; wherein said therapeutically effective amount modulates a cancer associated protein kinase.

22. A composition to treat angiogenic conditions responsive to protein kinase modulation in a mammal in need thereof, said composition comprising a therapeutically effective amount of only one analog of a substituted 1,3-cyclopentadione compound; wherein said therapeutically effective amount modulates an angiogenesis associated protein kinase.

23. The composition of claim 21 or 22, where in the analog of a substituted 1,3-cyclopentadione compound is selected from the group consisting of rho (6S) cis n iso-alpha acid, rho (6S) cis n iso-alpha acid, rho (6R) cis n iso-alpha acid, rho (6R) trans n iso-alpha acid, rho (6S) trans n iso-alpha acid, rho (6R) cis rho n iso-alpha acid, rho (6S) cis n iso-alpha acid, (6S) trans rho n iso-alpha acid, rho (6R) trans n iso-alpha acid, rho (6S) cis co iso-alpha acid, rho (6R) cis co iso-alpha acid, rho (6R) trans co iso-alpha acid, rho (6S) trans co iso-alpha acid, rho (6R) cis co iso-alpha acid, rho (6S) cis co iso-alpha acid, rho (6S) trans co iso-alpha acid, rho (6R) trans co iso-alpha acid, rho (6S) cis ad iso-alpha acid, rho (6R) cis ad iso-alpha acid, rho (6R) trans ad iso-alpha acid, rho (6S) trans ad iso-alpha acid, rho (6R) cis ad iso-alpha acid, rho (6S) cis ad iso-alpha acid, rho (6S) trans ad iso-alpha acid, rho (6R) trans ad iso-alpha acid, tetrahydro cis n iso-alpha acid, tetrahydro trans n iso-alpha acid, tetrahydro cis n iso-alpha acid, tetrahydro trans n iso-alpha acid, tetrahydro cis co iso-alpha acid, tetrahydro trans co iso-alpha acid, tetrahydro cis co iso-alpha acid, tetrahydro trans co iso-alpha acid, tetrahydro cis ad iso-alpha acid, tetrahydro trans ad iso-alpha acid, tetrahydro cis ad iso-alpha acid, tetrahydro trans ad iso-alpha acid, hexahydro (6S) cis n iso-alpha acid, hexahydro (6R) cis n iso-alpha acid, hexahydro (6R) trans n iso-alpha acid, hexahydro (6S) trans n iso-alpha acid, hexahydro (6R) cis n iso-alpha acid, hexahydro (6S) cis n iso-alpha acid, hexahydro (6S) trans n iso-alpha acid, hexahydro (6R) trans n iso-alpha acid, hexahydro (6S) cis co iso-alpha acid, hexahydro (6R) cis co iso-alpha acid, hexahydro (6R) trans co iso-alpha acid, hexahydro (6S) trans co iso-alpha acid, hexahydro (6R) cis co iso-alpha acid, hexahydro (6S) cis co iso-alpha acid, hexahydro (6S) trans co iso-alpha acid, hexahydro (6R) trans co iso-alpha acid, hexahydro (6S) cis ad iso-alpha acid, hexahydro (6R) cis ad iso-alpha acid, hexahydro (6R) trans ad iso-alpha acid, hexahydro (6S) trans ad iso-alpha acid, hexahydro (6R) cis ad iso-alpha acid, hexahydro (6S) cis ad iso-alpha acid, hexahydro (6S) trans ad iso-alpha acid, hexahydro (6R) trans ad iso-alpha acid, lupolone, colupulone, adlupulone, prelupulone, postlupulone, and xanthohumol.
